SEQUENCE LISTING (84 sequences)

SEQ ID NO: 1 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 2 - protein, 260 aa, Macaca fascicularis
MSHHWGYGKHNGPEHWHKDFPIAKGQRQSPVDIDTHTAKYDPSLKPLSVSYDQATSLRILNNGHSFNVEFDDSQDKAVIKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQMSKFRKLNFNGEGEPEELMVDNWRPAQPLKNRQIKASFK

SEQ ID NO: 3 - protein, 260 aa, Pan troglodytes
MSHHWGYGKHNGPEHWHKDFPIAKGERQSPVDIDTHTAKYDPSLKPLSVSYGQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPHGLLPESLDYWTYPGSLTTPPLLECVTWIVLKEPISVSSEQMLKFRKLNFNGEGEPEELMVDNWRPAQPLKNRQIKASFK

SEQ ID NO: 4 - protein, 260 aa, Macaca mulatta
MSHHWGYGKHNGPEHWHKDFPIAKGQRQSPVDINTHTAKYDPSLKPLSVSYDQATSLRILNNGHSFNVEFDDSQDKAVIKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQMSKFRKLNFNGEGEPEELMVDNWRPAQPLKNRQIKASFK

SEQ ID NO: 5 - protein, 260 aa, Pongo Abelii
MSHHWGYGKHNGPEHWHKDFPIAKGERQSPVDIDTHTAKYDPSLKPLSVCYDQATSLRILNNGHSFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKCADFTNFDPRGLLPASLDYWTYPGSLTTPPLLECVTWIVLKEPISVSSEQMLKFRKLNFNGEGEPEELMVDNWRPAQPLKKRQIKASFK

SEQ ID NO: 6 - protein, 260 aa, Callithrix jacchus
MSHHWGYGKHNGPEHWHKDFPIAKGERQSPVDIDTHTAKYDPSLKPLSVSYDQATSWRILNNGHSFNVEFDDSQDKAVLKGGPLDGTYRLIQFHFHWGSTDGQGSEHTVDKKKYAAELHLVHWNTKYGDFGKAAQQPDGLAVLGIFLKVGSAKPGLQKVVDVLDSIKTKGKSADFTNFDPRGLLPESLDYWTYPGSLTTPPLLESVTWIVLKEPISVSSEQILKFRKLNFSGEGEPEELMVDNWRPAQPLKNRQIKASFK

SEQ ID NO: 7 - protein, 260 aa, Lemur catta
MSHHWGYGKHNGPEHWHKDFPIAKGERQSPVDINTGAAKHDPSLKPLSVYYEQATSRRILNNGHSFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYLGSLTTPPLLECVTWIVLKEPISVSSEQMMKFRKLSFSGEGEPEELMVDNWRPAQPLKNRQIKASFK

SEQ ID NO: 8 - protein, 260 aa, Ailuropoda melanoleuca
MAHHWGYGKHNGPEHWYKDFPIAKGQRQSPVDIDTKAAIHDPALKALCPTYEQAVSQRVINNGHSFNVEFDDSQDNAVLKGGPLTGTYRLIQFHFHWGSSDGQGSEHTVDKKKYAAELHLVHWNTKYGDFGKAVQQPDGLAVLGIFLKIGDARPGLQKVLDALDSIKTKGKSADFTNFDPRGLLPESLDYWTYPGSLTTPPLLECVTWIVLKEPISVSSEQMLKFRRLNFNKEGEPEELMVDNWRPAQPLHNRQINASFK

SEQ ID NO: 9 - protein, 260 aa, Equus caballus
MSHHWGYGQHNGPKHWHKDFPIAKGQRQSPVDIDTKAAVHDAALKPLAVHYEQATSRRIVNNGHSFNVEFDDSQDKAVLQGGPLTGTYRLIQFHFHWGSSDGQGSEHTVDKKKYAAELHLVHWNTKYGDFGKAVQQPDGLAVVGVFLKVGGAKPGLQKVLDVLDSIKTKGKSADFTNFDPRGLLPESLDYWTYPGSLTTPPLLECVTWIVLREPISVSSEQLLKFRSLNFNAEGKPEDPMVDNWRPAQPLNSRQIRASFK

SEQ ID NO: 10 - protein, 260 aa, Canis lupus
MAHHWGYAKHNGPEHWHKDFPIAKGERQSPVDIDTKAAVHDPALKSLCPCYDQAVSQRIINNGHSFNVEFDDSQDKTVLKGGPLTGTYRLIQFHFHWGSSDGQGSEHTVDKKKYAAELHLVHWNTKYGEFGKAVQQPDGLAVLGIFLKIGGANPGLQKILDALDSIKTKGKSADFTNFDPRGLLPESLDYWTYPGSLTTPPLLECVTWIVLKEPISVSSEQMLKFRKLNFNKEGEPEELMMDNWRPAQPLHSRQINASFK

SEQ ID NO: 11 - protein, 260 aa, Oryctolagus cuniculus
MSHHWGYGKHNGPEHWHKDFPIANGERQSPIDIDTNAAKHDPSLKPLRVCYEHPISRRIINNGHSFNVEFDDSHDKTVLKEGPLEGTYRLIQFHFHWGSSDGQGSEHTVNKKKYAAELHLVHWNTKYGDFGKAVKHPDGLAVLGIFLKIGSATPGLQKVVDTLSSIKTKGKSVDFTDFDPRGLLPESLDYWTYPGSLTTPPLLECVTWIVLKEPITVSSEQMLKFRNLNFNKEAEPEEPMVDNWRPTQPLKGRQVKASFV

SEQ ID NO: 12 - protein, 249 aa, Ailuropoda melanoleuca
GPEHWYKDFPIAKGQRQSPVDIDTKAAIHDPALKALCPTYEQAVSQRVINNGHSFNVEFDDSQDNAVLKGGPLTGTYRLIQFHFHWGSSDGQGSEHTVDKKKYAAELHLVHWNTKYGDFGKAVQQPDGLAVLGIFLKIGDARPGLQKVLDALDSIKTKGKSADFTNFDPRGLLPESLDYWTYPGSLTTPPLLECVTWIVLKEPISVSSEQMLKFRRLNFNKEGEPEELMVDNWRPAQPLHNRQINASFK

SEQ ID NO: 13 - protein, 260 aa, Sus scrofa
MSHHWGYDKHNGPEHWHKDFPIAKGDRQSPVDINTSTAVHDPALKPLSLCYEQATSQRIVNNGHSFNVEFDSSQDKGVLEGGPLAGTYRLIQFHFHWGSSDGQGSEHTVDKKKYAAELHLVHWNTKYKDFGEAAQQPDGLAVLGVFLKIGNAQPGLQKIVDVLDSIKTKGKSVEFTGFDPRDLLPGSLDYWTYPGSLTTPPLLESVTWIVLREPISVSSGQMMKFRTLNFNKEGEPEHPMVDNWRPTQPLKNRQIRASFQ

SEQ ID NO: 14 - protein, 235 aa, Callithrix jacchus
MSHHWGYGKHNGPEHWHKDFPIAKGERQSPVDIDTHTAKYDPSLKPLSVSYDQATSWRILNNGHSFNVEFDDSQDKAVLKGGPLDGTYRLIQLHLVHWNTKYGDFGKAAQQPDGLAVLGIFLKVGSAKPGLQKVVDVLDSIKTKGKSADFTNFDPRGLLPESLDYWTYPGSLTTPPLLESVTWIVLKEPISVSSEQILKFRKLNFSGEGEPEELMVDNWRPAQPLKNRQIKASFK

SEQ ID NO: 15 - protein, 260 aa, Mus musculus
MSHHWGYSKHNGPENWHKDFPIANGDRQSPVDIDTATAQHDPALQPLLISYDKAASKSIVNNGHSFNVEFDDSQDNAVLKGGPLSDSYRLIQFHFHWGSSDGQGSEHTVNKKKYAAELHLVHWNTKYGDFGKAVQQPDGLAVLGIFLKIGPASQGLQKVLEALHSIKTKGKRAAFANFDPCSLLPGNLDYWTYPGSLTTPPLLECVTWIVLREPITVSSEQMSHFRTLNFNEEGDAEEAMVDNWRPAQPLKNRKIKASFK

SEQ ID NO: 16 - protein, 260 aa, Bos taurus
MSHHWGYGKHNGPEHWHKDFPIANGERQSPVDIDTKAVVQDPALKPLALVYGEATSRRMVNNGHSFNVEYDDSQDKAVLKDGPLTGTYRLVQFHFHWGSSDDQGSEHTVDRKKYAAELHLVHWNTKYGDFGTAAQQPDGLAVVGVFLKVGDANPALQKVLDALDSIKTKGKSTDFPNFDPGSLLPNVLDYWTYPGSLTTPPLLESVTWIVLKEPISVSSQQMLKFRTLNFNAEGEPELLMLANWRPAQPLKNRQVRGFPK

SEQ ID NO: 17 - protein, 232 aa, Oryctolagus cuniculus
GKHNGPEHWHKDFPIANGERQSPIDIDTNAAKHDPSLKPLRVCYEHPISRRIINNGHSFNVEFDDSHDKTVLKEGPLEGTYRLIQFHFHWGSSDGQGSEHTVNKKKYAAELHLVHWNTKYGDFGKAVKHPDGLAVLGIFLKIGSATPGLQKVVDTLSSIKTKGKSVDFTDFDPRGLLPESLDYWTYPGSLTTPPLLECVTWIVLKEPITVSSEQMLKFRNLNFNKEAEPEEP

SEQ ID NO: 18 - protein, 260 aa, Rattus norvegicus
MSHHWGYSKSNGPENWHKEFPIANGDRQSPVDIDTGTAQHDPSLQPLLICYDKVASKSIVNNGHSFNVEFDDSQDFAVLKEGPLSGSYRLIQFHFHWGSSDGQGSEHTVNKKKYAAELHLVHWNTKYGDFGKAVQHPDGLAVLGIFLKIGPASQGLQKITEALHSIKTKGKRAAFANFDPCSLLPGNLDYWTYPGSLTTPPLLECVTWIVLKEPITVSSEQMSHFRKLNFNSEGEAEELMVDNWRPAQPLKNRKIKASFK

SEQ ID NO: 19 - protein, 208 aa, Homo sapiens
MSLSITNNGHSVQVDFNDSDDRTVVTGGPLEGPYRLKQFHFHWGKKHDVGSEHTVDGKSFPSELHLVHWNAKKYSTFGEAASAPDGLAVVGVFLETGDEHPSMNRLTDALYMVRFKGTKAQFSCFNPKCLLPASRHYWTYPGSLTTPPLSESVTWIVLREPICISERQMGKFRSLLFTSEDDERIHMVNNFRPPQPLKGRVVKASFRA

SEQ ID NO: 20 - protein, 264 aa, Pongo Abelii
MTGHHGWGYGQDDGPSHWHKLYPIAQGDRQSPINIISSQAVYSPSLQPLELSYEACMSLSITNNGHSVQVDFNDSDDRTVVTGGPLEGPYRLKQFHFHWGKKHDVGSEHTVDGKSFPSELHLVHWNAKKYSTFGEAASAPDGLAVVGVFLETGDEHPSMNRLTDALYMVRFKGTKAQFSCFNPKSLLPASRHYWTYPGSLTTPPLSESVTWIVLREPICISERQMGKFRSLLFTSEDDERIHMVNNFRPPQPLKGRVVKASFRA

SEQ ID NO: 21 - protein, 312 aa, Pan troglodytes
MEFGLSPELSPSRCFKRLLRGSERGRSRSPNERTEPTGQVHGCGDGSGMTGHHGWGYGQDDGPSHWHKLYPIAQGDRQSPINIISSQAVYSPSLQPLELSYEACMSLSITNNGHSVQVDFNDSDDRTVVTGGPLEGPYRLKQFHFHWGKKHDVGSEHTVDGKSFPSELHLVHWNAKKYSTFGEAASAPDGLAVVGVFLETGDEHPSMNRLTDALYMVRFKGTKAQFSCFNPKCLLPASRHYWTYPGSLTTPPLSESVTWIVLREPICISERQMRKFRSLLFTSEDDERIHMVNNFRPPQPLKGRVVKASFRA

SEQ ID NO: 22 - protein, 264 aa, Callithrix jacchus
MTGHHGWGYGQDDGPSHWHKLYPIAQGDRQSPINIISSQAVYSPSLQPLELSYEACMSLSITNNGHSVQVDFNDSDDRTVVTGGPLEGPYRLKQFHFHWGKKHDVGSEHTVDGKSFPSELHLVHWNAKKYSTFGEAASAPDGLAVVGVFLETGDEHPSMNRLTDALYMVRFKGTKAQFSCFNPKCLLPASWHYWTYPGSLTTPPLSESVTWIVLREPICISERQMGKFRSLLFTSEDDERVHMVNNFRPPQPLKGRVVKASFRA

SEQ ID NO: 23 - protein, 251 aa, Ailuropoda melanoleuca
GPSQWHKLYPIAQGDRQSPINIVSSQAVYSPSLKPLELSYEACISLSIANNGHSVQVDFNDSDDRTVVTGGPLDGPYRLKQFHFHWGKKHSVGSEHTVDGKSFPSELHLVHWNAKKYSTFGEAASAPDGLAVVGVFLETGDEHPSMNRLTDALYMVRFKGTKAQFSCFNPKCLLPASRHYWTYPGSLTTPPLSESVTWIVLREPISISERQMEKFRSLLFTSEDDERIHMVNNFRPPQPLKGRVVKASFRA

SEQ ID NO: 24 - protein, 278 aa, Canis familiaris
MTGHHCWGYGQNDEIQASLSPSLSTPAGPSQWHKLYPIAQGDRQSPINIVSSQAVYSPSLKPLELSYEACISLSITNNGHSVQVDFNDSDDRTAVTGGPLDGPYRLKQLHFHWGKKHSVGSEHTVDGKSFPSELHLVHWNAKKYSTFGEAASAPDGLAVVGIFLETGDEHPSMNRLTDALYMVRFKGTKAQFSCFNPKCLLPASRHYWTYPGSLTTPPLSESVTWIVLREPISISERQMEKFRSLLFTSEEDERIHMVNNFRPPQPLKGRVVKASFRA

SEQ ID NO: 25 - protein, 264 aa, Bos taurus
MTGHHGWGYGQNDGPSHWHKLYPIAQGDRQSPINIVSSQAVYSPSLKPLEISYESCTSLSIANNGHSVQVDFNDSDDRTVVSGGPLDGPYRLKQFHFHWGKKHGVGSEHTVDGKSFPSELHLVHWNAKKYSTFGEAASAPDGLAVVGVFLETGDEHPSMNRLTDALYMVRFKGTKAQFSCFNPKCLLPASRHYWTYPGSLTTPPLSESVTWIVLREPIRISERQMEKFRSLLFTSEEDERIHMVNNFRPPQPLKGRVVKASFRA

SEQ ID NO: 26 - protein, 271 aa, Rattus norvegicus
MTVLWWPMLREELMSKLRTGGPSNWHKLYPIAQGDRQSPINIISSQAVYSPSLQPLELFYEACMSLSITNNGHSVQVDFNDSDDRTVVAGGPLEGPYRLKQLHFHWGKKRDVGSEHTVDGKSFPSELHLVHWNAKKYSTFGEAAAAPDGLAVVGIFLETGDEHPSMNRLTDALYMVRFKDTKAQFSCFNPKCLLPTSRHYWTYPGSLTTPPLSESVTWIVLREPIRISERQMEKFRSLLFTSEDDERIHMVNNFRPPQPLKGRVVKASFQS

SEQ ID NO: 27 - protein, 266 aa, Oryctolagus cuniculus
MTGHHGWGYGQDDGGRPSHWHKLYPIAQGDRQSPINIVSSQAVYSPGLQPLELSYEACTSLSIANNGHSVQVDFNDSDDRTVVTGGPLEGPYRLKQFHFHWGKRRDAGSEHTVDGKSFPSELHLVHWNARKYSTFGEAASAPDGLAVVGVFLETGNEHPSMNRLTDALYMVRFKGTKAQFSCFNPKCLLPSSRHYWTYPGSLTTPPLSESVTWIVLREPISISERQMEKFRSLLFTSEDDERVHMVNNFRPPQPLRGRVVKASFRA

SEQ ID NO: 28 - protein, 255 aa, Mus musculus
GQDDGPSNWHKLYPIAQGDRQSPINIISSQAVYSPSLQPLELFYEACMSLSITNNGHSVQVDFNDSDDRTVVSGGPLEGPYRLKQLHFHWGKKRDMGSEHTVDGKSFPSELHLVHWNAKKYSTFGEAAAAPDGLAVVGVFLETGDEHPSMNRLTDALYMVRFKDTKAQFSCFNPKCLLPTSRHYWTYPGSLTTPPLSESVTWIVLREPIRISERQMEKFRSLLFTSEDDERIHMVDNFRPPQPLKGRVVKASFQA

SEQ ID NO: 29 - protein, 264 aa, Monodelphis domestica
MTGHHGWGYGQEDGPSEWHKLYPIAQGDRQSPIDIVSSQAVYDPTLKPLVLAYESCMSLSIANNGHSVMVEFDDVDDRTVVNGGPLDGPYRLKQFHFHWGKKHSLGSEHTVDGKSFSSELHLVHWNGKKYKTFAEAAAAPDGLAVVGIFLETGDEHASMNRLTDALYMVRFKGTKAQFNSFNPKCLLPMNLSYWTYPGSLTTPPLSESVTWIVLKEPITISEKQMEKFRSLLFTAEEDEKVRMVNNFRPPQPLKGRVVQASFRS

SEQ ID NO: 30 - protein, 264 aa, Gallus gallus
MTGHHSWGYGQDDGPAEWHKSYPIAQGNRQSPIDIISAKAVYDPKLMPLVISYESCTSLNISNNGHSVMVEFEDIDDKTVISGGPFESPFRLKQFHFHWGAKHSEGSEHTIDGKPFPCELHLVHWNAKKYATFGEAAAAPDGLAVVGVFLEIGKEHANMNRLTDALYMVKFKGTKAQFRSFNPKCLLPLSLDYWTYLGSLTTPPLNESVIWVVLKEPISISEKQLEKFRMLLFTSEEDQKVQMVNNFRPPQPLKGRTVRASFKA

SEQ ID NO: 31 - protein, 264 aa, Taeniopygia guttata
MTGQHSWGYGQADGPSEWHKAYPIAQGNRQSPIDIDSARAVYDPSLQPLLISYESCSSLSISNTGHSVMVEFEDTDDRTAISGGPFQNPFRLKQFHFHWGTTHSQGSEHTIDGKPFPCELHLVHWNARKYTTFGEAAAAPDGLAVVGVFLEIGKEHASMNRLTDALYMVKFKGTKAQFRGFNPKCLLPLSLDYWTYLGSLTTPPLNESVTWIVLKEPIRISVKQLEKFRMLLFTGEEDQRIQMANNFRPPQPLKGRIVRASFKA

SEQ ID NO: 32 - protein, 262 aa, Homo sapiens
MSRLSWGYREHNGPIHWKEFFPIADGDQQSPIEIKTKEVKYDSSLRPLSIKYDPSSAKIISNSGHSFNVDFDDTENKSVLRGGPLTGSYRLRQVHLHWGSADDHGSEHIVDGVSYAAELHVVHWNSDKYPSFVEAAHEPDGLAVLGVFLQIGEPNSQLQKITDTLDSIKEKGKQTRFTNFDLLSLLPPSWDYWTYPGSLTVPPLLESVTWIVLKQPINISSQQLAKFRSLLCTAEGEAAAFLVSNHRPPQPLKGRKVRASFH

SEQ ID NO: 33 - protein, 262 aa, Pan troglodytes
MSRLSWGYREHNGPIHWKEFFPIADGDQQSPIEIKTKEVKYDSSLRPLSIKYDPSSAKIISNSGHSFNVDFDDTENKSVLRGGPLTGSYRLRQFHLHWGSADDHGSEHIVDGVSYAAELHVVHWNSDKYPSFVEAAHEPDGLAVLGVFLQIGEPNSQLQKITDTLDSIKEKGKQTRFTNFDPLSLLPPSWDYWTYPGSLTVPPLLESVTWIVLKQPINISSQQLAKFRSLLCTAEGEAAAFLVSNHRPPQPLKGRKVRASFH

SEQ ID NO: 34 - protein, 262 aa, Macaca mulatta
MSRLSWGYREHNGPIHWKEFFPIADGDQQSPIEIKTQEVKYDSSLRPLSIKYDPSSAKIISNSGHSFNVDFDDTEDKSVLRGGPLAGSYRLRQFHLHWGSADDHGSEHIVDGVSYAAELHVVHWNSDKYPSFVEAAHEPDGLAVLGVFLQIGEPNSQLQKITDILDSIKEKGKQTRFTNFDPLSLLPPSWDYWTYPGSLTVPPLLESVIWIVLKQPINVSSQQLAKFRSLLCTAEGEAAAFLLSNHRPPQPLKGRKVRASFR

SEQ ID NO: 35 - protein, 262 aa, Oryctolagus cuniculus
MSRISWGYGEHNGPIHWNQFFPIADGDQQSPIEIKTKEVKYDSSLRPLSIKYDPSSAKIISNSGHSFNVDFDDTEDKSVLRGGPLTGNYRLRQFHLHWGSADDHGSEHVVDGVRYAAELHVVHWNSDKYPSFVEAAHEPDGLAVLGVFLQIGEYNSQLQKITDILDSIKEKGKQTRFTNFDPLSLLPSSWDYWTYPGSLTVPPLLESVTWIVLKQPINISSQQLAKFRSLLCSAEGESAAFLLSNHRPPQPLKGRKVRASFH

SEQ ID NO: 36 - protein, 262 aa, Ailuropoda melanoleuca
MSRLSWGYGEHNGPIHWNKFFPIADGDQQSPIEIKTKEVKYDSSLRPLSIKYDANSAKIISNSGHSFSVDFDDTEDKSVLRGGPLTGSYRLRQFHLHWGSADDHGSEHVVDGVRYAAELHVVHWNSDKYPSFVEAAHEPDGLAVLGVFLQIGEHNSQLQKITDILDSIKEKGKQTRFTNFDPLSLLPPSWDYWTYPGSLTVPPLLESVTWIVLKQPINISSEQLATFRTLLCTAEGEAAAFLLSNHRPPQPLKGRKVRASFH

SEQ ID NO: 37 - protein, 262 aa, Sus scrofa
MSRFSWGYGEHNGPVHWNEFFPIADGDQQSPIEIKTKEVKYDSSLRPLSIKYDPSSAKIISNSGHSFSVDFDDTEDKSVLRGGPLTGSYRLRQFHLHWGSADDHGSEHVVDGVKYAAELHVVHWNSDKYPSFVEAAHEPDGLAVLGVFLQIGEHNSQLQKITDILDSIKEKGKQTRFTNFDPLSLLPPSWDYWTYPGSLTVPPLLESVTWIILKQPINISSQQLATFRTLLCTKEGEEAAFLLSNHRPLQPLKGRKVRASFH

SEQ ID NO: 38 - protein, 262 aa, Callithrix jacchus
MSRLSWGYGEHNGPIHWNEFFPIADGDRQSPIEIKAKEVKYDSSLRPLSIKYDPSSAKIISNSGHSFNVDFDDTEDKSVLHGGPLTGSYRLRQFHLHWGSADDHGSEHVVDGVRYAAELHVVHWNSEKYPSFVEAAHEPDGLAVLGVFLQIGEPNSQLQKIIDILDSIKEKGKQIRFTNFDPLSLFPPSWDYWTYSGSLTVPPLLESVTWILLKQPINISSQQLAKFRSLLCTAEGEAAAFLLSNYRPPQPLKGRKVRASFR

SEQ ID NO: 39 - protein, 262 aa, Rattus norvegicus
MARLSWGYDEHNGPIHWNELFPIADGDQQSPIEIKTKEVKYDSSLRPLSIKYDPASAKIISNSGHSFNVDFDDTEDKSVLRGGPLTGSYRLRQFHLHWGSADDHGSEHVVDGVRYAAELHVVHWNSDKYPSFVEAAHESDGLAVLGVFLQIGEHNPQLQKITDILDSIKEKGKQTRFTNFDPLCLLPSSWDYWTYPGSLTVPPLLESVTWIVLKQPISISSQQLARFRSLLCTAEGESAAFLLSNHRPPQPLKGRRVRASFY

SEQ ID NO: 40 - protein, 262 aa, Mus musculus
MARLSWGYGEHNGPIHWNELFPIADGDQQSPIEIKTKEVKYDSSLRPLSIKYDPASAKIISNSGHSFNVDFDDTEDKSVLRGGPLTGNYRLRQFHLHWGSADDHGSEHVVDGVRYAAELHVVHWNSDKYPSFVEAAHESDGLAVLGVFLQIGEHNPQLQKITDILDSIKEKGKQTRFTNFDPLCLLPSSWDYWTYPGSLTVPPLLESVTWIVLKQPISISSQQLARFRSLLCTAEGESAAFLLSNHRPPQPLKGRRVRASFY

SEQ ID NO: 41 - protein, 279 aa, Canis familiaris
MPPRRHGPNTFLSAGTKGQQNFWTKNQKSGPIHWNKFFPIADGDQQSPIEIKTKEVKYDSSLRPLSIKYDANSAKIISNSGHSFSVDFDDTEDKSVLRGGPLTGSYRLRQFHLHWGSADDHGSEHVVDGVRYAAELHVVHWNSDKYPSFVEAAHEPDGLAVLGVFLQIGEHNSQLQKITDILDSIKEKGKQTRFTNFDPLSLLPPSWDYWTYPGSLTVPPLLESVTWIVLKQPINISSQQLATFRTLLCTAEGEAAAFLLSNHRPPQPLKGRKVRASFH

SEQ ID NO: 42 - protein, 252 aa, Equus caballus
MSGPVHWNEFFPIADGDQQSPIEIKTKEVKYDSSLRPLTIKYDPSSAKIISNSGHSFSVGFDDTENKSVLRGGPLTGSYRLRQFHLHWGSADDHGSEHVVDGVRYAAELHIVHWNSDKYPSFVEAAHEPDGLAVLGVFLQVGEHNSQLQKITDTLDSIKEKGKQTLFTNFDPLSLLPPSWDYWTYPGSLTVPPLLESVTWIILKQPINISSQQLVKFRTLLCTAEGETAAFLLSNHRPPQPLKGRKVRASFR

SEQ ID NO: 43 - protein, 262 aa, Bos taurus
MSGFSWGYGERDGPVHWNEFFPIADGDQQSPIEIKTKEVRYDSSLRPLGIKYDASSAKIISNSGHSFNVDFDDTDDKSVLRGGPLTGSYRLRQFHLHWGSTDDHGSEHVVDGVRYAAELHVVHWNSDKYPSFVEAAHEPDGLAVLGIFLQIGEHNPQLQKITDILDSIKEKGKQTRFTNFDPVCLLPPCRDYWTYPGSLTVPPLLESVTWIILKQPINISSQQLAAFRTLLCSREGETAAFLLSNHRPPQPLKGRKVRASFR

SEQ ID NO: 44 - protein, 262 aa, Monodelphis domestica
MSRLSWGYCEHNGPVHWSELFPIADGDYQSPIEINTKEVKYDSSLRPLSIKYDPASAKIISNSGHSFSVDFDDSEDKSVLRGGPLIGTYRLRQFHLHWGSTDDQGSEHTVDGMKYAAELHVVHWNSDKYPSFVEAAHEPDGLAVLGIFLQTGEHNLQMQKITDILDSIKEKGKQIRFTNFDPATLLPQSWDYWTYPGSLTVPPLLESVTWIVLKQPITISSQQLAKFRSLLYTGEGEAAAFLLSNYRPPQPLKGRKVRASFR

SEQ ID NO: 45 - protein, 483 aa, Ornithorhynchus anatinus
MKKGVGSFYELAVNRWSVVNRVQIMIVESITEPLLCGSRALALTLSPTQALAVAPALALAVVQALALTVVQALALAVSPALALSVAPALALAVVQALALAVVQALALAVAQALALAVAQALALAVAQALALALPQALALTLPQALALTLSPTLALSVAPALALAVAPALALADSPALALALARPHPSSGSSPALDCELVLFGDCHTVLLKWMRMGNYSSVSPLEERNSSCPLGPIHWNELFPIADGDRQSPIEIKTKEVKYDSSLRPLSIKYDPTSAKIISNSGHSFSVDFDDTEDKSVLRGGPLSGTYRLRQFHFHWGSADDHGSEHTVDGMEYSAELHVVHWNSDKYSSFVEAAHEPDGLAVLGIFLKRGEHNLQLQKITDILDAIKEKGKQMRFTNFDPLSLLPLTRDYWTYPGSLTVPPLLESVIWIIFKQPISISSQQLAKFRNLLYTAEGEAADFMLSNHRPPQPLKGRKVRASFRS

SEQ ID NO: 46 - DNA, 783 bp, Homo sapiens
atgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccttggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaataa

SEQ ID NO: 47 - DNA, 795 bp, Artificial Sequence
  Synthesized
gaattcatgtctcatcattggggttatggtaaacacaatggtcctgaacactggcataaagactttccaattgcaaaaggtgaacgtcaatcacctgttgatattgacactcatacagctaaatatgacccttctttaaaaccattatctgtttcatatgatcaagcaacttctttacgtattttaaacaatggtcatgcttttaatgtagaatttgatgactctcaagataaagcagtattaaaaggtggtccattagatggtacttaccgtttaattcaatttcactttcactggggttcattagatggtcaaggttcagaacatactgtagataaaaaaaaatatgctgcagaattacacttagttcactggaacacaaaatatggtgattttggtaaagctgtacaacaacctgatggtttagctgttttaggtatttttttaaaagttggtagtgctaaaccaggtcttcaaaaagttgttgatgtattagattcaattaaaacaaaaggtaaaagtgctgactttactaatttcgatcctcgtggtttacttcctgaatctttagattactggacatatccaggttcattaacaacacctcctcttttagaatgtgtaacatggattgtattaaaagaaccaattagtgtaagtagtgaacaagtattaaaattccgtaaacttaatttcaatggtgaaggtgaaccagaagaattaatggttgataactggcgtccagctcaaccattaaaaaatcgtcaaattaaagcttcattcaaataagcatgc

SEQ ID NO: 48 - protein, 475 aa, Chlamydomonas reinhardtii
MVPQTETKAGAGFKAGVKDYRLTYYTPDYVVRDTDILAAFRMTPQLGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 49 - protein, 479 aa, Arabidopsis thaliana
MSPQTETKASVGFKAGVKEYKLTYYTPEYETKDTDILAAFRVTPQPGVPPEEAGAAVAAESSTGTWTTVWTDGLTSLDRYKGRCYHIEPVPGEETQFIAYVAYPLDLFEEGSVTNMFTSIVGNVFGFKALAALRLEDLRIPPAYTKTFQGPPHGIQVERDKLNKYGRPLLGCTIKPKLGLSAKNYGRAVYECLRGGLDFTKDDENVNSQPFMRWRDRFLFCAEAIYKSQAETGEIKGHYLNATAGTCEEMIKRAVFARELGVPIVMHDYLTGGFTANTSLSHYCRDNGLLLHIHRAMHAVIDRQKNHGMHFRVLAKALRLSGGDHIHAGTVVGKLEGDRESTLGFVDLLRDDYVEKDRSRGIFFTQDWVSLPGVLPVASGGIHVWHMPALTEIFGDDSVLQFGGGTLGHPWGNAPGAVANRVALEACVQARNEGRDLAVEGNEIIREACKWSPELAAACEVWKEITFNFPTIDKLDGQE

SEQ ID NO: 50 - protein, 479 aa, Capsella bursa-pastoris
MSPQTETKASVGFKAGVKEYKLTYYTPEYETKDTDILAAFRVTPQPGVPPEEAGAAVAAESSTGTWTTVWTDGLTSLDRYKGRCYHIEPVPGEETQFIAYVAYPLDLFEEGSVTNMFTSIVGNVFGFKALAALRLEDLRIPPAYTKTFQGPPHGIQVERDKLNKYGRPLLGCTIKPKLGLSAKNYGRAVYECLRGGLDFTKDDENVNSQPFMRWRDRFLFCAEAIYKSQAETGEIKGHYLNATAGTCEEMIKRAVFARELGVPIVMHDYLTGGFTANTSLSHYCRDNGLLLHIHRAMHAVIDRQKNHGMHFRVLAKALRLSGGDHIHAGTVVGKLEGDRESTLGFVDLLRDDYVEKDRSRGIFFTQDWVSLPGVLPVASGGIHVWHMPALTEIFGDDSVLQFGGGTLGHPWGNAPGAVANRVALEACVQARNEGRDLAVEGNEIIREACKWSPELAAACEVWKEIRFNFPTIDKLDGQE

SEQ ID NO: 51 - protein, 479 aa, Crucihimalaya wallichii
MSPQTETKASVGFKAGVKEYKLTYYTPEYETKDTDILAAFRVTPQPGVPPEEAGAAVAAESSTGTWTTVWTDGLTSLDRYKGRCYHIEPVPGEETQFIAYVAYPLDLFEEGSVTNMFTSIVGNVFGFKALAALRLEDLRIPPAYTKTFQGPPHGIQVERDKLNKYGRPLLGCTIKPKLGLSAKNYGRAVYECLRGGLDFTKDDENVNSQPFMRWRDRFLFCAEAIYKSQAETGEIKGHYLNATAGTCEEMIKRAVFARELGVPIVMHDYLTGGFTANTSLAHYCRDNGLLLHIHRAMHAVIDRQKNHGMHFRVLAKALRLSGGDHIHAGTVVGKLEGDRESTLGFVDLLRDDYVEKDRSRGIFFTQDWVSLPGVLPVASGGIHVWHMPALTEIFGDDSVLQFGGGTLGHPWGNAPGAVANRVALEACVQARNEGRDLAVEGNEIIREACKWSPELAAACEVWKEIRFNFPTIDKLDGQE

SEQ ID NO: 52 - protein, 479 aa, Arabis hirsuta
MSPQTETKASVGFKAGVKEYKLTYYTPEYETKDTDILAAFRVTPQPGVPPEEAGAAVAAESSTGTWTTVWTDGLTSLDRYKGRCYHIEPVPGEETQFIAYVAYPLDLFEEGSVTNMFTSIVGNVFGFKALAALRLEDLRIPPAYTKTFQGPPHGIQVERDKLNKYGRPLLGCTIKPKLGLSAKNYGRAVYECLRGGLDFTKDDENVNSQPFMRWRDRFLFCAEAIYKSQAETGEIKGHYLNATAGTCEEMIKRAVFARELGVPIVMHDYLTGGFTANTSLAHYCRDNGLLLHIHRAMHAVIDRQKNHGMHFRVLAKALRLSGGDHVHAGTVVGKLEGDRESTLGFVDLLRDDYVEKDRSRGIFFTQDWVSLPGVLPVASGGIHVWHMPALTEIFGDDSVLQFGGGTLGHPWGNAPGAVANRVALEACVQARNEGRDLAVEGNEIIREACKWSPELAAACEVWKEIRFNFPTVDKLDGQE

SEQ ID NO: 53 - protein, 479 aa, Draba nemorosa
MSPQTETKASVGFKAGVKEYKLTYYTPEYETKDTDILAAFRVTPQPGVPPEEAGAAVAAESSTGTWTTVWTDGLTSLDRYKGRCYHIEPVPGEETQFIAYVAYPLDLFEEGSVTNMFTSIVGNVFGFKALAALRLEDLRIPPAYTKTFQGPPHGIQVERDKLNKYGRPLLGCTIKPKLGLSAKNYGRAVYECLRGGLDFTKDDENVNSQPFMRWRDRFLFCAEAIYKSQAETGEIKGHYLNATAGTCEEMIKRAVFARELGVPIVMHDYLTGGFTANTSLSHYCRDNGLLLHIHRAMHAVIDRQKNHGMHFRVLAKALRLSGGDHIHAGTVVGKLEGDRESTLGFVDLLRDDYVEKDRSRGIFFTQDWVSLPGVLPVASGGIHVWHMPALTEIFGDDSVLQFGGGTLGHPWGNAPGAVANRVALEACVQARNEGRDLAVEGNEIIREACKWSPELAAACEVWKEIRFNFPTIDKLDGQA

SEQ ID NO: 54 - protein, 479 aa, Lobularia maritima
MSPQTETKASVGFKAGVKEYKLTYYTPEYETKDTDILAAFRVTPQPGVPPEEAGAAVAAESSTGTWTTVWTDGLTSLDRYKGRCYHIEPVPGEETQFIAYVAYPLDLFEEGSVTNMFTSIVGNVFGFKALAALRLEDLRIPPAYTKTFQGPPHGIQVERDKLNKYGRPLLGCTIKPKLGLSAKNYGRAVYECLRGGLDFTKDDENVNSQPFMRWRDRFLFCAEAIYKSQAETGEIKGHYLNATAGTCEEMIKRAVFARELGVPIVMHDYLTGGFTANTSLAHYCRDNGLLLHIHRAMHAVIDRQKNHGMHFRVLAKALRLSGGDHIHAGTVVGKLEGDRESTLGFVDLLRDDYIEKDRSRGIFFTQDWVSLPGVLPVASGGIHVWHMPALTEIFGDDSVLQFGGGTLGHPWGNAPGAVANRVALEACVQARNEGRDLAVEGNEIVREACKWSPELAAACEVWKEIRFNFPTIDKLDGQE

SEQ ID NO: 55 - protein, 411 aa, Chlamydomonas reinhardtii
MAQALALADRFKGLKELPGLKADACGVQRMTGDVGERVAIVAARDVRDKETVMVIPENLAVTRVDAESHPVVGPLAAEASELTALTLWLLAERAAGAGSNYAGLLATLPESTLSPLLWSDAELEELMAGSPVLPEARSRKKALADTWAALAPKLAADPARFPAGRRAAGARKGVVVWDGAGSEMLLNDGRPNGELLLATGTLQDNNSSDFLSWPAGLVPADRYYMMKSQVLESMGYSAAEEFPVYADRMPIQLLAYLRLSRVADPALLAKCTFEADVELSQMNEYEILQILMGDCRERLASYTKSYEEDVKIAQQSDLSPKERLAVKLRLGEKRIINATMEAVRRRLAPIRGIPTKSGQLADPNSDLKEIFDTIESIPTAPLRLMQGLVSWARGDDDPEWYGKKKPGQGRK

SEQ ID NO: 56 - protein, 181 aa, Arabidopsis thaliana
MASSMLSSAAVVTSPAQATMVAPFTGLKSSASFPVTRKANNDITSITSNGGRVSCMKVWPPIGKKKFETLSYLPDLTDVELAKEVDYLLRNKWIPCVEFELEHGFVYREHGNTPGYYDGRYWTMWKLPLFGCTDSAQVLKEVEECKKEYPGAFIRIIGFDNTRQVQCISFIAYKPPSFTDA

SEQ ID NO: 57 - protein, 181 aa, Brassica napus
MASSMLSSAAVVTSPAQATMVAPFTGLKSSAAFPVTRKANNDITSIASNGGRVSCMKVWPPVGKKKFETLSYLPDLTEVELGKEVDYLLRNKWIPCVEFELEHGFVYREHGSTPGYYDGRYWTMWKLPLFGCTDSAQVLKEVQECKTEYPNAFIRIIGFDNNRQVQCISFIAYKPPSFTGA

SEQ ID NO: 58 - protein, 181 aa, Raphanus sativus
MASSMLSSAAVVTSQLQATMVAPFTGLKSSAAFPVTRKTNTDITSIASNGGRVSCMKVWPPIGKKKFETLSYLPDLSDVELAKEVDYLLRNKWIPCVEFELEHGFVYREHGSTPGYYDGRYWTMWKLPLFGCTDSAQVLKEVQECKKEYPNALIRIIGFDNNRQVQCISFIAYKPPSFTDA

SEQ ID NO: 59 - protein, 181 aa, Arabidopsis thaliana
MASSMFSSTAVVTSPAQATMVAPFTGLKSSASFPVTRKANNDITSITSNGGRVSCMKVWPPIGKKKFETLSYLPDLSDVELAKEVDYLLRNKWIPCVEFELEHGFVYREHGNTPGYYDGRYWTMWKLPLFGCTDSAQVLKEVEECKKEYPGAFIRIIGFDNTRQVQCISFIAYKPPSFTEA

SEQ ID NO: 60 - protein, 181 aa, Arabidopsis thaliana
MASSMLSSAAVVTSPAQATMVAPFTGLKSSAAFPVTRKTNKDITSIASNGGRVSCMKVWPPIGKKKFETLSYLPDLSDVELAKEVDYLLRNKWIPCVEFELEHGFVYREHGNTPGYYDGRYWTMWKLPLFGCTDSAQVLKEVEECKKEYPGAFIRIIGFDNTRQVQCISFIAYKPPSFTEA

SEQ ID NO: 61 - protein, 181 aa, Brassica napus
MAYSMLSSAAVVTSPAQATMVAPFTGLKSSAAFPVTRKANNDITSIASNGGRVSCMKVWPPVGKKKFETLSYLPDLTEVELGKEVDYLLRNKWIPCVEFELEHGFVYREHGSTPGYYDGRYWTMWKLPLFGCTDSAQVLKEVQECKTEYPNAFIRIIGFDNNRQVQCISFIAYKPPSFTGA

SEQ ID NO: 62 - protein, 181 aa, Brassica rapa
MAYSMLSSAAVVTSPAQATMVAPFTGLKSSSAFPVTRKANNDITSIVSNGGRVSCMKVWPPVGKKKFETLSYLPDLTEVELGKEVDYLLRNKWIPCVEFELEHGFVYREHGSTPGYYDGRYWTMWKLPLFGCTDSAQVLKEVQECKTEYPNAFIRIIGFDNNRQVQCISFIAYKPPSFTGA

SEQ ID NO: 63 - protein, 181 aa, Ricinus communis
MASSMISSASVSRSSPAQATMVAPFTGLKSAASFPVTRKANNDITSIASNGGRVQCMQVWPPLGKKKFETLSYLPDLTDEQLAKEVDYLLRKGWIPCLEFELEHGFVYRENHRSPGYYDGRYWTMWKLPMFGCSDSTQVLKELDEAKKAYPNSFIRIIGFDNRRQVQCISFIAYKPTTFNS

SEQ ID NO: 64 - protein, 5 aa, Artificial Sequence
  Synthesized
FPXXP

SEQ ID NO: 65 - protein, 4 aa, Artificial Sequence
  Synthesized
PPXX

SEQ ID NO: 66 - protein, 5 aa, Artificial Sequence
  Synthesized
PPPPY

SEQ ID NO: 67 - protein, 4 aa, Artificial Sequence
  Synthesized
PPLP

SEQ ID NO: 68 - protein, 7 aa, Artificial Sequence
  Synthesized
RPLPVAP

SEQ ID NO: 69 - protein, 10 aa, Artificial Sequence
  Synthesized
PPPALPPKKR

SEQ ID NO: 70 - protein, 8 aa, Artificial Sequence
  Synthesized
RKGDYASY

SEQ ID NO: 71 - protein, 5 aa, Artificial Sequence
  Synthesized
WXXQF

SEQ ID NO: 72 - protein, 7 aa, Artificial Sequence
  Synthesized
PPPPGHR

SEQ ID NO: 73 - protein, 723 aa, Homo sapiens
MGLADASGPRDTQALLSATQAMDLRRRDYHMERPLLNQEHLEELGRWGSAPRTHQWRTWLQCSRARAYALLLQHLPVLVWLPRYPVRDWLLGDLLSGLSVAIMQLPQGLAYALLAGLPPVFGLYSSFYPVFIYFLFGTSRHISVATPGPLPLLTAPGRPTGGAGPDPLRLRGHLPVRTSCPRLYHSCSCAGLRLTAQVCVWPPSEQPLWATVPHLLLEVCWKLPQSKVGTVVTAAVAGVVLVVVKLLNDKLQQQLPMPIPGELLTLIGATGISYGMGLKHRFEVDVVGNIPAGLVPPVAPNTQLFSKLVGSAFTIAVVGFAIAISLGKIFALRHGYRVDSNQELVALGLSNLIGGIFQCFPVSCSMSRSLVQESTGGNSQVAGAISSLFILLIIVKLGELFHDLPKAVLAAIIIVNLKGMLRQLSDMRSLWKANRADLLIWLVTFTATILLNLDLGLVVAVIFSLLLVVVRTQMPHYSVLGQVPDTDIYRDVAEYSEAKEVRGVKVFRSSATVYFANAEFYSDALKQRCGVDVDFLISQKKKLLKKQEQLKLKQLQKEEKLRKQAASPKGASVSINVNTSLEDMRSNNVEDCKMMQVSSGDKMEDATANGQEDSKAPDGSTLKALGLPQPDFHSLILDLGALSFVDTVCLKSLKNIFHDFREIEVEVYMAACHSPVVSQLEAGHFFDASITKKHLFASVHDAVTFALQHPRPVPDSPVSVTRL

SEQ ID NO: 74 - protein, 759 aa, Homo sapiens
MGLADASGPRDTQALLSATQAMDLRRRDYHMERPLLNQEHLEELGRWGSAPRTHQWRTWLQCSRARAYALLLQHLPVLVWLPRYPVRDWLLGDLLSGLSVAIMQLPQGLAYALLAGLPPVFGLYSSFYPVFIYFLFGTSRHISVGTFAVMSVMVGSVTESLAPQALNDSMINETARDAARVQVASTLSVLVGLFQVGLGLIHFGFVVTYLSEPLVRGYTTAAAVQVFVSQLKYVFGLHLSSHSGPLSLIYTVLEVCWKLPQSKVGTVVTAAVAGVVLVVVKLLNDKLQQQLPMPIPGELLTLIGATGISYGMGLKHRFEVDVVGNIPAGLVPPVAPNTQLFSKLVGSAFTIAVVGFAIAISLGKIFALRHGYRVDSNQELVALGLSNLIGGIFQCFPVSCSMSRSLVQESTGGNSQVAGAISSLFILLIIVKLGELFHDLPKAVLAAIIIVNLKGMLRQLSDMRSLWKANRADLLIWLVTFTATILLNLDLGLVVAVIFSLLLVVVRTQMPHYSVLGQVPDTDIYRDVAEYSEAKEVRGVKVFRSSATVYFANAEFYSDALKQRCGVDVDFLISQKKKLLKKQEQLKLKQLQKEEKLRKQAASPKGASVSINVNTSLEDMRSNNVEDCKMMQVSSGDKMEDATANGQEDSKAPDGSTLKALGLPQPDFHSLILDLGALSFVDTVCLKSLKNIFHDFREIEVEVYMAACHSPVVSQLEAGHFFDASITKKHLFASVHDAVTFALQHPRPVPDSPVSVTRL

SEQ ID NO: 75 - protein, 817 aa, Canis familiaris
MGAGAGAPPAPEGCVRSHSSAARGLASGRGRRLSVEEPRPGGGSPWVDKRFTEYSTYLTGANFPVRQRDTQALLPVPQAMELRKRDYHVERPLLNQEQLEELGCWTSATGTRQWRTWFQCSRARARALLFQHLPVLAWLPRYPLRDWLLGDLLAGLSVAIMQLPQGLAYALLAGLPPVFGLYSSFYPVFVYFLFGTSRHISVGTFAVMSVMVGSVTESLAPDENFLQAVNSTIDEATRDATRVELASTLSVLVGLFQVGLGLVRFGFVVTYLSEPLVRGYTTAASVQVFVSQLKYVFGLQLSSRSGPLSLIYTVLEVCSKLPQNVVGTVVTAVVAGVVLVLVKLLNDKLHRRLPLPIPGELLTLIGATAISYGVGLKHRFGVDIVGNIPAGLVPPAAPNPQLFASLVGYAFTIAVVGFAIAISLGKIFALRHGYRVDSNQELVALGLSNLIGGIFQCFPVSCSMSRSLVQEGAGGNTQVAGAVSSLFILIIIVKLGELFRDLPKAVLAAAIIVNLKGMLMQFTDIPSLWKSNRMDLLIWLVTFVATILLNLDIGLAVAVVFSLLLVVVRTQLPHYSVLGQVTDTDIYQDVAEYSEAREVPGVKVFRSSATMYFANAELYSDALKQRCGIDVDHLMSQKKKRLRKKEQKLKRLQKTLQKQTAASEGTSVSIHVNTSVRDMESNNVEDSKAQASTGNEVEDIAAGGQEDTKASNGSTLKALGLPQPHFHSLVLDLSALSFVDTVCIKSLKNIFRDFREIEVEVYLAACHTPVVTQLEAGHFFDASITKQHLFASVHDAVLFALQHPKSSPANPVLMTKL

SEQ ID NO: 76 - protein, 881 aa, Chlamydomonas reinhardtii
MAALSWQGIVAVTFTALAFVVMAADWVGPDITFTVLLAFLTAFDGQIVTVAKAAAGYGNTGLLTVVFLYWVAEGITQTGGLELIMNYVLGRSRSVHWALVRSMFPVMVLSAFLNNTPCVTFMIPILISWGRRCGVPIKKLLIPLSYAAVLGGTCTSIGTSTNLVIVGLQDARYAKSKQVDQAKFQIFDIAPYGVPYALWGFVFILLAQGFLLPGNSSRYAKDLLLAVRVLPSSSVVKKKLKDSGLLQQNGFDVTAIYRNGQLIKISDPSIVLDGGDILYVSGELDVVEFVGEEYGLALVNQEQELAAERPFGSGEEAVFSANGAAPYHKLVQAKLSKTSDLIGRTVREVSWQGRFGLIPVAIQRGNGREDGRLSDVVLAAGDVLLLDTTPFYDEDREDIKTNFDGKLHAVKDGAAKEFVIGVKVKKSAEVVGKTVSAAGLRGIPGLFVLSVDHADGTSVDSSDYLYKIQPDDTIWIAADVAAVGFLSKFPGLELVQQEQVDKTGTSILYRHLVQAAVSHKGPLVGKTVRDVRFRTLYNAAVVAVHRENARIPLKVQDIVLQGGDVLLISCHTNWADEHRHDKSFVLVQPVPDSSPPKRSRMIIGVLLATGMVLTQIIGGLKNKEYIHLWPCAVLTAALMLLTGCMNADQTRKAIMWDVYLTIAAAFGVSAALEGTGVAAKFANAIISIGKGAGGTGAALIAIYIATALLSELLTNNAAGAIMYPIAAIAGDALKITPKDTSVAIMLGASAGFVNPFSYQTNLMVYAAGNYSVREFAIVGAPFQVWLMIVAGFILVYRNQWHQVWIVSWICTAGIVLLPALYFLLPTRIQIKIDGFFERIAAVLNPKAALERRRSLRRQVSHTRTDDSGSSGSPLPAPKIVA

SEQ ID NO: 77 - protein, 883 aa, Chlamydomonas reinhardtii
MGFGWQGSVSIAFTALAFVVMAADWVGPDVTFTVLLAFLTAFDGQIVTVAKAAAGYGNTGLLTVIFLYWVAEGITQTGGLELIMNFVLGRSRSVHWALARSMFPVMCLSAFLNNTPCVTFMIPILISWGRRCGVPIKKLLIPLSYASVLGGTCTSIGTSTNLVIVGLQDARYTKAKQLDQAKFQIFDIAPYGVPYALWGFVFILLTQAFLLPGNSSRYAKDLLIAVRVLPSSSVAKKKLKDSGLLQQSGFSVSGIYRDGKYLSKPDPNWVLEPNDILYAAGEFDVVEFVGEEFGLGLVNADAETSAERPFTTGEESVFTPTGGAPYQKLVQATIAPTSDLIGRTVREVSWQGRFGLIPVAIQRGNGREDGRLNDVVLAAGDVLILDTTPFYDEEREDSKNNFAGKVRAVKDGAAKEFVVGVKVKKSSEVVNKTVSAAGLRGIPGLFVLSVDRADGSSVEASDYLYKIQPDDTIWIATDIGAVGFLAKFPGLELVQQEQVDKTGTSILYRHLVQAAVSHKGPIVGKTVRDVRFRTLYNAAVVAVHREGARVPLKVQDIVLQGGDVLLISCHTNWADEHRHDKSFVLLQPVPDSSPPKRSRMVIGVLLATGMVLTQIVGGLKSREYIHLWPAAVLTSALMLLTGCMNADQARKAIYWDVYLTIAAAFGVSAALEGTGVAASFANGIISIGKNLHSDGAALIAIYIATAMLSELLTNNAAGAIMYPIAAIAGDALKISPKETSVAIMLGASAGFINPFSYQCNLMVYAAGNYSVREFAIIGAPFQIWLMIVAGFILCYMKEWHQVWIVSWICTAGIVLLPALYFLLPTKVQLRIDAFFDRVAQTLNPKLIIERRNSIRRQASRTGSDGTGSSDSPRALGVPKVITA

SEQ ID NO: 78 - protein, 764 aa, Chlamydomonas reinhardtii
MKRNTSNVDTGGVPAPLNSTPSTRLIQNGYGDSKYETERMEFPFPEDPRYHPRDSVKGAWEKVKEDHHHRVATYNWVDWLAFFIPCVRWLRTYRRSYLLNDIVAGISVGFMVVPQGLSYANLAGLPSVYGLYGAFLPCIVYSLVGSSRQLAVGPVAVTSLLLGTKLKDILPEAAGISNPNIPGSPELDAVQEKYNRLAIQLAFLVACLYTGVGIFRLGFVTNFLSHAVIGGFTSGAAITIGLSQVKYILGISIPRQDRLQDQAKTYVDNMHNMKWQEFIMGTTFLFLLVLFKEVGKRSKRFKWLRPIGPLTVCIIGLCAVYVGNVQNKGIKIIGAIKAGLPAPTVSWWFPMPEISQLFPTAIVVMLVDLLESTSIARALARKNKYELHANQEIVGLGLANFAGAIFNCYTTTGSFSRSAVNNESGAKTGLACFITAWVVGFVLIFLTPVFAHLPYCTLGAIIVSSIVGLLEYEQAIYLWKVNKLDWLVWMASFLGVLFISVEIGLGIAIGLAILIVIYESAFPNTALVGRIPGTTIWRNIKQYPNAQLAPGLLVFRIDAPIYFANIQWIKERLEGFASAHRVWSQEHGVPLEYVILDFSPVTHIDATGLHTLETIVETLAGHGTQVVLANPSQEIIALMRRGGLFDMIGRDYVFITVNEAVTFCSRQMAERGYAVKEDNTSSYPHFGSRRTPGALPAPSSQLDSSPPTSVTESTSGTPAAGTYSSIGGAVPAVAGHTAAGNGGSHSPSAQPGVQLTTTGSQRQQ

SEQ ID NO: 79 - protein, 978 aa, Physcomitrella patens
MTRSMPLYRGEQEEMWFSHTESIKTTPSATTNAPLSDGIRIPRFHGVRGGPDPMHRNPDLRNVAVLLSCSVQGGEVLDLGVVPGAKPALYCWFGFMISSLLNCVMNCLFEFDFVESAENSGRELRRESDKMVQLGWESYLVLATLIAGLVVMAGDWVGPDFVFALMVGFLTACRVITVKESTEGFSQNGVLTVVILFVVAEGIGQTGGMEKALNLLLGKATSPFWAITRMFIPVAITSAFLNNTPIVALLIPIMIAWGRRNRISPKKLLIPLSYAAVFGGTLTQIGTSTNFVISSLQEKRYTQLKRPGDAKFGMFDITPYGIVYCIGGFLFTVIASHWLLPSDETKRHSDLLLVARVPPESPVANNTVREAGLKGMERLFLVAVERQGRVTHAVGPQYLLEPEDLLYFCGELEQAHFYSKAFSLELLTNEAISGSKRANFQGEKHPSALENGSCGSVEDSTLIMQASVRKGADIIGKTLDQIDFRKRFDVAVLGLKRGETHQPGPLSEMVVNANDVLVLLGDNEEVLQKPEVKAVFKDVEKLDEALEKEYLTGMKVTNRFKGVGKTVYDAGLRGINGLTLLAIDRQSGEHLKFIEDDTVVELGDTLWFAGGVQGVHFLLKISGLEHSQAPQVSKLRADILYRQLVKASVASESPLVGNTVREAHFRNKYDAVVLAIHRQGERLSMDVRDVKLRAGDVLLLDTGSNFGHRYRNDAAFSLISGVPESSPVKKSRMWVALFLGAAMIATQIVSSSIGGTELINLFTAGILTSGLMLLTRCLSADQARNSIDWRVYTTIAFAIAFSTCMEKSKLARAIADIFIKISESIGGMRASYVAIYIATALLSELVSNNAAAAIMYPIAADLGDALGVVPTRMSVVVMLGASAGFTLPYSYQTNLMVYAAGDYRFMEFAKFGLPCQCFMIITVILIFLLDNRIWVAVGLGFALMLVVLGWHLVWEFVPASIRSKFSPGRKEKTEKIEQ

SEQ ID NO: 80 - protein, 667 aa, Stylosanthes hamata
MSQRVSDQVMADVIAETRSNSSSHRHGGGGGGDDTTSLPYMHKVGTPPKQTLFQEIKHSFNETFFPDKPFGKFKDQSGFRKLELGLQYIFPILEWGRHYDLKKFRGDFIAGLTIASLCIPQDLAYAKLANLDPWYGLYSSFVAPLVYAFMGTSRDIAIGPVAVVSLLLGTLLSNEISNTKSHDYLRLAFTATFFAGVTQMLLGVCRLGFLIDFLSHAAIVGFMAGAAITIGLQQLKGLLGISNNNFTKKTDIISVMRSVWTHVHHGWNWETILIGLSFLIFLLITKYIAKKNKKLFWVSAISPMISVIVSTFFVYITRADKRGVSIVKHIKSGVNPSSANEIFFHGKYLGAGVRVGVVAGLVALTEAIAIGRTFAAMKDYALDGNKEMVAMGTMNIVGSLSSCYVTTGSFSRSAVNYMAGCKTAVSNIVMSIVVLLTLLVITPLFKYTPNAVLASIIIAAVVNLVNIEAMVLLWKIDKFDFVACMGAFFGVIFKSVEIGLLIAVAISFAKILLQVTRPRTAVLGKLPGTSVYRNIQQYPKAAQIPGMLIIRVDSAIYFSNSNYIKERILRWLIDEGAQRTESELPEIQHLITEMSPVPDIDTSGIHAFEELYKTLQKREVQLILANPGPVVIEKLHASKLTELIGEDKIFLTVADAVATYGPKTAAF

SEQ ID NO: 81 - protein, 653 aa, Arabidopsis thaliana
MSSRAHPVDGSPATDGGHVPMKPSPTRHKVGIPPKQNMFKDFMYTFKETFFHDDPLRDFKDQPKSKQFMLGLQSVFPVFDWGRNYTFKKFRGDLISGLTIASLCIPQDIGYAKLANLDPKYGLYSSFVPPLVYACMGSSRDIAIGPVAVVSLLLGTLLRAEIDPNTSPDEYLRLAFTATFFAGITEAALGFFRLGFLIDFLSHAAVVGFMGGAAITIALQQLKGFLGIKKFTKKTDIISVLESVFKAAHHGWNWQTILIGASFLTFLLTSKIIGKKSKKLFWVPAIAPLISVIVSTFFVYITRADKQGVQIVKHLDQGINPSSFHLIYFTGDNLAKGIRIGVVAGMVALTEAVAIGRTFAAMKDYQIDGNKEMVALGMMNVVGSMSSCYVATGSFSRSAVNFMAGCQTAVSNIIMSIVVLLTLLFLTPLFKYTPNAILAAIIINAVIPLIDIQAAILIFKVDKLDFIACIGAFFGVIFVSVEIGLLIAVSISFAKILLQVTRPRTAVLGNIPRTSVYRNIQQYPEATMVPGVLTIRVDSAIYFSNSNYVRERIQRWLHEEEEKVKAASLPRIQFLIIEMSPVTDIDTSGIHALEDLYKSLQKRDIQLILANPGPLVIGKLHLSHFADMLGQDNIYLTVADAVEACCPKLSNEV

SEQ ID NO: 82 - DNA, 2316 bp, Artificial Sequence
  Synthesized
atggttccacaaacagaaactaaagcaggtgctggattcaaagccggtgtaaaagactaccgtttaacatactacacacctgattacgtagtaagagatactgatattttagctgcattccgtatgactccacaactaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggcggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaacttgttgttgttgttgttgttaatcgggcggatctgcttatctggctggtgaccttcacggccaccatcttgctgaacctggaccttggcttggtggttgcggtcatcttctccctgctgctcgtggtggtccggacacagatgccccactactctgtcctggggcaggtgccagacacggatatttacagagatgtggcagagtactcagaggccaaggaagtccggggggtgaaggtcttccgctcctcggccaccgtgtactttgccaatgctgagttctacagtgatgcgctgaagcagaggtgtggtgtggatgtcgacttcctcatctcccagaagaagaaactgctcaagaagcaggagcagctgaagctgaagcaactgcagaaagaggagaagcttcggaaacaggctgcctcccccaagggcgcctcagtttccattaatgtcaacaccagccttgaagacatgaggagcaacaacgttgaggactgcaagatgatgcaggtgagctcaggagataagatggaagatgcaacagccaatggtcaagaagactccaaggccccagatgggtccacactgaaggccctgggcctgcctcagccagacttccacagcctcatcctggacctgggtgccctctcctttgtggacactgtgtgcctcaagagcctgaagaatattttccatgacttccgggagattgaggtggaggtgtacatggcggcctgccacagccctgtggtcagccagcttgaggctgggcacttcttcgatgcatccatcaccaagaagcatctctttgcctctgtccatgatgctgtcacctttgccctccaacacccgaggcctgtccccgacagccctgtttcggtcaccagactctga

SEQ ID NO: 83 - protein, 8 aa, Artificial Sequence
  Synthesized
VRAAAVXX

SEQ ID NO: 84 - DNA, 84 bp, Artificial Sequence
  Synthesized
gctgatcttaaacaacgttgtggtgttgatgttgattttttaattagtcaaaaaaaaaaacttcttaaagccatgggtggtggt

(19) United States
(12) Patent Application Publication
(10) Pub. No.: US 2013/0145495 A1
(43) Pub. Date: Jun. 6, 2013

(54) ENHANCED CARBON FIXATION IN PHOTOSYNTHETIC HOSTS

(71) Applicant: Richard T. Sayre, Webster Groves, MO (US)

(72) Inventor: Richard T. Sayre, Webster Groves, MO (US)

(73) Assignee: DONALD DANFORTH PLANT SCIENCE CENTER, St. Louis, MO (US)

(21) Appl. No.: 13/641,814

(22) PCT Filed: Apr. 25, 2011
(86) PCT No.: PCT/US11/33814

§ 371 Date: Feb. 5, 2013

Related U.S. Application Data
(60) Provisional application No. 61/327,717, filed on Apr. 25, 2010.

(51) Int. Cl. C12N 15/82

(52) U.S. Cl. CPC C12N 15/8243
USPC 800/278; 800/298; 800/296; 800/317.3; 800/320.3; 800/320.2; 800/320; 800/313; 800/317.2; 800/312; 800/322; 800/314; 800/317.4; 800/309; 800/306; 800/307; 800/323.2; 800/323; 800/323.3; 800/323.1; 127/65; 554/8; 127/34; 568/700

(57) ABSTRACT

This invention provides genetically modified photosynthetic organisms and methods and constructs for enhancing inorganic carbon fixation. A photosynthetic organism of the present invention comprises a RUBISCO fusion protein operatively coupled to a protein-protein interaction domain to enable the functional association of RUBISCO and carbonic anhydrase.

### CROSS REFERENCE TO RELATED APPLICATIONS

This application claims the benefit of U.S. provisional patent application No. 61/327,717 filed on Apr. 25, 2010, the entire contents of which are incorporated herein by reference.

### STATEMENT REGARDING FEDERALLY SPONSORED RESEARCH OR DEVELOPMENT

This invention was made with US government support. The government has certain rights in the invention.

### TECHNICAL FIELD

The present invention relates generally to methods and constructs for enhancing inorganic carbon fixation in photosynthetic organisms.

### BACKGROUND OF THE INVENTION

One of the major constraints limiting photosynthetic efficiency in algae and many crop plants is the competitive inhibition of CO₂ fixation by oxygen at the active site of Ribulose-1,5-bisphosphate carboxylase oxygenase (RubisCO). In plants such as these (“C3” plants), RubisCO catalyzes the primary fixation of CO₂ in the Calvin cycle leading to the production of two molecules of the 3-carbon product 3-phosphoglycerate (3-PGA). However in such C3 plants when oxygen is present, RubisCO can also accept oxygen producing 2-phosphoglycolate and 3-PGA. 2-phosphoglycolate is subsequently metabolized by the photorespiratory pathway leading to the loss of one previously fixed carbon as CO₂ and the generation of one molecule of 3-phosphoglycerate from two molecules of phosphoglycolate. Moreover the photorespiratory pathway not only losses previously fixed carbon as CO₂ it also reduces the regeneration of ribulose-1,5-bisphosphate (RuBP), the substrate for RubisCO. Overall, the competitive inhibition of CO₂ fixation by oxygen and the associated photorespiratory pathway reduce carbon fixation efficiency by 30% or more in C3 plants.

One way to reduce the competition of O₂ for CO₂ fixation is to increase the CO₂ concentration at the active site of RubisCO. Certain plants (“C4 plants”) effectively do this by pumping CO₂ into bundle sheath chloroplast. CO₂ is initially fixed by the cytoplasmic enzyme PEP carboxylase localized in the outer mesophyll cells and the resulting 4-carbon dicarboxylic acids are shunted to the bundle sheath cells where they are decarboxylated. Importantly, PEP carboxylase does not fix oxygen and has a higher K_{cat} for CO₂ than RubisCO. The CO₂ resulting from C4 acid decarboxylation elevates the CO₂ concentration around RubisCO (localized in bundle sheath cell chloroplasts) by 10-fold inhibiting the oxygenase reaction and photorespiration pathway.

Similarly, Cyanobacteria concentrate CO₂ near RubisCO to inhibit the RubisCO oxygenase reaction. In Cyanobacteria, bicarbonate, the non-gaseous hydrated form of CO₂ is pumped into the cell and concentrated in an energy-dependent manner. In the carboxysomes, which is a protein assemblage of carbonic anhydrase (CA), RubisCO activase and RubisCO, CA accelerates the conversion of bicarbonate to CO₂, the substrate for RubisCO. The close association of CA with RubisCO reduces the distance over which CO₂ must diffuse before contacting RubisCO, and effectively elevates the local CO₂ concentration around RubisCO inhibiting photorespiration. In some eukaryotic algae, a structure similar to the carboxysome, the chloroplastic pyrenoid body, carries out a similar function. Eukaryotic algae also pump and concentrate bicarbonate into the cell/chloroplast where it is fixed by RubisCO (reviewed by Spalding, (2008) J. Exp. Bot. 59(7): 1463-1473).

Carbonic anhydrases also play an important role in CO₂ fixation during photosynthesis, particularly in plants where a substantial portion of the dissolve inorganic carbon dioxide in cells is present as bicarbonate. This is attributable to the fact that under physiological conditions (i.e. at pH 8.0 and 25° C.), the spontaneous rate of conversion of bicarbonate into CO₂ is significantly slower than the rate of photosynthetic carbon fixation.

In fact it has been calculated that the spontaneous rate of conversion of bicarbonate to CO₂ is approximately 10,000 times slower (0.5×μM CO₂ s⁻¹) than the rate of photosynthetic CO₂ fixation (2.8 mM CO₂ s⁻¹) (Badger and Price, (1994) Annu. Rev. Plant Physiol. Plant Mol. Biol. 45: 369-92). Accordingly to enhance physiological rates of CO₂ fixation significantly more rapid rates of CO₂ production from bicarbonate are required.

Consistent with this conclusion, in C4 plants and algae, the presence of carbonic anhydrases has been demonstrated to have a substantial stimulatory effect on photosynthetic carbon fixation. This is due, at least in part to the fact that bicarbonate represents a substantial fraction of the total inorganic carbon in these cells. By comparison, in C3 plants, which do not pump bicarbonate or elevate internal CO₂ or bicarbonate concentrations, the expression of carbonic anhydrases alone would be predicted to have only a relatively slight impact on the overall rate of carbon fixation. CA (Badger and Price, (1994) Annu. Rev. Plant Physiol. Plant Mol. Biol. 45: 369-92).

The two different mechanisms of concentrating CO₂ that have evolved in C4 plants and Cyanobacteria, suggests that this approach to improving photosynthetic efficiency provides a significant selective advantage. Accordingly these well-studied photosynthetic systems have led researchers to consider the usefulness of such approaches in other species that lack these CO₂ concentrating mechanisms.

For example, currently there is a large effort to improve the yield of C3 plants such as rice by redesigning these plants at the cellular level to include C4 photosynthetic pathway and Kranz anatomy (See for example, Sage and Sage (2009) Plant and Cell Physiol. 50 (4):756-772; Zhu et al., (2010) J. Interg. Plant Biol. 52 (8):762-770; Furbank et al., (2009) Funct. Plant Biol. 36 (11):845-856; Weber and von Caemmerer (2010) Cum Opin. Plant Biol. 13 (3):257-265).

Additionally other strategies to improve carbon fixation rates include the use of directed evolution strategies to improve the kinetic properties of RubisCO by improving the rate of catalysis (Kcat) and/or the affinity for CO₂ (lower Km), as described by Stemmer et al. (US 2006/0117409 A1).

Another strategy has been to overexpress a carbonic anhydrase, an enzyme that catalyzes the conversion of bicarbonate to CO₂, as described by Edgerton et al. (US 2003/0233670 A1), or to fuse carbonic anhydrase to a RubisCO-binding protein in order to increase the local concentration of CO₂ at the active site of RubisCO, as described by Houtz (US 2009/0070901 A1).

Another strategy has been to express a bicarbonate transporter to raise levels of intracellular bicarbonate, as described by Kaplan et al. (US 2002/0042931 A1) and Edgerton et al. (US 2003/0233670 A1).

While these strategies have been to some extend effective, there remains the need for simple and reliable methods to increase improve carbon fixation rates across all photosynthetic organisms. The present invention, by exploiting the use of protein-protein interaction domains fused to RuBisCO, enables the formation of a functional complex between RubisCO and carbonic anhydrase. Surprisingly, the RubisCO fusion protein can still functionally associate with other large and small RuBisCO subunits to form a fully functional complex which is capable of high efficiency carbon fixation. Furthermore co-expression of a high activity carbonic anhydrase enables the local concentration of carbon dioxide in the immediate vicinity of RubisCO to be significantly increased, thereby decreasing competitive inhibition of CO₂ fixation by oxygen. As a result, the overall rate of carbon fixation is significantly increased.

### SUMMARY OF THE INVENTION

One embodiment includes a method of increasing the efficiency of carbon dioxide fixation in a photosynthetic organism, comprising the steps of:
i) providing a carbonic anhydrase enzyme which either a) inherently comprises a first protein-protein interaction domain partner, or b) is fused in frame to a first heterologous protein-protein domain partner;
ii) providing a fusion protein comprising a RubisCO protein subunit fused in frame to a second protein-protein interaction partner;
   wherein the first protein-protein interaction partner and said second protein-protein interaction partner, or the first heterologous protein-protein domain partner and the second protein-protein interaction partner can associate to form a protein complex; and
iii) expressing the carbonic anhydrase enzyme and the fusion protein in a chloroplast within the photosynthetic organism.

In some embodiments, the carbonic anhydrase enzyme comprises a sequence selected from Tables D2 to D5. In some embodiments, the second protein interaction domain partner is a STAS domain. In some embodiments, the carbonic anhydrase enzyme has a Kcat/Km of from about 1×10⁷ M⁻¹s⁻¹ to about 1.5×10⁸ M⁻¹s⁻¹. In some embodiments, the carbonic anhydrase is codon optimized for the photosynthetic organism. In some embodiments, the carbonic anhydrase is a human carbonic anhydrase II. In some embodiments, the carbonic anhydrase comprises SEQ. ID. No. 1. In some embodiments, the RubisCO protein subunit is the large subunit of RubisCO. In some embodiments, the RubisCO protein subunit is the small subunit of RubisCO.

In some embodiments, the second fusion protein comprises a RubisCO large protein subunit fused in frame to a STAS domain; wherein the method further includes a third fusion protein comprising a RubisCO small protein subunit fused in frame to a STAS domain; and wherein the method further comprises the step of expressing the first fusion protein, the second fusion protein, and the third fusion protein in a chloroplast within the photosynthetic organism.

Another embodiment includes a transgenic organism comprising:
i) a first nucleic acid sequence comprising a first heterologous polynucleotide sequence encoding a carbonic anhydrase enzyme which either a) inherently comprises a first protein-protein interaction domain partner, or b) is fused in frame to a first heterologous protein-protein domain partner;
ii) a second nucleic acid sequence comprising a second heterologous polynucleotide sequence encoding a RubisCO protein subunit operatively coupled to a second protein-protein interaction partner;
   wherein the first protein-protein interaction partner and said second protein-protein interaction partner, or the first heterologous protein-protein domain partner and the second protein-protein interaction partner can associate to form a protein complex.

In some embodiments, the carbonic anhydrase enzyme has a Kcat/Km of from about 1×10⁷ M⁻¹s⁻¹ to about 1.5×10⁸ M⁻¹s⁻¹. In some embodiments, the carbonic anhydrase is codon optimized for the photosynthetic organism. In some embodiments, the carbonic anhydrase is a human carbonic anhydrase II. In some embodiments, the carbonic anhydrase enzyme comprises a sequence selected from Tables D2 to D5. In some embodiments, the second protein interaction domain partner is a STAS domain. In some embodiments, the carbonic anhydrase comprises SEQ. ID. No. 1. In some embodiments, the first heterologous polynucleotide sequence is operatively coupled to a leaf specific promoter. In some embodiments, the first heterologous polynucleotide sequence is operatively coupled to a CAB1 promoter. In some embodiments, the second heterologous polynucleotide sequence is operatively coupled to a leaf specific promoter. In some embodiments, the second heterologous polynucleotide sequence is operatively coupled to a Cab1 promoter. In some embodiments, the RubisCO protein subunit is the large subunit of RubisCO. In some embodiments, the RubisCO protein subunit is the small subunit of RubisCO.

In some embodiments, the transgenic plant comprises; a) a second nucleic acid sequence comprising a second heterologous polynucleotide sequence encoding a RubisCO large protein subunit fused in frame to a STAS domain, and b) a third nucleic acid sequence comprising a third heterologous polynucleotide sequence encoding a RubisCO small protein subunit fused in frame to a STAS domain.

In some embodiments, the transgenic plant is a C3 plant. In some embodiments, the transgenic plant is selected from the from the group consisting of tobacco; cereals including wheat, rice and barley; beans including mung bean, kidney bean and pea; starch-storing plants including potato, cassaya and sweet potato; oil-storing plants including soybean, rape, sunflower and cotton plant; vegetables including tomato, cucumber, eggplant, carrot, hot pepper, Chinese cabbage, radish, water melon, cucumber, melon, crown daisy, spinach, cabbage and strawberry; garden plants including chrysanthemum, rose, carnation and petunia and Arabidopsis, and trees.

In some embodiments, the transgenic organism is an eukaryotic alga. In some embodiments, the transgenic plant is a C4 plant.

In some embodiments, the transgenic organism exhibits an increased growth rate and/or biomass of at least about any of: 10%, 12%, and 15%, as compared to a control host. In some embodiments, the transgenic organism exhibits an increased growth rate and/or biomass of at least about any of: 10%, 20%, 25%, 50%, 100%, and 200%, as compared to a control host.

In some embodiments, the transgenic organism exhibits a decrease in oxygenase activity catalyzed by RubisCO of at least about any of: 10%, 20%, 25%, 50%, 100%, and 200% as compared to a control host. In some embodiments, the transgenic organism exhibits an increase in carboxylase activity catalyzed by RubisCO of at least about any of: 10%, 20%, 25%, 50%, 100%, and 200%, as compared to a control host. In some embodiments, the transgenic organism exhibits an increase in the rate of carbon fixation of at least about any of: 10%, 20%, 25%, 50%, 100%, and 200%, as compared to a control host. In some embodiments, the transgenic organism exhibits an increase in the rate of oxygen evolution of at least about any of: 10%, 20%, 25%, 50%, 100%, and 200%, as compared to a control host. In some embodiments, the transgenic organism exhibits an increase in ATP levels of at least about any of: 10%, 20%, 25%, 50%, 100%, and 200%, as compared to a control host.

Another embodiment includes an expression vector comprising:
i) a first nucleic acid sequence comprising a first heterologous polynucleotide sequence encoding a carbonic anhydrase enzyme which either a) inherently comprises a first protein-protein interaction domain partner, or b) is fused in frame to a first heterologous protein-protein domain partner;
ii) a second nucleic acid sequence comprising a second heterologous polynucleotide sequence encoding a RubisCO protein subunit operatively coupled to a second protein-protein interaction partner;
   wherein the first protein-protein interaction partner and said second protein-protein interaction partner, or the first heterologous protein-protein domain partner and the second protein-protein interaction partner can associate to form a protein complex.

In some embodiments, the carbonic anhydrase is codon optimized for the photosynthetic organism. In some embodiments, the carbonic anhydrase is a human carbonic anhydrase II. In some embodiments, the carbonic anhydrase enzyme comprises a sequence selected from Tables D2 to D5. In some embodiments, the second protein interaction domain partner is a STAS domain. In some embodiments, the carbonic anhydrase comprises SEQ. ID. No. 1. In some embodiments, the first heterologous polynucleotide sequence is operatively coupled to a leaf specific promoter. In some embodiments, the first heterologous polynucleotide sequence is operatively coupled to a CAB1 promoter. In some embodiments the second heterologous polynucleotide sequence is operatively coupled to a leaf specific promoter. In some embodiments, the second heterologous polynucleotide sequence is operatively coupled to a CAB1 promoter. In some embodiments, the RubisCO protein subunit is the large subunit of RubisCO. In some embodiments, the RubisCO protein subunit is the small subunit of RubisCO.

Another embodiment includes method of producing a product from biomass from a photosynthetic organism comprising the steps of:
i) expressing a first nucleic acid sequence comprising a first heterologous polynucleotide sequence encoding a carbonic anhydrase enzyme which either a) inherently comprises a first protein-protein interaction domain partner, or b) is fused in frame to a first heterologous protein-protein domain partner;
ii) expressing a second nucleic acid sequence comprising a second heterologous polynucleotide sequence encoding a RubisCO protein subunit operatively coupled to a second protein-protein interaction partner;
wherein the first protein-protein interaction partner and said second protein-protein interaction partner, or the first heterologous protein-protein domain partner and the second protein-protein interaction partner can associate to form a protein complex;
iii) growing the transgenic organism; and
iv) harvesting the biomass.

In some embodiments, the product is selected from the group consisting of starches, oils, lipids, fatty acids, cellulose, carbohydrates, alcohols, sugars, nutraceuticals, pharmaceuticals and organic acids. In some embodiments, the transgenic organism is an eukaryotic algae. In some embodiments, the transgenic organism is a C3 plant. In some embodiments, the transgenic organism is a C4 plant.

### BRIEF DESCRIPTION OF THE DRAWINGS

FIG. 1 Shows an exemplary vector for creating an rbcL deletion host.

FIG. 2 Shows an exemplary expression vector for expressing a codon optimized human carbonic anhydrase (hs CAII) in the stroma of a chloroplast.

FIG. 3 Shows the nucleic acid, and translated amino acid sequence for an exemplary CA expression cassette for expression of a codon optimized human CA for expression in Chlamydomonas cells with ATP promoter and Rbc terminator.

FIG. 4 Shows the Relative colony growth of transgenic Chlamydomonas cells expressing Human CA-II and wild-type cells (—CA).

FIG. 5 Shows the Relative colony growth of transgenic Chlamydomonas cells expressing Human CA-II and wild-type cells (—CA) when grown at pH 8.5.

FIG. 6 depicts oxygen evolution from a photosynthetic host transformed with a CA and a control host.

FIG. 7 shows an exemplary RubisCO (RbcL) large subunit-STAS fusion protein construct.

FIG. 8 an exemplary expression vector for expressing a codon optimized human carbonic anhydrase (hs CAII) and RubisCO-STAS fusion proteins in the stroma of a chloroplast.

### DETAILED DESCRIPTION OF THE INVENTION

In order that the present disclosure may be more readily understood, certain terms are first defined. Additional definitions are set forth throughout the detailed description. As used herein and in the appended claims, the singular forms “a,” “an,” and “the,” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The terms “about” or “approximately” means within an acceptable error range for the particular value as determined by one of ordinary skill in the art, which will depend in part on how the value is measured or determined, i.e., the limitations of the measurement system. For example, “about” can mean within 1 or 2 standard deviations, from the mean value. Alternatively, “about” can mean plus or minus a range of up to 20%, preferably up to 10%, more preferably up to 5%.

As used herein, the terms “cell,” “cells,” “cell line,” “host cell,” and “host cells,” are used interchangeably and, encompass animal cells and include plant, invertebrate, non-mammalian vertebrate, insect, algal, and mammalian cells. All such designations include cell populations and progeny. Thus, the terms “transformants” and “transfectants” include the primary subject cell and cell lines derived therefrom without regard for the number of transfers.

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids can be defined where amino acids within a group exchange preferentially with each other, and therefore resemble each other most in their impact on the overall protein structure (Schulz, G. E. and R. H. Schirmer, Principles of Protein Structure, Springer-Verlag).

Examples of amino acid groups defined in this manner include: a “charged/polar group,” consisting of Glu, Asp, Asn, Gln, Lys, Arg and His; an “aromatic, or cyclic group,” consisting of Pro, Phe, Tyr and Trp; and an “aliphatic group” consisting of Gly, Ala, Val, Leu, Ile, Met, Ser, Thr and Cys.

Within each group, subgroups can also be identified, for example, the group of charged/polar amino acids can be sub-divided into the sub-groups consisting of the “positively-charged sub-group,” consisting of Lys, Arg and His; the negatively-charged sub-group,” consisting of Glu and Asp, and the “polar sub-group” consisting of Asn and Gln. The aromatic or cyclic group can be sub-divided into the sub-groups consisting of the “nitrogen ring sub-group,” consisting of Pro, His and Trp; and the “phenyl sub-group” consisting of Phe and Tyr. The aliphatic group can be sub-divided into the sub-groups consisting of the “large aliphatic non-polar sub-group,” consisting of Val, Leu and Ile; the “aliphatic slightly-polar sub-group,” consisting of Met, Ser, Thr and Cys; and the “small-residue sub-group,” consisting of Gly and Ala.

Examples of conservative mutations include substitutions of amino acids within the sub-groups above, for example, Lys for Arg and vice versa such that a positive charge can be maintained; Glu for Asp and vice versa such that a negative charge can be maintained; Ser for Thr such that a free —OH can be maintained; and Gln for Asn such that a free —NH₂ can be maintained.

The term “expression” as used herein refers to transcription and/or translation of a nucleotide sequence within a host cell. The level of expression of a desired product in a host cell may be determined on the basis of either the amount of corresponding mRNA that is present in the cell, or the amount of the desired polypeptide encoded by the selected sequence. For example, mRNA transcribed from a selected sequence can be quantified by Northern blot hybridization, ribonuclease RNA protection, in situ hybridization to cellular RNA or by PCR. Proteins encoded by a selected sequence can be quantified by various methods including, but not limited to, e.g., ELISA, Western blotting, radioimmunoassays, immunoprecipitation, assaying for the biological activity of the protein, or by immunostaining of the protein followed by FACS analysis.

“Expression control sequences” are regulatory sequences of nucleic acids, such as promoters, leaders, transit peptide sequences, enhancers, introns, recognition motifs for RNA, or DNA binding proteins, polyadenylation signals, terminators, internal ribosome entry sites (IRES) and the like, that have the ability to affect the transcription, targeting, or translation of a coding sequence in a host cell. Exemplary expression control sequences are described in Goeddel; Gene Expression Technology: Methods in Enzymology 185, Academic Press, San Diego, Calif. (1990).

A “gene” is a sequence of nucleotides which code for a functional gene product. Generally, a gene product is a functional protein. However, a gene product can also be another type of molecule in a cell, such as RNA (e.g., a tRNA or an rRNA). A gene may also comprise expression control sequences (i.e., non-coding) sequences as well as coding sequences and introns. The transcribed region of the gene may also include untranslated regions including introns, a 5′-untranslated region (5′-UTR) and a 3′-untranslated region (3′-UTR).

The term “heterologous” refers to a nucleic acid or protein which has been introduced into an organism (such as a plant, animal, or prokaryotic cell), or a nucleic acid molecule (such as chromosome, vector, or nucleic acid), which are derived from another source, or which are from the same source, but are located in a different (i.e. non native) context.

The term “homology” describes a mathematically based comparison of sequence similarities which is used to identify genes or proteins with similar functions or motifs. The nucleic acid and protein sequences of the present invention can be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention.

To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used.

The term “homologous” refers to the relationship between two proteins that possess a “common evolutionary origin”, including proteins from superfamilies (e.g., the immunoglobulin superfamily) in the same species of animal, as well as homologous proteins from different species of animal (for example, myosin light chain polypeptide, etc.; see Reeck et al., (1987) Cell, 50:667). Such proteins (and their encoding nucleic acids) have sequence homology, as reflected by their sequence similarity, whether in terms of percent identity or by the presence of specific residues or motifs and conserved positions.

As used herein, the term “increase” or the related terms “increased”, “enhance” or “enhanced” refers to a statistically significant increase. For the avoidance of doubt, the terms generally refer to at least a 10% increase in a given parameter, and can encompass at least a 20% increase, 30% increase, 40% increase, 50% increase, 60% increase, 70% increase, 80% increase, 90% increase, 95% increase, 97% increase, 99% or even a 100% increase over the control value.

The term “isolated,” when used to describe a protein or nucleic acid, means that the material has been identified and separated and/or recovered from a component of its natural environment. Contaminant components of its natural environment are materials that would typically interfere with research, diagnostic or therapeutic uses for the protein or nucleic acid, and may include enzymes, hormones, and other proteinaceous or non-proteinaceous solutes. In some embodiments, the protein or nucleic acid will be purified to at least 95% homogeneity as assessed by SDS-PAGE under non-reducing or reducing conditions using Coomassie blue or, preferably, silver stain. Isolated protein includes protein in situ within recombinant cells, since at least one component of the protein of interest's natural environment will not be present. Ordinarily, however, isolated proteins and nucleic acids will be prepared by at least one purification step.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs.

Optimal alignment of sequences for comparison can be conducted, for example, by the local homology algorithm of Smith & Waterman, by the homology alignment algorithms, by the search for similarity method or, by computerized implementations of these algorithms (GAP, BESTFIT, PASTA, and TFASTA in the GCG Wisconsin Package, available from Accelrys, Inc., San Diego, Calif., United States of America), or by visual inspection. See generally, (Altschul, S. F. et al., J. Molec. Biol. 215: 403-410 (1990) and Altschul et al. Nuc. Acids Res. 25: 3389-3402 (1997)).

One example of an algorithm that is suitable for determining percent sequence identity and sequence similarity is the BLAST algorithm, which is described in (Altschul, S., et al., NCBI NLM NIH Bethesda, Md. 20894; & Altschul, S., et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold.

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always; 0) and N (penalty score for mismatching residues; always; 0). For amino acid sequences, a scoring matrix is used to calculate the cumulative score. Extension of the word hits in each direction are halted when the −27 cumulative alignment score falls off by the quantity X from its maximum achieved value, the cumulative score goes to zero or below due to the accumulation of one or more negative-scoring residue alignments, or the end of either sequence is reached. The BLAST algorithm parameters W. T. and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

The terms “operably linked”, “operatively linked,” or “operatively coupled” as used interchangeably herein, refer to the positioning of two or more nucleotide sequences or sequence elements in a manner which permits them to function in their intended manner. In some embodiments, a nucleic acid molecule according to the invention includes one or more DNA elements capable of opening chromatin and/or maintaining chromatin in an open state operably linked to a nucleotide sequence encoding a recombinant protein. In other embodiments, a nucleic acid molecule may additionally include one or more DNA or RNA nucleotide sequences chosen from: (a) a nucleotide sequence capable of increasing translation; (b) a nucleotide sequence capable of increasing secretion of the recombinant protein outside a cell; (c) a nucleotide sequence capable of increasing the mRNA stability, and (d) a nucleotide sequence capable of binding a trans-acting factor to modulate transcription or translation, where such nucleotide sequences are operatively linked to a nucleotide sequence encoding a recombinant protein. Generally, but not necessarily, the nucleotide sequences that are operably linked are contiguous and, where necessary, in reading frame. However, although an operably linked DNA element capable of opening chromatin and/or maintaining chromatin in an open state is generally located upstream of a nucleotide sequence encoding a recombinant protein; it is not necessarily contiguous with it. Operable linking of various nucleotide sequences is accomplished by recombinant methods well known in the art, e.g. using PCR methodology, by ligation at suitable restrictions sites or by annealing. Synthetic oligonucleotide linkers or adaptors can be used in accord with conventional practice if suitable restriction sites are not present.

The terms “polynucleotide,” “nucleotide sequence” and “nucleic acid” are used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. A nucleic acid molecule can take many different forms, e.g., a gene or gene fragment, one or more exons, one or more introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. As used herein, a polynucleotide includes not only naturally occurring bases such as A, T, U, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Prokaryotic promoters contain Shine-Dalgarno sequences in addition to the −10 and −35 consensus sequences.

A large number of promoters, including constitutive, inducible and repressible promoters, from a variety of different sources are well known in the art. Representative sources include for example, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters active in plants include, for example nopaline synthase (nos) promoter and octopine synthase (ocs) promoters carried on tumor-inducing plasmids of Agrobacterium tumefaciens and the caulimovirus promoters such as the Cauliflower Mosaic Virus (CaMV) 19S or 35S promoter (U.S. Pat. No. 5,352,605), CaMV 35S promoter with a duplicated enhancer (U.S. Pat. Nos. 5,164,316; 5,196,525; 5,322,938; 5,359,142; and 5,424,200), the Figwort Mosaic Virus (FMV) 35S promoter (U.S. Pat. No. 5,378,619), and the cassaya vein mosaic virus promoter (U.S. Pat. No. 7,601,885). These promoters and numerous others have been used in the creation of constructs for transgene expression in plants or plant cells. Other useful promoters are described, for example, in U.S. Pat. Nos. 5,391,725; 5,428,147; 5,447,858; 5,608,144; 5,614,399; 5,633,441; 6,232,526; and 5,633,435, all of which are incorporated herein by reference.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell. Methods for purification are well-known in the art. As used herein, the term “substantially free” is used operationally, in the context of analytical testing of the material. Preferably, purified material substantially free of contaminants is at least 50% pure; more preferably, at least 75% pure, and more preferably still at least 95% pure. Purity can be evaluated by chromatography, gel electrophoresis, immunoassay, composition analysis, biological assay, and other methods known in the art. The term “substantially pure” indicates the highest degree of purity, which can be achieved using conventional purification techniques known in the art.

The term “sequence similarity” refers to the degree of identity or correspondence between nucleic acid or amino acid sequences that may or may not share a common evolutionary origin. However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 85%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

In particular embodiments of the invention, two amino acid sequences are “substantially homologous” or “substantially similar” when greater than 90% of the amino acid residues are identical. Two sequences are functionally identical when greater than about 95% of the amino acid residues are similar. Preferably the similar or homologous polypeptide sequences are identified by alignment using, for example, the GCG (Genetics Computer Group, Version 7, Madison, Wis.) pileup program, or using any of the programs and algorithms described above. The program may use the local homology algorithm of Smith and Waterman with the default values: Gap creation penalty=−(1+1/k), k being the gap extension number, Average match=1, Average mismatch=−0.333.

As used herein, a “transgenic plant” is one whose genome has been altered by the incorporation of heterologous genetic material, e.g. by transformation as described herein. The term “transgenic plant” is used to refer to the plant produced from an original transformation event, or progeny from later generations or crosses of a transgenic plant, so long as the progeny contains the heterologous genetic material in its genome.

The term “transformation” or “transfection” refers to the transfer of one or more nucleic acid molecules into a host cell or organism. Methods of introducing nucleic acid molecules into host cells include, for instance, calcium phosphate transfection, DEAE-dextran mediated transfection, microinjection, cationic lipid-mediated transfection, electroporation, scrape loading, ballistic introduction, or infection with viruses or other infectious agents.

“Transformed”, “transduced”, or “transgenic”, in the context of a cell, refers to a host cell or organism into which a recombinant or heterologous nucleic acid molecule (e.g., one or more DNA constructs or RNA, or siRNA counterparts) has been introduced. The nucleic acid molecule can be stably expressed (i.e. maintained in a functional form in the cell for longer than about three months) or non-stably maintained in a functional form in the cell for less than three months i.e. is transiently expressed. For example, “transformed,” “transformant,” and “transgenic” cells have been through the transformation process and contain foreign nucleic acid. The term “untransformed” refers to cells that have not been through the transformation process.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Buchanan et al., Biochemistry and Molecular Biology of Plants, Courier Companies, USA, 2000; Mild and Iyer, Plant Metabolism, 2^{nd} Ed. D. T. Dennis, D H Turpin, D D Lefebrve, D G Layzell (eds) Addison Wesly, Langgmans Ltd. London (1997); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein.

The publications discussed above are provided solely for their disclosure before the filing date of the present application. Nothing herein is to be construed as an admission that the invention is not entitled to antedate such disclosure by virtue of prior invention. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and reference.

### I. Overview

The present invention relates to transgenic strategies for enhancing carbon fixation in a photosynthetic organism by concentrating CO₂ in the microenvironment of RubisCO. As detailed herein, the co-expression of Carbonic anhydrase with RubisCo within the chloroplasts of plants results in an increase in the carboxylase activity and/or decrease in oxygenase activity of RubisCO.

In certain embodiments, the RubsiCO is fused to a protein-protein interaction domain that mediated the formation of a complex of RubisCO and carbonic anhydrase that results in a significant enhance in carbon dioxide fixation rate and biomass yield.

### II. Carbonic Anhydrase

Carbonic anhydrases (CA) are zinc-containing metalo-enzymes found ubiquitously throughout nature in prokaryotes and eukaryotes. Carbonic anhydrases catalyses the reversible hydration of CO₂ to bicarbonate and play a central role in controlling pH balance and inorganic carbon sequestration and flux in many organisms. The carbonic anhydrases are a diverse group of proteins but can be divided into four evolutionary distinct classes; the α-CAs (found in vertebrates, bacteria, algae and cytoplasm of green plants); β-CAs (found in bacteria, algae and chloroplasts); —CAs (found in archaea and bacteria); and δ-CAs (found in marine diatoms). (Supuran, (2008) Curr. Pharma. Des. 14: 603-614).

There are approximately 16 different classes of α-CAs found in mammals (See Table D1), and these, as well as any of the homologous genes from other organisms are potentially suitable for use in any of the claimed methods, DNA constructs, and transgenic plants.

TABLE D1

| | | | Kcat/ | | | |
|---|---|---|---|---|---|---|
| | Kcat | Km | Km | Ki | Subcellular | Tissue/organ |
| Isoenzyme | (s⁻¹) | (mM) | (M⁻¹s⁻¹) | (nM) | localization | localization |
| hCAI | 2 × 10⁵ | 4.0 | 5.0 × 10⁷ | 250 | cytosol | E, GI |
| hCAII | 1.4 × 10⁶ | 9.3 | 1.5 × 10⁸ | 12 | cytosol | E, eye, GI, BO, |
| | | | | | | K, L, T, B |
| hCAIII | 1.0 × 10⁴ | 33.3 | 3.0 × 10⁵ | 2 × 10⁵ | cytosol | SM, A |
| hCAIV | 1.0 × 10⁶ | 21.5 | 5.1 × 10⁷ | 74 | membrane | K, L, P, B, C, H |
| hCAVA | 2.9 × 10⁵ | 10.0 | 2.9 × 10⁷ | 63 | mitochondria | Li |
| hCAVB | 9.5 × 10⁵ | 9.7 | 9.8 × 10⁷ | 54 | mitochondria | H, SM, P, K, |
| | | | | | | SC, GI |
| hCAVI | 3.4 × 10⁵ | 6.9 | 4.9 × 10⁷ | 11 | secreted | G |
| hCAVII | 9.5 × 10⁵ | 11.4 | 8.3 × 10⁷ | 2.5 | cytosol | CNS |
| hCAVIII | | | | | cytosol | CNS |
| hCAIX | 3.8 × 10⁵ | 6.9 | 5.5 × 10⁷ | 25 | transmembrane | TU, GI |
| hCAX | | | | | cytosol | CNS |
| hCAXI | | | | | cytosol | CNS |
| hCAXII | 4.2 × 10⁵ | 12.0 | 3.5 × 10⁷ | 5.7 | transmembrane | R, I, RE, eye, |
| | | | | | | TU |
| hCAXIII | 1.5 × 10⁵ | 13.8 | 1.1 × 10⁷ | 16 | cytosol | K, B, L, GI, RE |
| hCAXIV | 3.1 × 10⁵ | 7.9 | 3.9 × 10⁷ | 41 | transmembrane | K, B, L |
| hCAXV | 4.7 × 10⁵ | 14.2 | 3.3 × 10⁷ | 72 | membrane | K |

H = Human; M = Mouse; hCAVIII, X, and XI are devoid of catalytic activity. E = Erthrocyes; GI = GI tract; BO = Bone osteoclasts; K = kidney, L = Lung; T = testis; B = brain; SM = skeletal muscle; A = Adipocytes; P = pancreas; C = colon; H = heart; Li = liver; SC = spinal cord; G = salivary and mammary gland; R = renal; I = intestinal; TU = tumors, RE = Reproductive

In any of these methods, DNA constructs, and transgenic organisms, the terms “CA” or “carbonic anhydrase” refers to all naturally-occurring and synthetic genes encoding carbonic anhydrase. In one aspect, the carbonic anhydrase gene is from a plant. In one aspect the carbonic anhydrase is from a mammal. In one aspect, the carbonic anhydrase is from a human. In one aspect the carbonic anhydrase can bind to a STAS domain. In one aspect the carbonic anhydrase is naturally expressed within the cytosol or is secreted. In one aspect the carbonic anhydrase has a Kcat/Km of greater than about 1×10⁷ M⁻¹s⁻¹. In one aspect the carbonic anhydrase has a Kcat/Km of greater than about 2×10⁷ M⁻¹s⁻¹. In one aspect the carbonic anhydrase has a Kcat/Km of greater than about 5×10⁷ M⁻¹s⁻¹. In one aspect the carbonic anhydrase has a Kcat/Km of greater than about 1×10⁸ M⁻¹s⁻¹. Representative species, Gene bank accession numbers, and amino acid sequences for various species of suitable CA genes are listed below in Tables D2-D4.

TABLE D2
Exemplary Type II Carbonic Anhydrases

| | | Accession | SEQ. ID |
|---|---|---|---|
| Organism | Sequence | Number | NO |
| Human | MSHHWGYGKH NGPEHWHKDF PIAKGERQSP | NP_000058.1 | SEQ. ID. |
| | VDIDTHTAKY DPSLKPLSVS YDQATSLRIL | | NO. 1 |
| | NNGHAFNVEF DDSQDKAVLK GGPLDGTYRL | | |
| | IQFHFHWGSL DGQGSEHTVD KKKYAAELHL | | |
| | VHWNTKYGDF GKAVQQPDGL AVLGIFLKVG | | |
| | SAKPGLQKVV DVLDSIKTKG KSADFTNFDP | | |
| | RGLLPESLDY WTYPGSLTTP PLLECVTWIV | | |
| | LKEPISVSSE QVLKFRKLNF NGEGEPEELM | | |
| | VDNWRPAQPL KNRQIKASFK | | |
| Macaca | MSHHWGYGKH NGPEHWHKDF PIAKGQRQSP | BAE91302.1 | SEQ. ID. |
| fascicularis | VDIDTHTAKY DPSLKPLSVS YDQATSLRIL | | NO. 2 |
| (crab-eating | NNGHSFNVEF DDSQDKAVIK GGPLDGTYRL | | |
| macaque) | IQFHFHWGSL DGQGSEHTVD KKKYAAELHL | | |
| | VHWNTKYGDF GKAVQQPDGL AVLGIFLKVG | | |
| | SAKPGLQKVV DVLDSIKTKG KSADFTNFDP | | |
| | RGLLPESLDY WTYPGSLTTP PLLECVTWIV | | |
| | LKEPISVSSE QMSKFRKLNF NGEGEPEELM | | |
| | VDNWRPAQPL KNRQIKASFK | | |
| Pan troglodytes | MSHHWGYGKH NGPEHWHKDF PIAKGERQSP | NP_001181853 | SEQ. ID. |
| | VDIDTHTAKY DPSLKPLSVS YGQATSLRIL | | NO.3 |
| | NNGHAFNVEF DDSQDKAVLK GGPLDGTYRL | | |
| | IQFHFHWGSL DGQGSEHTVD KKKYAAELHL | | |
| | VHWNTKYGDF GKAVQQPDGL AVLGIFLKVG | | |
| | SAKPGLQKVV DVLDSIKTKG KSADFTNFDP | | |
| | HGLLPESLDY WTYPGSLTTP PLLECVTWIV | | |
| | LKEPISVSSE QMLKFRKLNF NGEGEPEELM | | |
| | VDNWRPAQPL KNRQIKASFK | | |
| Macaca mulatta | MSHHWGYGKH NGPEHWHKDF PIAKGQRQSP | NP_001182346 | SEQ. ID. |
| | VDINTHTAKY DPSLKPLSVS YDQATSLRIL | | NO. 4 |
| | NNGHSFNVEF DDSQDKAVIK GGPLDGTYRL | | |
| | IQFHFHWGSL DGQGSEHTVD KKKYAAELHL | | |
| | VHWNTKYGDF GKAVQQPDGL AVLGIFLKVG | | |
| | SAKPGLQKVV DVLDSIKTKG KSADFTNFDP | | |
| | RGLLPESLDY WTYPGSLTTP PLLECVTWIV | | |
| | LKEPISVSSE QMSKFRKLNF NGEGEPEELM | | |
| | VDNWRPAQPL KNRQIKASFK | | |
| Pongo abelii | MSHHWGYGKH NGPEHWHKDF PIAKGERQSP | XP_002819286 | SEQ. ID. |
| | VDIDTHTAKY DPSLKPLSVC YDQATSLRIL | | NO. 5 |
| | NNGHSFNVEF DDSQDKAVLK GGPLDGTYRL | | |
| | IQFHFHWGSL DGQGSEHTVD KKKYAAELHL | | |
| | VHWNTKYGDF GKAVQQPDGL AVLGIFLKVG | | |
| | SAKPGLQKVV DVLDSIKTKG KCADFTNFDP | | |
| | RGLLPASLDY WTYPGSLTTP PLLECVTWIV | | |
| | LKEPISVSSE QMLKFRKLNF NGEGEPEELM | | |
| | VDNWRPAQPL KKRQIKASFK | | |
| Callithrix | MSHHWGYGKH NGPEHWHKDF PIAKGERQSP | XP_002759086 | SEQ. ID. |
| jacchus | VDIDTHTAKY DPSLKPLSVS YDQATSWRIL | | NO. 6 |
| | NNGHSFNVEF DDSQDKAVLK GGPLDGTYRL | | |
| | IQFHFHWGST DGQGSEHTVD KKKYAAELHL | | |
| | VHWNTKYGDF GKAAQQPDGL AVLGIFLKVG | | |
| | SAKPGLQKVV DVLDSIKTKG KSADFTNFDP | | |
| | RGLLPESLDY WTYPGSLTTP PLLESVTWIV | | |
| | LKEPISVSSE QILKFRKLNF SGEGEPEELM | | |
| | VDNWRPAQPL KNRQIKASFK | | |
| Lemur catta | MSHHWGYGKH NGPEHWHKDF PIAKGERQSP | ADD83028 | SEQ. ID. |
| | VDINTGAAKH DPSLKPLSVY YEQATSRRIL | | NO. 7 |
| | NNGHSFNVEF DDSQDKAVLK GGPLDGTYRL | | |
| | IQFHFHWGSL DGQGSEHTVD KKKYAAELHL | | |
| | VHWNTKYGDF GKAVQQPDGL AVLGIFLKVG | | |
| | SAKPGLQKVV DVLDSIKTKG KSADFTNFDP | | |
| | RGLLPESLDY WTYLGSLTTP PLLECVTWIV | | |
| | LKEPISVSSE QMMKFRKLSF SGEGEPEELM | | |
| | VDNWRPAQPL KNRQIKASFK | | |
| Ailuropoda | MAHHWGYGKH NGPEHWYKDF PIAKGQRQSP | XP_002916939 | SEQ. ID. |
| melanoleuca | VDIDTKAAIH DPALKALCPT YEQAVSQRVI | | NO. 8 |
| | NNGHSFNVEF DDSQDNAVLK GGPLTGTYRL | | |
| | IQFHFHWGSS DGQGSEHTVD KKKYAAELHL | | |
| | VHWNTKYGDF GKAVQQPDGL AVLGIFLKIG | | |
| | DARPGLQKVL DALDSIKTKG KSADFTNFDP | | |
| | RGLLPESLDY WTYPGSLTTP PLLECVTWIV | | |
| | LKEPISVSSE QMLKFRRLNF NKEGEPEELM | | |
| | VDNWRPAQPL HNRQINASFK | | |
| Equus caballus | MSHHWGYGQH NGPKHWHKDF PIAKGQRQSP | XP_001488540 | SEQ. ID. |
| | VDIDTKAAVH DAALKPLAVH YEQATSRRIV | | NO. 9 |
| | NNGHSFNVEF DDSQDKAVLQ GGPLTGTYRL | | |
| | IQFHFHWGSS DGQGSEHTVD KKKYAAELHL | | |
| | VHWNTKYGDF GKAVQQPDGL AVVGVFLKVG | | |
| | GAKPGLQKVL DVLDSIKTKG KSADFTNFDP | | |
| | RGLLPESLDY WTYPGSLTTP PLLECVTWIV | | |
| | LREPISVSSE QLLKFRSLNF NAEGKPEDPM | | |
| | VDNWRPAQPL NSRQIRASFK | | |
| Canis lupus | MAHHWGYAKH NGPEHWHKDF PIAKGERQSP | NP_001138642 | SEQ. ID. |
| familiaris | VDIDTKAAVH DPALKSLCPC YDQAVSQRII | | NO. 10 |
| | NNGHSFNVEF DDSQDKTVLK GGPLTGTYRL | | |
| | IQFHFHWGSS DGQGSEHTVD KKKYAAELHL | | |
| | VHWNTKYGEF GKAVQQPDGL AVLGIFLKIG | | |
| | GANPGLQKIL DALDSIKTKG KSADFTNFDP | | |
| | RGLLPESLDY WTYPGSLTTP PLLECVTWIV | | |
| | LKEPISVSSE QMLKFRKLNF NKEGEPEELM | | |
| | MDNWRPAQPL HSRQINASFK | | |
| Oryctolagus | MSHHWGYGKH NGPEHWHKDF PIANGERQSP | NP_001182637 | SEQ. ID. |
| cuniculus | IDIDTNAAKH DPSLKPLRVC YEHPISRRII | | NO. 11 |
| | NNGHSFNVEF DDSHDKTVLK EGPLEGTYRL | | |
| | IQFHFHWGSS DGQGSEHTVN KKKYAAELHL | | |
| | VHWNTKYGDF GKAVKHPDGL AVLGIFLKIG | | |
| | SATPGLQKVV DTLSSIKTKG KSVDFTDFDP | | |
| | RGLLPESLDY WTYPGSLTTP PLLECVTWIV | | |
| | LKEPITVSSE QMLKFRNLNF NKEAEPEEPM | | |
| | VDNWRPTQPL KGRQVKASFV | | |
| Ailuropoda | GPEHWYKDFP IAKGQRQSPV DIDTKAAIHD | EFB24165 | SEQ. ID. |
| melanoleuca | PALKALCPTY EQAVSQRVIN NGHSFNVEFD | | NO. 12 |
| | DSQDNAVLKG GPLTGTYRLI QFHFHWGSSD | | |
| | GQGSEHTVDK KKYAAELHLV HWNTKYGDFG | | |
| | KAVQQPDGLA VLGIFLKIGD ARPGLQKVLD | | |
| | ALDSIKTKGK SADFTNFDPR GLLPESLDYW | | |
| | TYPGSLTTPP LLECVTWIVL KEPISVSSEQ | | |
| | MLKFRRLNFN KEGEPEELMV DNWRPAQPLH | | |
| | NRQINASFK | | |
| Sus scrofa | MSHHWGYDKH NGPEHWHKDF PIAKGDRQSP | XP_001927840.1 | SEQ. ID. |
| | VDINTSTAVH DPALKPLSLC YEQATSQRIV | | NO. 13 |
| | NNGHSFNVEF DSSQDKGVLE GGPLAGTYRL | | |
| | IQFHFHWGSS DGQGSEHTVD KKKYAAELHL | | |
| | VHWNTKYKDF GEAAQQPDGL AVLGVFLKIG | | |
| | NAQPGLQKIV DVLDSIKTKG KSVEFTGFDP | | |
| | RDLLPGSLDY WTYPGSLTTP PLLESVTWIV | | |
| | LREPISVSSG QMMKFRTLNF NKEGEPEHPM | | |
| | VDNWRPTQPL KNRQIRASFQ | | |
| Callithrix | MSHHWGYGKH NGPEHWHKDF PIAKGERQSP | XP_002759087 | SEQ. ID. |
| jacchus | VDIDTHTAKY DPSLKPLSVS YDQATSWRIL | | NO. 14 |
| | NNGHSFNVEF DDSQDKAVLK GGPLDGTYRL | | |
| | IQLHLVHWNT KYGDFGKAAQ QPDGLAVLGI | | |
| | FLKVGSAKPG LQKVVDVLDS IKTKGKSADF | | |
| | TNFDPRGLLP ESLDYWTYPG SLTTPPLLES | | |
| | VTWIVLKEPI SVSSEQILKF RKLNFSGEGE | | |
| | PEELMVDNWR PAQPLKNRQI KASFK | | |
| Mus musculus | MSHHWGYSKH NGPENWHKDF PIANGDRQSP | NP_033931 | SEQ. ID. |
| | VDIDTATAQH DPALQPLLIS YDKAASKSIV | | NO. 15 |
| | NNGHSFNVEF DDSQDNAVLK GGPLSDSYRL | | |
| | IQFHFHWGSS DGQGSEHTVN KKKYAAELHL | | |
| | VHWNTKYGDF GKAVQQPDGL AVLGIFLKIG | | |
| | PASQGLQKVL EALHSIKTKG KRAAFANFDP | | |
| | CSLLPGNLDY WTYPGSLTTP PLLECVTWIV | | |
| | LREPITVSSE QMSHFRTLNF NEEGDAEEAM | | |
| | VDNWRPAQPL KNRKIKASFK | | |
| Bos taurus | MSHHWGYGKH NGPEHWHKDF PIANGERQSP | NP_848667 | SEQ. ID. |
| | VDIDTKAVVQ DPALKPLALV YGEATSRRMV | | NO. 16 |
| | NNGHSFNVEY DDSQDKAVLK DGPLTGTYRL | | |
| | VQFHFHWGSS DDQGSEHTVD RKKYAAELHL | | |
| | VHWNTKYGDF GTAAQQPDGL AVVGVFLKVG | | |
| | DANPALQKVL DALDSIKTKG KSTDFPNFDP | | |
| | GSLLPNVLDY WTYPGSLTTP PLLESVTWIV | | |
| | LKEPISVSSQ QMLKFRTLNF NAEGEPELLM | | |
| | LANWRPAQPL KNRQVRGFPK | | |
| Oryctolagus | GKHNGPEHWH KDFPIANGER QSPIDIDTNA | AAA80531 | SEQ. ID. |
| cuniculus | AKHDPSLKPL RVCYEHPISR RIINNGHSFN | | NO. 17 |
| | VEFDDSHDKT VLKEGPLEGT YRLIQFHFHW | | |
| | GSSDGQGSEH TVNKKKYAAE LHLVHWNTKY | | |
| | GDFGKAVKHP DGLAVLGIFL KIGSATPGLQ | | |
| | KVVDTLSSIK TKGKSVDFTD FDPRGLLPES | | |
| | LDYWTYPGSL TTPPLLECVT WIVLKEPITV | | |
| | SSEQMLKFRN LNFNKEAEPE EP | | |
| Rattus | MSHHWGYSKS NGPENWHKEF PIANGDRQSP | NP062164 | SEQ. ID. |
| norvegicus | VDIDTGTAQH DPSLQPLLIC YDKVASKSIV | | NO. 18 |
| | NNGHSFNVEF DDSQDFAVLK EGPLSGSYRL | | |
| | IQFHFHWGSS DGQGSEHTVN KKKYAAELHL | | |
| | VHWNTKYGDF GKAVQHPDGL AVLGIFLKIG | | |
| | PASQGLQKIT EALHSIKTKG KRAAFANFDP | | |
| | CSLLPGNLDY WTYPGSLTTP PLLECVTWIV | | |
| | LKEPITVSSE QMSHFRKLNF NSEGEAEELM | | |
| | VDNWRPAQPL KNRKIKASFK | | |

TABLE D3
Exemplary Type VII Carbonic Anhydrases

| | | Accession | SEQ. |
|---|---|---|---|
| Organism | Sequence | Number | ID. NO |
| Human | MSLSITNNGH SVQVDFNDSD DRTVVTGGPL | | SEQ. ID. |
| | EGPYRLKQFH FHWGKKHDVG SEHTVDGKSF | | NO. 19 |
| | PSELHLVHWN AKKYSTFGEA ASAPDGLAVV | | |
| | GVFLETGDEH PSMNRLTDAL YMVRFKGTKA | | |
| | QFSCFNPKCL LPASRHYWTY PGSLTTPPLS | | |
| | ESVTWIVLRE PICISERQMG KFRSLLFTSE | | |
| | DDERIHMVNN FRPPQPLKGR VVKASFRA | | |
| Pongo | MTGHHGWGYG QDDGPSHWHK LYPIAQGDRQ | XP_002826555 | SEQ. ID. |
| abelii | SPINIISSQA VYSPSLQPLE LSYEACMSLS | | NO. 20 |
| | ITNNGHSVQV DFNDSDDRTV VTGGPLEGPY | | |
| | RLKQFHFHWG KKHDVGSEHT VDGKSFPSEL | | |
| | HLVHWNAKKY STFGEAASAP DGLAVVGVFL | | |
| | ETGDEHPSMN RLTDALYMVR FKGTKAQFSC | | |
| | FNPKSLLPAS RHYWTYPGSL TTPPLSESVT | | |
| | WIVLREPICI SERQMGKFRS LLFTSEDDER | | |
| | IHMVNNFRPP QPLKGRVVKA SFRA | | |
| Pan | MEFGLSPELS PSRCFKRLLR GSERGRSRSP | XP_001143159.1 | SEQ. ID. |
| troglodytes | NERTEPTGQV HGCGDGSGMT GHHGWGYGQD | | NO. 21 |
| | DGPSHWHKLY PIAQGDRQSP INIISSQAVY | | |
| | SPSLQPLELS YEACMSLSIT NNGHSVQVDF | | |
| | NDSDDRTVVT GGPLEGPYRL KQFHFHWGKK | | |
| | HDVGSEHTVD GKSFPSELHL VHWNAKKYST | | |
| | FGEAASAPDG LAVVGVFLET GDEHPSMNRL | | |
| | TDALYMVRFK GTKAQFSCFN PKCLLPASRH | | |
| | YWTYPGSLTT PPLSESVTWI VLREPICISE | | |
| | RQMRKFRSLL FTSEDDERIH MVNNFRPPQP | | |
| | LKGRVVKASF RA | | |
| Callithrix | MTGHHGWGYG QDDGPSHWHK LYPIAQGDRQ | XP_002761099 | SEQ. ID. |
| jacchus | SPINIISSQA VYSPSLQPLE LSYEACMSLS | | NO.22 |
| | ITNNGHSVQV DFNDSDDRTV VTGGPLEGPY | | |
| | RLKQFHFHWG KKHDVGSEHT VDGKSFPSEL | | |
| | HLVHWNAKKY STFGEAASAP DGLAVVGVFL | | |
| | ETGDEHPSMN RLTDALYMVR FKGTKAQFSC | | |
| | FNPKCLLPAS WHYWTYPGSL TTPPLSESVT | | |
| | WIVLREPICI SERQMGKFRS LLFTSEDDER | | |
| | VHMVNNFRPP QPLKGRVVKA SFRA | | |
| Ailuropoda | GPSQWHKLYP IAQGDRQSPI NIVSSQAVYS | EFB15849 | SEQ. ID. |
| melanoleuca | PSLKPLELSY EACISLSIAN NGHSVQVDFN | | NO. 23 |
| | DSDDRTVVTG GPLDGPYRLK QFHFHWGKKH | | |
| | SVGSEHTVDG KSFPSELHLV HWNAKKYSTF | | |
| | GEAASAPDGL AVVGVFLETG DEHPSMNRLT | | |
| | DALYMVRFKG TKAQFSCFNP KCLLPASRHY | | |
| | WTYPGSLTTP PLSESVTWIV LREPISISER | | |
| | QMEKFRSLLF TSEDDERIHM VNNFRPPQPL | | |
| | KGRVVKASFR A | | |
| Canis | MTGHHCWGYG QNDEIQASLS PSLSTPAGPS | XP_546892 | SEQ. ID. |
| familiaris | QWHKLYPIAQ GDRQSPINIV SSQAVYSPSL | | NO. 24 |
| | KPLELSYEAC ISLSITNNGH SVQVDFNDSD | | |
| | DRTAVTGGPL DGPYRLKQLH FHWGKKHSVG | | |
| | SEHTVDGKSF PSELHLVHWN AKKYSTFGEA | | |
| | ASAPDGLAVV GIFLETGDEH PSMNRLTDAL | | |
| | YMVRFKGTKA QFSCFNPKCL LPASRHYWTY | | |
| | PGSLTTPPLS ESVTWIVLRE PISISERQME | | |
| | KFRSLLFTSE EDERIHMVNN FRPPQPLKGR | | |
| | VVKASFRA | | |
| Bos taurus | MTGHHGWGYG QNDGPSHWHK LYPIAQGDRQ | XP_002694851 | SEQ. ID. |
| | SPINIVSSQA VYSPSLKPLE ISYESCTSLS | | NO. 25 |
| | IANNGHSVQV DFNDSDDRTV VSGGPLDGPY | | |
| | RLKQFHFHWG KKHGVGSEHT VDGKSFPSEL | | |
| | HLVHWNAKKY STFGEAASAP DGLAVVGVFL | | |
| | ETGDEHPSMN RLTDALYMVR FKGTKAQFSC | | |
| | FNPKCLLPAS RHYWTYPGSL TTPPLSESVT | | |
| | WIVLREPIRI SERQMEKFRS LLFTSEEDER | | |
| | IHMVNNFRPP QPLKGRVVKA SFRA | | |
| Rattus | MTVLWWPMLR EELMSKLRTG GPSNWHKLYP | EDL87229 | SEQ. ID. |
| norvegicus | IAQGDRQSPI NIISSQAVYS PSLQPLELFY | | NO. 26 |
| | EACMSLSITN NGHSVQVDFN DSDDRTVVAG | | |
| | GPLEGPYRLK QLHFHWGKKR DVGSEHTVDG | | |
| | KSFPSELHLV HWNAKKYSTF GEAAAAPDGL | | |
| | AVVGIFLETG DEHPSMNRLT DALYMVRFKD | | |
| | TKAQFSCFNP KCLLPTSRHY WTYPGSLTTP | | |
| | PLSESVTWIV LREPIRISER QMEKFRSLLF | | |
| | TSEDDERIHM VNNFRPPQPL KGRVVKASFQ | | |
| | S | | |
| Oryctolagus | MTGHHGWGYG QDDGGRPSHW HKLYPIAQGD | XP_002711604 | SEQ. ID. |
| cuniculus | RQSPINIVSS QAVYSPGLQP LELSYEACTS | | NO. 27 |
| | LSIANNGHSV QVDFNDSDDR TVVTGGPLEG | | |
| | PYRLKQFHFH WGKRRDAGSE HTVDGKSFPS | | |
| | ELHLVHWNAR KYSTFGEAAS APDGLAVVGV | | |
| | FLETGNEHPS MNRLTDALYM VRFKGTKAQF | | |
| | SCFNPKCLLP SSRHYWTYPG SLTTPPLSES | | |
| | VTWIVLREPI SISERQMEKF RSLLFTSEDD | | |
| | ERVHMVNNFR PPQPLRGRVV KASFRA | | |
| Mus | GQDDGPSNWH KLYPIAQGDR QSPINIISSQ | AAG16230.1 | SEQ. ID. |
| musculus | AVYSPSLQPL ELFYEACMSL SITNNGHSVQ | | NO. 28 |
| | VDFNDSDDRT VVSGGPLEGP YRLKQLHFHW | | |
| | GKKRDMGSEH TVDGKSFPSE LHLVHWNAKK | | |
| | YSTFGEAAAA PDGLAVVGVF LETGDEHPSM | | |
| | NRLTDALYMV RFKDTKAQFS CFNPKCLLPT | | |
| | SRHYWTYPGS LTTPPLSESV TWIVLREPIR | | |
| | ISERQMEKFR SLLFTSEDDE RIHMVDNFRP | | |
| | PQPLKGRVVK ASFQA | | |
| Monodelphis | MTGHHGWGYG QEDGPSEWHK LYPIAQGDRQ | XP_001364411.1 | SEQ. ID. |
| domestica | SPIDIVSSQA VYDPTLKPLV LAYESCMSLS | | NO. 29 |
| | IANNGHSVMV EFDDVDDRTV VNGGPLDGPY | | |
| | RLKQFHFHWG KKHSLGSEHT VDGKSFSSEL | | |
| | HLVHWNGKKY KTFAEAAAAP DGLAVVGIFL | | |
| | ETGDEHASMN RLTDALYMVR FKGTKAQFNS | | |
| | FNPKCLLPMN LSYWTYPGSL TTPPLSESVT | | |
| | WIVLKEPITI SEKQMEKFRS LLFTAEEDEK | | |
| | VRMVNNFRPP QPLKGRVVQA SFRS | | |
| Gallus | MTGHHSWGYG QDDGPAEWHK SYPIAQGNRQ | XP_414152.1 | SEQ. ID. |
| gallus | SPIDIISAKA VYDPKLMPLV ISYESCTSLN | | NO. 30 |
| | ISNNGHSVMV EFEDIDDKTV ISGGPFESPF | | |
| | RLKQFHFHWG AKHSEGSEHT IDGKPFPCEL | | |
| | HLVHWNAKKY ATFGEAAAAP DGLAVVGVFL | | |
| | EIGKEHANMN RLTDALYMVK FKGTKAQFRS | | |
| | FNPKCLLPLS LDYWTYLGSL TTPPLNESVI | | |
| | WVVLKEPISI SEKQLEKFRM LLFTSEEDQK | | |
| | VQMVNNFRPP QPLKGRTVRA SFKA | | |
| Taeniopygia | MTGQHSWGYG QADGPSEWHK AYPIAQGNRQ | XP_002190292.1 | SEQ. ID. |
| guttata | SPIDIDSARA VYDPSLQPLL ISYESCSSLS | | NO. 31 |
| | ISNTGHSVMV EFEDTDDRTA ISGGPFQNPF | | |
| | RLKQFHFHWG TTHSQGSEHT IDGKPFPCEL | | |
| | HLVHWNARKY TTFGEAAAAP DGLAVVGVFL | | |
| | EIGKEHASMN RLTDALYMVK FKGTKAQFRG | | |
| | FNPKCLLPLS LDYWTYLGSL TTPPLNESVT | | |
| | WIVLKEPIRI SVKQLEKFRM LLFTGEEDQR | | |
| | IQMANNFRPP QPLKGRIVRA SFKA | | |

TABLE D4
Exemplary Type XIII Carbonic Anhydrases

| | | Accession | SEQ. ID. |
|---|---|---|---|
| Organism | Sequence | Number | NO |
| Human | MSRLSWGYRE HNGPIHWKEF FPIADGDQQS | NP_940986.1 | SEQ. ID. |
| | PIEIKTKEVK YDSSLRPLSI KYDPSSAKII | | NO. 32 |
| | SNSGHSFNVD FDDTENKSVL RGGPLTGSYR | | |
| | LRQVHLHWGS ADDHGSEHIV DGVSYAAELH | | |
| | VVHWNSDKYP SFVEAAHEPD GLAVLGVFLQ | | |
| | IGEPNSQLQK ITDTLDSIKE KGKQTRFTNF | | |
| | DLLSLLPPSW DYWTYPGSLT VPPLLESVTW | | |
| | IVLKQPINIS SQQLAKFRSL LCTAEGEAAA | | |
| | FLVSNHRPPQ PLKGRKVRAS FH | | |
| Pan | MSRLSWGYRE HNGPIHWKEF FPIADGDQQS | XP_001169377.1 | SEQ. ID. |
| troglodytes | PIEIKTKEVK YDSSLRPLSI KYDPSSAKII | | NO. 33 |
| | SNSGHSFNVD FDDTENKSVL RGGPLTGSYR | | |
| | LRQFHLHWGS ADDHGSEHIV DGVSYAAELH | | |
| | VVHWNSDKYP SFVEAAHEPD GLAVLGVFLQ | | |
| | IGEPNSQLQK ITDTLDSIKE KGKQTRFTNF | | |
| | DPLSLLPPSW DYWTYPGSLT VPPLLESVTW | | |
| | IVLKQPINIS SQQLAKFRSL LCTAEGEAAA | | |
| | FLVSNHRPPQ PLKGRKVRAS FH | | |
| Macaca | MSRLSWGYRE HNGPIHWKEF FPIADGDQQS | XP_001095487.1 | SEQ. ID. |
| mulatta | PIEIKTQEVK YDSSLRPLSI KYDPSSAKII | | NO. 34 |
| | SNSGHSFNVD FDDTEDKSVL RGGPLAGSYR | | |
| | LRQFHLHWGS ADDHGSEHIV DGVSYAAELH | | |
| | VVHWNSDKYP SFVEAAHEPD GLAVLGVFLQ | | |
| | IGEPNSQLQK ITDILDSIKE KGKQTRFTNF | | |
| | DPLSLLPPSW DYWTYPGSLT VPPLLESVIW | | |
| | IVLKQPINVS SQQLAKFRSL LCTAEGEAAA | | |
| | FLLSNHRPPQ PLKGRKVRAS FR | | |
| Oryctolagus | MSRISWGYGE HNGPIHWNQF FPIADGDQQS | XP_002710714.1 | SEQ. ID. |
| cuniculus | PIEIKTKEVK YDSSLRPLSI KYDPSSAKII | | NO. 35 |
| | SNSGHSFNVD FDDTEDKSVL RGGPLTGNYR | | |
| | LRQFHLHWGS ADDHGSEHVV DGVRYAAELH | | |
| | VVHWNSDKYP SFVEAAHEPD GLAVLGVFLQ | | |
| | IGEYNSQLQK ITDILDSIKE KGKQTRFTNF | | |
| | DPLSLLPSSW DYWTYPGSLT VPPLLESVTW | | |
| | IVLKQPINIS SQQLAKFRSL LCSAEGESAA | | |
| | FLLSNHRPPQ PLKGRKVRAS FH | | |
| Ailuropoda | MSRLSWGYGE HNGPIHWNKF FPIADGDQQS | XP_002916937.1 | SEQ. ID. |
| melanoleuca | PIEIKTKEVK YDSSLRPLSI KYDANSAKII | | NO. 36 |
| | SNSGHSFSVD FDDTEDKSVL RGGPLTGSYR | | |
| | LRQFHLHWGS ADDHGSEHVV DGVRYAAELH | | |
| | VVHWNSDKYP SFVEAAHEPD GLAVLGVFLQ | | |
| | IGEHNSQLQK ITDILDSIKE KGKQTRFTNF | | |
| | DPLSLLPPSW DYWTYPGSLT VPPLLESVTW | | |
| | IVLKQPINIS SEQLATFRTL LCTAEGEAAA | | |
| | FLLSNHRPPQ PLKGRKVRAS FH | | |
| Sus | MSRFSWGYGE HNGPVHWNEF FPIADGDQQS | XP_001924497.1 | SEQ. ID. |
| scrofa | PIEIKTKEVK YDSSLRPLSI KYDPSSAKII | | NO. 37 |
| | SNSGHSFSVD FDDTEDKSVL RGGPLTGSYR | | |
| | LRQFHLHWGS ADDHGSEHVV DGVKYAAELH | | |
| | VVHWNSDKYP SFVEAAHEPD GLAVLGVFLQ | | |
| | IGEHNSQLQK ITDILDSIKE KGKQTRFTNF | | |
| | DPLSLLPPSW DYWTYPGSLT VPPLLESVTW | | |
| | IILKQPINIS SQQLATFRTL LCTKEGEEAA | | |
| | FLLSNHRPLQ PLKGRKVRAS FH | | |
| Callithrix | MSRLSWGYGE HNGPIHWNEF FPIADGDRQS | XP_002759085.1 | SEQ. ID. |
| jacchus | PIEIKAKEVK YDSSLRPLSI KYDPSSAKII | | NO. 38 |
| | SNSGHSFNVD FDDTEDKSVL HGGPLTGSYR | | |
| | LRQFHLHWGS ADDHGSEHVV DGVRYAAELH | | |
| | VVHWNSEKYP SFVEAAHEPD GLAVLGVFLQ | | |
| | IGEPNSQLQK IIDILDSIKE KGKQIRFTNF | | |
| | DPLSLFPPSW DYWTYSGSLT VPPLLESVTW | | |
| | ILLKQPINIS SQQLAKFRSL LCTAEGEAAA | | |
| | FLLSNYRPPQ PLKGRKVRAS FR | | |
| Rattus | MARLSWGYDE HNGPIHWNEL FPIADGDQQS | NP_001128465.1 | SEQ. ID. |
| norvegicus | PIEIKTKEVK YDSSLRPLSI KYDPASAKII | | NO. 39 |
| | SNSGHSFNVD FDDTEDKSVL RGGPLTGSYR | | |
| | LRQFHLHWGS ADDHGSEHVV DGVRYAAELH | | |
| | VVHWNSDKYP SFVEAAHESD GLAVLGVFLQ | | |
| | IGEHNPQLQK ITDILDSIKE KGKQTRFTNF | | |
| | DPLCLLPSSW DYWTYPGSLT VPPLLESVTW | | |
| | IVLKQPISIS SQQLARFRSL LCTAEGESAA | | |
| | FLLSNHRPPQ PLKGRRVRAS FY | | |
| Mus | MARLSWGYGE HNGPIHWNEL FPIADGDQQS | NP_078771.1 | SEQ. ID. |
| musculus | PIEIKTKEVK YDSSLRPLSI KYDPASAKII | | NO. 40 |
| | SNSGHSFNVD FDDTEDKSVL RGGPLTGNYR | | |
| | LRQFHLHWGS ADDHGSEHVV DGVRYAAELH | | |
| | VVHWNSDKYP SFVEAAHESD GLAVLGVFLQ | | |
| | IGEHNPQLQK ITDILDSIKE KGKQTRFTNF | | |
| | DPLCLLPSSW DYWTYPGSLT VPPLLESVTW | | |
| | IVLKQPISIS SQQLARFRSL LCTAEGESAA | | |
| | FLLSNHRPPQ PLKGRRVRAS FY | | |
| Canis | MPPRRHGPNT FLSAGTKGQQ NFWTKNQKSG | XP_544159 | SEQ. ID. |
| familiaris | PIHWNKFFPI ADGDQQSPIE IKTKEVKYDS | | NO. 41 |
| | SLRPLSIKYD ANSAKIISNS GHSFSVDFDD | | |
| | TEDKSVLRGG PLTGSYRLRQ FHLHWGSADD | | |
| | HGSEHVVDGV RYAAELHVVH WNSDKYPSFV | | |
| | EAAHEPDGLA VLGVFLQIGE HNSQLQKITD | | |
| | ILDSIKEKGK QTRFTNFDPL SLLPPSWDYW | | |
| | TYPGSLTVPP LLESVTWIVL KQPINISSQQ | | |
| | LATFRTLLCT AEGEAAAFLL SNHRPPQPLK | | |
| | GRKVRASFH | | |
| Equus | MSGPVHWNEF FPIADGDQQS PIEIKTKEVK | XP_001489984.2 | SEQ. ID. |
| caballus | YDSSLRPLTI KYDPSSAKII SNSGHSFSVG | | NO. 42 |
| | FDDTENKSVL RGGPLTGSYR LRQFHLHWGS | | |
| | ADDHGSEHVV DGVRYAAELH IVHWNSDKYP | | |
| | SFVEAAHEPD GLAVLGVFLQ VGEHNSQLQK | | |
| | ITDTLDSIKE KGKQTLFTNF DPLSLLPPSW | | |
| | DYWTYPGSLT VPPLLESVTW IILKQPINIS | | |
| | SQQLVKFRTL LCTAEGETAA FLLSNHRPPQ | | |
| | PLKGRKVRAS FR | | |
| Bos | MSGFSWGYGE RDGPVHWNEF FPIADGDQQS | XP_002692875.1 | SEQ. ID. |
| taurus | PIEIKTKEVR YDSSLRPLGI KYDASSAKII | | NO. 43 |
| | SNSGHSFNVD FDDTDDKSVL RGGPLTGSYR | | |
| | LRQFHLHWGS TDDHGSEHVV DGVRYAAELH | | |
| | VVHWNSDKYP SFVEAAHEPD GLAVLGIFLQ | | |
| | IGEHNPQLQK ITDILDSIKE KGKQTRFTNF | | |
| | DPVCLLPPCR DYWTYPGSLT VPPLLESVTW | | |
| | IILKQPINIS SQQLAAFRTL LCSREGETAA | | |
| | FLLSNHRPPQ PLKGRKVRAS FR | | |
| Monodelphis | MSRLSWGYCE HNGPVHWSEL FPIADGDYQS | XP_001366749.1 | SEQ. ID. |
| domestica | PIEINTKEVK YDSSLRPLSI KYDPASAKII | | NO. 44 |
| | SNSGHSFSVD FDDSEDKSVL RGGPLIGTYR | | |
| | LRQFHLHWGS TDDQGSEHTV DGMKYAAELH | | |
| | VVHWNSDKYP SFVEAAHEPD GLAVLGIFLQ | | |
| | TGEHNLQMQK ITDILDSIKE KGKQIRFTNF | | |
| | DPATLLPQSW DYWTYPGSLT VPPLLESVTW | | |
| | IVLKQPITIS SQQLAKFRSL LYTGEGEAAA | | |
| | FLLSNYRPPQ PLKGRKVRAS FR | | |
| Ornithorhynchus | MKKGVGSFYE LAVNRWSVVN RVQIMIVESI | XP_001507177.1 | SEQ. ID. |
| anatinus | TEPLLCGSRA LALTLSPTQA LAVAPALALA | | NO. 45 |
| | VVQALALTVV QALALAVSPA LALSVAPALA | | |
| | LAVVQALALA VVQALALAVA QALALAVAQA | | |
| | LALAVAQALA LALPQALALT LPQALALTLS | | |
| | PTLALSVAPA LALAVAPALA LADSPALALA | | |
| | LARPHPSSGS SPALDCELVL FGDCHTVLLK | | |
| | WMRMGNYSSV SPLEERNSSC PLGPIHWNEL | | |
| | FPIADGDRQS PIEIKTKEVK YDSSLRPLSI | | |
| | KYDPTSAKII SNSGHSFSVD FDDTEDKSVL | | |
| | RGGPLSGTYR LRQFHFHWGS ADDHGSEHTV | | |
| | DGMEYSAELH VVHWNSDKYS SFVEAAHEPD | | |
| | GLAVLGIFLK RGEHNLQLQK ITDILDAIKE | | |
| | KGKQMRFTNF DPLSLLPLTR DYWTYPGSLT | | |
| | VPPLLESVIW IIFKQPISIS SQQLAKFRNL | | |
| | LYTAEGEAAD FMLSNHRPPQ PLKGRKVRAS | | |
| | FRS | | |

Human CA-II is distinguished by the fact that it is one of the fastest enzymes known in nature, with a K_{cat}/Kₘ of 1.5×10⁸ M⁻¹ S⁻¹, and accordingly in one aspect, the current invention includes the use of a human CA-II carbonic anhydrase (SEQ. ID. NO. 1).

It is well established that the genetic code is degenerate and that some amino acids have multiple codons, and accordingly, multiple polynucleotides can encode the carbonic anhydrases of the invention. Moreover, the polynucleotide sequence can be manipulated for various reasons. Examples include, but are not limited to, the incorporation of preferred codons to enhance the expression of the polynucleotide in various organisms (see generally Nakamura et al., Nuc. Acid. Res. (2000) 28 (1): 292). In addition, silent mutations can be incorporated in order to introduce, or eliminate restriction sites, remove cryptic splice sites, or manipulate the ability of single stranded sequences to form stem-loop structures: (see, e.g., Zuker M., Nucl. Acid Res. (2003); 31(13): 3406-3415). In addition, expression can be further optimized by including consensus sequences at and around the start codon.

Accordingly, and by way of example, the human nucleic acid sequence encoding human CA II. (SEQ. ID. No. 46) (below), can be codon optimized for efficient chloroplast expression in any specific photosynthetic organism of interest, as illustrated by SEQ ID No. 47 (Table D5), which represents the codon optimized DNA sequence for chloroplast expression in Chlamydomonas reinhardtii.

TABLE D5
Exemplary CA II DNA expression constructs for chloroplast expression

| | |
|---|---|
| ATGTCCCATC ACTGGGGGTA CGGCAAACAC AACGGACCTG AGCACTGGCA | SEQ. ID. NO. 46 |
| TAAGGACTTC CCCATTGCCA AGGGAGAGCG CCAGTCCCCT GTTGACATCG | (human cDNA |
| ACACTCATAC AGCCAAGTAT GACCCTTCCC TGAAGCCCCT GTCTGTTTCC | sequence) |
| TATGATCAAG CAACTTCCCT GAGGATCCTC AACAATGGTC ATGCTTTCAA | |
| CGTGGAGTTT GATGACTCTC AGGACAAAGC AGTGCTCAAG GGAGGACCCC | |
| TGGATGGCAC TTACAGATTG ATTCAGTTTC ACTTTCACTG GGGTTCACTT | |
| GATGGACAAG GTTCAGAGCA TACTGTGGAT AAAAAGAAAT ATGCTGCAGA | |
| ACTTCACTTG GTTCACTGGA ACACCAAATA TGGGGATTTT GGGAAAGCTG | |
| TGCAGCAACC TGATGGACTG GCCGTTCTAG GTATTTTTTT GAAGGTTGGC | |
| AGCGCTAAAC CGGGCCTTCA GAAAGTTGTT GATGTGCTGG ATTCCATTAA | |
| AACAAAGGGC AAGAGTGCTG ACTTCACTAA CTTCGATCCT CGTGGCCTCC | |
| TTCCTGAATC CTTGGATTAC TGGACCTACC CAGGCTCACT GACCACCCCT | |
| CCTCTTCTGG AATGTGTGAC CTGGATTGTG CTCAAGGAAC CCATCAGCGT | |
| CAGCAGCGAG CAGGTGTTGA AATTCCGTAA ACTTAACTTC AATGGGGAGG | |
| GTGAACCCGA AGAACTGATG GTGGACAACT GGCGCCCAGC TCAGCCACTG | |
| AAGAACAGGC AAATCAAAGC TTCCTTCAAA | |
| TAA | |
| gaattcATGTCtCATCAtTGGGGtTAtGGtAAACACAAtGGtCCTGAaCACTGGC | SEQ. ID. NO. 47 |
| ATAAaGACTTtCCaATTGCaAAaGGtGAaCGtCAaTCaCCTGTTGAtATtGACAC | (Optimized for |
| TCATACAGCtAAaTATGACCCTTCttTaAAaCCatTaTCTGTTTCaTATGATCAA | chloroplast |
| GCAACTTCttTacGtATttTaAACAATGGTCATGCTTTtAAtGTaGAaTTTGATG | expression) |
| ACTCTCAaGAtAAAGCAGTatTaAAaGGtGGtCCatTaGATGGtACTTACcGtTT | |
| aATTCAaTTTCACTTTCACTGGGGTTCAtTaGATGGtCAAGGTTCAGAaCATACT | |
| GTaGATAAAAAaAAATATGCTGCAGAAtTaCACTTaGTTCACTGGAACACaAAAT | |
| ATGGtGATTTTGGtAAAGCTGTaCAaCAACCTGATGGttTaGCtGTTtTAGGTAT | |
| TTTTTTaAAaGTTGGtAGtGCTAAACCaGGtCTTCAaAAAGTTGTTGATGTatTa | |
| GATTCaATTAAAACAAAaGGtAAaAGTGCTGACTTtACTAAtTTCGATCCTCGTG | |
| GttTaCTTCCTGAATCtTTaGATTACTGGACaTAtCCAGGtTCAtTaACaACaCC | |
| TCCTCTTtTaGAATGTGTaACaTGGATTGTatTaAAaGAACCaATtAGtGTaAGt | |
| AGtGAaCAaGTaTTaAAATTCCGTAAACTTAAtTTCAATGGtGAaGGTGAACCaG | |
| AAGAAtTaATGGTtGAtAACTGGCGtCCAGCTCAaCCAtTaAAaAAtcGtCAAAT | |
| tAAAGCTTCaTTCAAATAAgcatgc | |

In Table D5, the underlined sequences represent restriction sites, and bases changed to optimize chloroplast expression are listed in lower case. Table D6 provides a breakdown of the number and type of each codon optimized.

TABLE D6
Codons in Human CA II optimized for expression in
chloroplast of Chlamydomonas reinhardtii

| | | Number | | |
|---|---|---|---|---|
| | | of codons | | Expected |
| Amino | Total | that were | No. of amino | ratio of |
| acid | number | optimized | acids of each codon | codons |
| Ser(S) | 18 | 12 | TCT TCA AGT (7:7:5) | 1:1:1 |
| Phe(F) | 12 | 3 | TTT TTC (8:4) | 2:1 |
| Leu(L) | 26 | 19 | TTA CTT (21:5) | 5:1 |
| Val(V) | 17 | 10 | GTT GTA (8:9) | 1:1 |
| Pro(P) | 17 | 6 | CCT CCA (8:9) | 3:4 |
| Thr(T) | 12 | 5 | ACT ACA (5:7) | 2:3 |
| Ala(A) | 13 | 3 | GCT GCA (9:4) | 2:1 |
| Tyr(Y) | 8 | 2 | TAT TAC (6:2) | 2:1 |
| His(H) | 12 | 1 | CAT CAC (6:6) | 1:1 |
| Asn(N) | 10 | 4 | AAT AAC (7:3) | 2.5:1 |
| Asp(D) | 19 | 3 | GAT GAC (14:5) | 2.5:1 |
| Ile(I) | 9 | 4 | ATT (9) | 1 |
| Met(M) | 2 | 0 | ATG (2) | 1 |
| Gln(Q) | 11 | 7 | CAA (11) | 1 |
| Glu(E) | 13 | 6 | GAA (13) | 1 |
| Lys(K) | 24 | 11 | AAA (24) | 1 |
| Cys(C ) | 1 | 0 | TGT (1) | 1 |
| Trp(W) | 7 | 0 | TGG (7) | 1 |
| Gly(G) | 22 | 17 | GGT (22) | 1 |
| Arg( R) | 7 | 5 | CGT (7) | 1 |

Such codon optimization can be completed by standard analysis of the preferred codon usage for the host organism in question, and the synthesis of an optimized nucleic acid via standard DNA synthesis. A number of companies provide such services on a fee for services basis and include for example, DNA2.0, (CA, USA) and Operon Technologies. (CA, USA).

The carbonic anhydrase may be in its native form, i.e., as different apo forms, or allelic variants as they appear in nature, which may differ in their amino acid sequence, for example, by proteolytic processing, including by truncation (e.g., from the N- or C-terminus or both) or other amino acid deletions, additions, insertions, substitutions.

Naturally-occurring chemical modifications including post-translational modifications and degradation products of the carbonic anhydrase, are also specifically included in any of the methods of the invention including for example, pyroglutamyl, iso-aspartyl, proteolytic, phosphorylated, glycosylated, reduced, oxidatized, isomerized, and deaminated variants of the carbonic anhydrase.

The carbonic anhydrase which may be used in any of the methods and plants of the invention may have amino acid sequences which are substantially homologous, or substantially similar to any of the native CA amino acid sequences, for example, to any of the native carbonic anhydrase gene sequences listed in Tables D2-D5.

Alternatively, the carbonic anhydrase may have an amino acid sequence having at least 30% preferably at least 40, 50, 60, 70, 75, 80, 85, 90, 95, 98, or 99% identity with a CA listed in Tables D2-D5. In one aspect, the carbonic anhydrase for use in any of the methods and plants of the present invention is at least 80% identical to the mature human carbonic anhydrase (SEQ. ID. NO. 1).

```
    1 MSHHWGYGKH NGPEHWHKDF PIAKGERQSP VDIDTHTAKY DPSLKPLSVS YDQATSLRIL
  61 NNGHAFNVEF DDSQDKAVLK GGPLDGTYRL IQFHFHWGSL DGQGSEHTVD KKKYAAELHL
121 VHWNTKYGDF GKAVQQPDGL AVLGIFLKVG SAKPGLQKVV DVLDSIKTKG KSADFTNFDP
181 RGLLPESLDY WTYPGSLTTP PLLECVTWIV LKEPISVSSE QVLKFRKLNF NGEGEPEELM
241 VDNWRPAQPL KNRQIKASFK
```

It is known in the art to synthetically modify the sequences of proteins or peptides, while retaining their useful activity, and this may be achieved using techniques which are standard in the art and widely described in the literature, e.g., random or site-directed mutagenesis, cleavage, and ligation of nucleic acids, or via the chemical synthesis or modification of amino acids or polypeptide chains. For instance, conservative amino acid mutations changes can be introduced into carbonic anhydrase and are considered within the scope of the invention. Mutations of CA that modulate the stability or activity of the protein are known and may be used in the methods and plants of the invention.

The CA amino acid sequence may thus include one or more amino acid deletions, additions, insertions, and/or substitutions based on any of the naturally-occurring isoforms of the carbonic anhydrase gene. These may be contiguous or non-contiguous. Representative variants may include those having 1 to 10, or more preferably 1 to 4, 1 to 3, or 1 or 2 amino acid substitutions, insertions, and/or deletions as compared to any of sequences listed in Tables D2-D5.

The variants, derivatives, and fusion proteins of the carbonic anhydrase gene are functionally equivalent in that they have detectable carbonic anhydrase activity. More particularly, they exhibit at least 5%, at least 10%, at least 20%, at least 30%, at least 40%, preferably at least 60%, more preferably at least 80% of the activity of the human carbonic anhydrase type II gene (SEQ. ID. NO. 1), and are thus they are capable of substituting for carbonic anhydrase itself.

Such activity means any activity exhibited by a native carbonic anhydrase, whether a physiological response exhibited in an in vivo or in vitro test system, or any biological activity or reaction mediated by a native CA, e.g., in an enzyme, or cell based assay. All such variants, derivatives, fusion proteins, or fragments of the carbonic anhydrase are included, and may be used in any of the polynucleotides, vectors, host cell and methods disclosed and/or claimed herein, and are subsumed under the terms “carbonic anhydrase” or “CA”.

In other embodiments, fusion proteins of the carbonic anhydrase to other proteins are also included, and these fusion proteins may increase the biological activity, subcellular targeting, biological life, and/or ability of the CA to impact carbon dioxide utilization by RubisCO.

A fusion protein approach contemplated for use within the present invention includes the fusion of the CA to a protein-protein interaction domain, or multimerization domain to enable a direct functional association with RubisCO. Representative multimerization domains include without limitation coiled-coil dimerization domains such as leucine zipper domains which are found in certain DNA-binding polypeptides, the dimerization domain of an immunoglobulin Fab constant domain, such as an immunoglobulin heavy chain CH1 constant region or an immunoglobulin light chain constant region, the STAS domain, and other protein-protein interaction domains as provided in Tables D10 and D11. In certain embodiments, the CA intrinsincally includes a protein-protein interaction domain.

It will be appreciated that a flexible molecular linker (or spacer) optionally may be interposed between, and covalently join, the CA and any of the fusion proteins disclosed herein. Any such fusion protein may be used in any of the methods, transgenic organisms, polynucleotides and host cells of the present invention.

### III. RUBISCO

Ribulose 1,5-bisphosphate carboxylase-oxygenase activity is an enzyme activity found in plants, algae, and photosynthetic bacteria that is used in the Calvin cycle to catalyze the first major step of carbon fixation, a process by which the atoms of atmospheric CO₂ are made available to organisms in the form of energy-rich molecules (e.g. sugars). RubisCO fixes the carbon of CO₂ by carboxylating ribulose bisphosphate (“RuBP”) to form two molecules of 3-phosphoglycerate.

Three major forms of the RubisCO enzyme are found in living organisms (Andrews T. J., & Lorimer, G. H., The Biochemistry of Plants, volume 10, 131-218, 1987 and Miziorko, H. M., & Lorimer, G. H., Annu. Rev. Biochem., 52, 507-535, 1983). Form-I, which is found in higher plants, algae and most other photosynthetic organisms, is a heteromer of multiple (e.g. 8) large subunits (“ls” or “lsRubisCO”) and multiple (e.g. 8) small subunits (“ss” or “ssRubisCO”) (L, Mr=55,000) subunits, forming, for example, an LS 8 SS 8 complex. Form-II, which is primarily found in certain bacteria, e.g., the photosynthetic bacterium Rhodospirillum rubrum (R. rubrum), is a dimer of large subunits, ls2, (Tabita, F. R. and McFadden, B, A., Arch. Microbiol., 99, 231-40, 1974) that differ substantially in sequence from Form-I large subunits. Depending on the source, Form-II may be oligomerized to form dimers, tetramers, or even larger oligomers (Li, H., et al., Structure, 13, 779-789, 2005). Form-III also contains only an LS and forms dimers (ls2) or decamers ([ls2]5). In all forms, the LS subunit carries the catalytic function of the enzyme.

In higher plants, the LS subunit of the Form-I RubisCO is encoded by the chloroplast gene rbcL while the SS subunit is encoded by the nuclear gene rbcS. After synthesis, the SS subunit is translocated from the cytosol to the chloroplast, processed to remove its transit protein, and assembled with the LS subunit. The prokaryotic Form-II RubisCO (e.g., the one present in R. rubrum), has two LS subunits, encoded by a single rbcM gene (also known as cbbM). The gene for the LS subunit of R. rubrum RubisCO has been cloned and expressed in E. coli (Somerville, C. R. and Somerville, S. C., Recherche, 15, 490-501, 1984 and Pierce, J. and Gutteridge, S., Appl. Environ. Microbiol., 49, 1094-100, 1985) and shown to be a fusion protein consisting of RubisCO and 24 additional amino acids from β-galactosidase at the N-terminus. The catalytic and kinetic properties of the fusion protein were retained compared to the wild-type enzyme.

TABLE D7
Exemplary Rubisco Large Subunit gene Sequences

| | | Gene Bank | |
|---|---|---|---|
| | | Accession | SEQ. ID. |
| Organism | Sequence | Number | NO. |
| Chlamydomonas | MVPQTETKAG AGFKAGVKDY RLTYYTPDYV | NP_958405.1 | SEQ. ID. |
| reinhardtii | VRDTDILAAF RMTPQLGVPP EECGAAVAAE | | NO. 48 |
| | SSTGTWTTVW TDGLTSLDRY KGRCYDIEPV | | |
| | PGEDNQYIAY VAYPIDLFEE GSVTNMFTSI | | |
| | VGNVFGFKAL RALRLEDLRI PPAYVKTFVG | | |
| | PPHGIQVERD KLNKYGRGLL GCTIKPKLGL | | |
| | SAKNYGRAVY ECLRGGLDFT KDDENVNSQP | | |
| | FMRWRDRFLF VAEAIYKAQA ETGEVKGHYL | | |
| | NATAGTCEEM MKRAVCAKEL GVPIIMHDYL | | |
| | TGGFTANTSL AIYCRDNGLL LHIHRAMHAV | | |
| | IDRQRNHGIH FRVLAKALRM SGGDHLHSGT | | |
| | VVGKLEGERE VTLGFVDLMR DDYVEKDRSR | | |
| | GIYFTQDWCS MPGVMPVASG GIHVWHMPAL | | |
| | VEIFGDDACL QFGGGTLGHP WGNAPGAAAN | | |
| | RVALEACTQA RNEGRDLARE GGDVIRSACK | | |
| | WSPELAAACE VWKEIKFEFD TIDKL | | |
| Arabidopsis | MSPQTETKAS VGFKAGVKEY KLTYYTPEYE | AAB68400.1 | SEQ. ID. |
| thaliana | TKDTDILAAF RVTPQPGVPP EEAGAAVAAE | | NO. 49 |
| | SSTGTWTTVW TDGLTSLDRY KGRCYHIEPV | | |
| | PGEETQFIAY VAYPLDLFEE GSVTNMFTSI | | |
| | VGNVFGFKAL AALRLEDLRI PPAYTKTFQG | | |
| | PPHGIQVERD KLNKYGRPLL GCTIKPKLGL | | |
| | SAKNYGRAVY ECLRGGLDFT KDDENVNSQP | | |
| | FMRWRDRFLF CAEAIYKSQA ETGEIKGHYL | | |
| | NATAGTCEEM IKRAVFAREL GVPIVMHDYL | | |
| | TGGFTANTSL SHYCRDNGLL LHIHRAMHAV | | |
| | IDRQKNHGMH FRVLAKALRL SGGDHIHAGT | | |
| | VVGKLEGDRE STLGFVDLLR DDYVEKDRSR | | |
| | GIFFTQDWVS LPGVLPVASG GIHVWHMPAL | | |
| | TEIFGDDSVL QFGGGTLGHP WGNAPGAVAN | | |
| | RVALEACVQA RNEGRDLAVE GNEIIREACK | | |
| | WSPELAAACE VWKEITFNFP TIDKLDGQE | | |
| Capsella | MSPQTETKAS VGFKAGVKEY KLTYYTPEYE | YP_001123381.1 | SEQ. ID. |
| bursa-pastoris | TKDTDILAAF RVTPQPGVPP EEAGAAVAAE | | NO. 50 |
| | SSTGTWTTVW TDGLTSLDRY KGRCYHIEPV | | |
| | PGEETQFIAY VAYPLDLFEE GSVTNMFTSI | | |
| | VGNVFGFKAL AALRLEDLRI PPAYTKTFQG | | |
| | PPHGIQVERD KLNKYGRPLL GCTIKPKLGL | | |
| | SAKNYGRAVY ECLRGGLDFT KDDENVNSQP | | |
| | FMRWRDRFLF CAEAIYKSQA ETGEIKGHYL | | |
| | NATAGTCEEM IKRAVFAREL GVPIVMHDYL | | |
| | TGGFTANTSL SHYCRDNGLL LHIHRAMHAV | | |
| | IDRQKNHGMH FRVLAKALRL SGGDHIHAGT | | |
| | VVGKLEGDRE STLGFVDLLR DDYVEKDRSR | | |
| | GIFFTQDWVS LPGVLPVASG GIHVWHMPAL | | |
| | TEIFGDDSVL QFGGGTLGHP WGNAPGAVAN | | |
| | RVALEACVQA RNEGRDLAVE GNEIIREACK | | |
| | WSPELAAACE VWKEIRFNFP TIDKLDGQE | | |
| Crucihimalaya | MSPQTETKAS VGFKAGVKEY KLTYYTPEYE | YP_001123470.1 | SEQ. ID. |
| wallichii] | TKDTDILAAF RVTPQPGVPP EEAGAAVAAE | | NO. 51 |
| | SSTGTWTTVW TDGLTSLDRY KGRCYHIEPV | | |
| | PGEETQFIAY VAYPLDLFEE GSVTNMFTSI | | |
| | VGNVFGFKAL AALRLEDLRI PPAYTKTFQG | | |
| | PPHGIQVERD KLNKYGRPLL GCTIKPKLGL | | |
| | SAKNYGRAVY ECLRGGLDFT KDDENVNSQP | | |
| | FMRWRDRFLF CAEAIYKSQA ETGEIKGHYL | | |
| | NATAGTCEEM IKRAVFAREL GVPIVMHDYL | | |
| | TGGFTANTSL AHYCRDNGLL LHIHRAMHAV | | |
| | IDRQKNHGMH FRVLAKALRL SGGDHIHAGT | | |
| | VVGKLEGDRE STLGFVDLLR DDYVEKDRSR | | |
| | GIFFTQDWVS LPGVLPVASG GIHVWHMPAL | | |
| | TEIFGDDSVL QFGGGTLGHP WGNAPGAVAN | | |
| | RVALEACVQA RNEGRDLAVE GNEIIREACK | | |
| | WSPELAAACE VWKEIRFNFP TIDKLDGQE | | |
| Arabis hirsuta | MSPQTETKAS VGFKAGVKEY KLTYYTPEYE | YP_001123207.1 | SEQ. ID. |
| | TKDTDILAAF RVTPQPGVPP EEAGAAVAAE | | NO. 52 |
| | SSTGTWTTVW TDGLTSLDRY KGRCYHIEPV | | |
| | PGEETQFIAY VAYPLDLFEE GSVTNMFTSI | | |
| | VGNVFGFKAL AALRLEDLRI PPAYTKTFQG | | |
| | PPHGIQVERD KLNKYGRPLL GCTIKPKLGL | | |
| | SAKNYGRAVY ECLRGGLDFT KDDENVNSQP | | |
| | FMRWRDRFLF CAEAIYKSQA ETGEIKGHYL | | |
| | NATAGTCEEM IKRAVFAREL GVPIVMHDYL | | |
| | TGGFTANTSL AHYCRDNGLL LHIHRAMHAV | | |
| | IDRQKNHGMH FRVLAKALRL SGGDHVHAGT | | |
| | VVGKLEGDRE STLGFVDLLR DDYVEKDRSR | | |
| | GIFFTQDWVS LPGVLPVASG GIHVWHMPAL | | |
| | TEIFGDDSVL QFGGGTLGHP WGNAPGAVAN | | |
| | RVALEACVQA RNEGRDLAVE GNEIIREACK | | |
| | WSPELAAACE VWKEIRFNFP TVDKLDGQE | | |
| Draba nemorosa | MSPQTETKAS VGFKAGVKEY KLTYYTPEYE | YP_001123558.1 | SEQ. ID. |
| | TKDTDILAAF RVTPQPGVPP EEAGAAVAAE | | NO. 53 |
| | SSTGTWTTVW TDGLTSLDRY KGRCYHIEPV | | |
| | PGEETQFIAY VAYPLDLFEE GSVTNMFTSI | | |
| | VGNVFGFKAL AALRLEDLRI PPAYTKTFQG | | |
| | PPHGIQVERD KLNKYGRPLL GCTIKPKLGL | | |
| | SAKNYGRAVY ECLRGGLDFT KDDENVNSQP | | |
| | FMRWRDRFLF CAEAIYKSQA ETGEIKGHYL | | |
| | NATAGTCEEM IKRAVFAREL GVPIVMHDYL | | |
| | TGGFTANTSL SHYCRDNGLL LHIHRAMHAV | | |
| | IDRQKNHGMH FRVLAKALRL SGGDHIHAGT | | |
| | VVGKLEGDRE STLGFVDLLR DDYVEKDRSR | | |
| | GIFFTQDWVS LPGVLPVASG GIHVWHMPAL | | |
| | TEIFGDDSVL QFGGGTLGHP WGNAPGAVAN | | |
| | RVALEACVQA RNEGRDLAVE GNEIIREACK | | |
| | WSPELAAACE VWKEIRFNFP TIDKLDGQA | | |
| Lobularia | MSPQTETKAS VGFKAGVKEY KLTYYTPEYE | YP_001123733.1 | SEQ. ID. |
| maritima | TKDTDILAAF RVTPQPGVPP EEAGAAVAAE | | NO. 54 |
| | SSTGTWTTVW TDGLTSLDRY KGRCYHIEPV | | |
| | PGEETQFIAY VAYPLDLFEE GSVTNMFTSI | | |
| | VGNVFGFKAL AALRLEDLRI PPAYTKTFQG | | |
| | PPHGIQVERD KLNKYGRPLL GCTIKPKLGL | | |
| | SAKNYGRAVY ECLRGGLDFT KDDENVNSQP | | |
| | FMRWRDRFLF CAEAIYKSQA ETGEIKGHYL | | |
| | NATAGTCEEM IKRAVFAREL GVPIVMHDYL | | |
| | TGGFTANTSL AHYCRDNGLL LHIHRAMHAV | | |
| | IDRQKNHGMH FRVLAKALRL SGGDHIHAGT | | |
| | VVGKLEGDRE STLGFVDLLR DDYIEKDRSR | | |
| | GIFFTQDWVS LPGVLPVASG GIHVWHMPAL | | |
| | TEIFGDDSVL QFGGGTLGHP WGNAPGAVAN | | |
| | RVALEACVQA RNEGRDLAVE GNEIVREACK | | |
| | WSPELAAACE VWKEIRFNFP TIDKLDGQE | | |

TABLE D8
Exemplary RubisCO small Subunits

| | | Accession | SEQ. ID. |
|---|---|---|---|
| Organism | Sequence | Number | NO |
| Chlamydomonas | MAQALALADR FKGLKELPGL KADACGVQRM | XP_001696900.1 | SEQ. ID. |
| reinhardtii | TGDVGERVAI VAARDVRDKE TVMVIPENLA | | NO. 55 |
| | VTRVDAESHP VVGPLAAEAS ELTALTLWLL | | |
| | AERAAGAGSN YAGLLATLPE STLSPLLWSD | | |
| | AELEELMAGS PVLPEARSRK KALADTWAAL | | |
| | APKLAADPAR FPAGRRAAGA RKGVVVWDGA | | |
| | GSEMLLNDGR PNGELLLATG TLQDNNSSDF | | |
| | LSWPAGLVPA DRYYMMKSQV LESMGYSAAE | | |
| | EFPVYADRMP IQLLAYLRLS RVADPALLAK | | |
| | CTFEADVELS QMNEYEILQI LMGDCRERLA | | |
| | SYTKSYEEDV KIAQQSDLSP KERLAVKLRL | | |
| | GEKRIINATM EAVRRRLAPI RGIPTKSGQL | | |
| | ADPNSDLKEI FDTIESIPTA PLRLMQGLVS | | |
| | WARGDDDPEW YGKKKPGQGR K | | |
| Arabidopsis | MASSMLSSAA VVTSPAQATM VAPFTGLKSS | CAA32700.1 | SEQ. ID. |
| thaliana | ASFPVTRKAN NDITSITSNG GRVSCMKVWP | | NO. 56 |
| | PIGKKKFETL SYLPDLTDVE LAKEVDYLLR | | |
| | NKWIPCVEFE LEHGFVYREH GNTPGYYDGR | | |
| | YWTMWKLPLF GCTDSAQVLK EVEECKKEYP | | |
| | GAFIRIIGFD NTRQVQCISF IAYKPPSFTD | | |
| | A | | |
| Brassica | MASSMLSSAA VVTSPAQATM VAPFTGLKSS | P27985.1 | SEQ. ID. |
| napus | AAFPVTRKAN NDITSIASNG GRVSCMKVWP | | NO. 57 |
| | PVGKKKFETL SYLPDLTEVE LGKEVDYLLR | | |
| | NKWIPCVEFE LEHGFVYREH GSTPGYYDGR | | |
| | YWTMWKLPLF GCTDSAQVLK EVQECKTEYP | | |
| | NAFIRIIGFD NNRQVQCISF IAYKPPSFTG | | |
| | A | | |
| Raphanus | MASSMLSSAA VVTSQLQATM VAPFTGLKSS | P08135.1 | SEQ. ID. |
| sativus | AAFPVTRKTN TDITSIASNG GRVSCMKVWP | | NO. 58 |
| | PIGKKKFETL SYLPDLSDVE LAKEVDYLLR | | |
| | NKWIPCVEFE LEHGFVYREH GSTPGYYDGR | | |
| | YWTMWKLPLF GCTDSAQVLK EVQECKKEYP | | |
| | NALIRIIGFD NNRQVQCISF IAYKPPSFTD | | |
| | A | | |

TABLE D9
Exemplary RubisCO small Subunits (Subunits 2 and 3)

| | | | |
|---|---|---|---|
| Arabidopsis | MASSMFSSTA VVTSPAQATM VAPFTGLKSS | NP_198658.1 | SEQ. ID. |
| thaliana | ASFPVTRKAN NDITSITSNG GRVSCMKVWP | | NO. 59 |
| | PIGKKKFETL SYLPDLSDVE LAKEVDYLLR | | |
| | NKWIPCVEFE LEHGFVYREH GNTPGYYDGR | | |
| | YWTMWKLPLF GCTDSAQVLK EVEECKKEYP | | |
| | GAFIRIIGFD NTRQVQCISF IAYKPPSFTEA | | |
| Arabidopsis | MASSMLSSAA VVTSPAQATM VAPFTGLKSS | NP_198657.1 | SEQ. ID. |
| thaliana | AAFPVTRKTN KDITSIASNG GRVSCMKVWP | | NO. 60 |
| | PIGKKKFETL SYLPDLSDVE LAKEVDYLLR | | |
| | NKWIPCVEFE LEHGFVYREH GNTPGYYDGR | | |
| | YWTMWKLPLF GCTDSAQVLK EVEECKKEYP | | |
| | GAFIRIIGFD NTRQVQCISF IAYKPPSFTEA | | |
| Brassica napus | MAYSMLSSAA VVTSPAQATM VAPFTGLKSS | ABB51649.1 | SEQ. ID. |
| | AAFPVTRKAN NDITSIASNG GRVSCMKVWP | | NO. 61 |
| | PVGKKKFETL SYLPDLTEVE LGKEVDYLLR | | |
| | NKWIPCVEFE LEHGFVYREH GSTPGYYDGR | | |
| | YWTMWKLPLF GCTDSAQVLK EVQECKTEYP | | |
| | NAFIRIIGFD NNRQVQCISF IAYKPPSFTGA | | |
| Brassica rapa | MAYSMLSSAA VVTSPAQATM VAPFTGLKSS | BAJ08160.1 | SEQ. ID. |
| subsp. | SAFPVTRKAN NDITSIVSNG GRVSCMKVWP | | NO. 62 |
| chinensis | PVGKKKFETL SYLPDLTEVE LGKEVDYLLR | | |
| | NKWIPCVEFE LEHGFVYREH GSTPGYYDGR | | |
| | YWTMWKLPLF GCTDSAQVLK EVQECKTEYP | | |
| | NAFIRIIGFD NNRQVQCISF IAYKPPSFTGA | | |
| Ricinus | MASSMISSAS VSRSSPAQAT MVAPFTGLKS | XP_002521232.1 | SEQ. ID. |
| communis | AASFPVTRKA NNDITSIASN GGRVQCMQVW | | NO. 63 |
| | PPLGKKKFET LSYLPDLTDE QLAKEVDYLL | | |
| | RKGWIPCLEF ELEHGFVYRE NHRSPGYYDG | | |
| | RYWTMWKLPM FGCSDSTQVL KELDEAKKAY | | |
| | PNSFIRIIGF DNRRQVQCIS FIAYKPTTFNS | | |

The RubisCO may be in its native form, i.e., as different apo forms, or allelic variants as they appear in nature, which may differ in their amino acid sequence, for example, by proteolytic processing, including by truncation (e.g., from the N- or C-terminus or both) or other amino acid deletions, additions, insertions, substitutions.

Naturally-occurring chemical modifications including post-translational modifications and degradation products of RubisCO, are also specifically included in any of the methods of the invention including for example, pyroglutamyl, iso-aspartyl, proteolytic, phosphorylated, glycosylated, reduced, oxidatized, isomerized, and deaminated variants of the RubisCO.

The RubisCO which may be used in any of the methods and plants of the invention may have amino acid sequences which are substantially homologous, or substantially similar to any of the native RubisCO amino acid sequences, for example, to any of the native RubisCO gene sequences listed in Tables D7-D9.

Alternatively, the RubisCO may have an amino acid sequence having at least 30% preferably at least 40, 50, 60, 70, 75, 80, 85, 90, 95, 98, or 99% identity with a RUBISCO listed in Tables D7-D9.

It is known in the art to synthetically modify the sequences of proteins or peptides, while retaining their useful activity, and this may be achieved using techniques which are standard in the art and widely described in the literature, e.g., random or site-directed mutagenesis, cleavage, and ligation of nucleic acids, or via the chemical synthesis or modification of amino acids or polypeptide chains. For instance, conservative amino acid mutations changes can be introduced into RubisCO and are considered within the scope of the invention. Mutations of RubisCO that modulate the stability or activity of the protein are known and may be used in the methods and plants of the invention.

The RubisCO amino acid sequence may thus include one or more amino acid deletions, additions, insertions, and/or substitutions based on any of the naturally-occurring isoforms of the RubisCO gene. These may be contiguous or non-contiguous. Representative variants may include those having 1 to 10, or more preferably 1 to 4, 1 to 3, or 1 or 2 amino acid substitutions, insertions, and/or deletions as compared to any of sequences listed in Tables D7-D9.

The variants, derivatives, and fusion proteins of the RubisCO gene are functionally equivalent in that they have detectable RubisCO activity. More particularly, they exhibit at least 5%, at least 10%, at least 20%, at least 30%, at least 40%, preferably at least 60%, more preferably at least 80% of the activity of the Chlamydomonas Reinhardtii RubisCO large subunit and are thus they are capable of substituting for RubisCO itself.

Such activity means any activity exhibited by a native RubisCO, whether a physiological response exhibited in an in vivo or in vitro test system, or any biological activity or reaction mediated by a native RubisCO, e.g., in an enzyme, or cell based assay. All such variants, derivatives, fusion proteins, or fragments of the RubisCO are included, and may be used in any of the polynucleotides, vectors, host cell and methods disclosed and/or claimed herein, and are subsumed under the terms “RubisCO”.

In other embodiments, fusion proteins of the RubisCO to other proteins are also included, and these fusion proteins may increase the biological activity, subcellular targeting, biological life, and/or ability of the RubisCO to impact carbon dioxide utilization by RubisCO.

A fusion protein approach contemplated for use within the present invention includes the fusion of the RubisCO to a protein-protein interaction domain, or multimerization domain to enable a direct functional association with Carbonic anhydrase. Representative multimerization domains include without limitation coiled-coil dimerization domains such as leucine zipper domains which are found in certain DNA-binding polypeptides, the dimerization domain of an immunoglobulin Fab constant domain, such as an immunoglobulin heavy chain CH1 constant region or an immunoglobulin light chain constant region, the STAS domain, and other protein-protein interaction domains as provided in Tables D10 and D11. In certain embodiments, the STAS domain is encoded by SEQ. ID. NO. 84 with or without the additional N-terminal glycines encoded by SEQ. ID. NO. 84.

It will be appreciated that a flexible molecular linker (or spacer) optionally may be interposed between, and covalently join, the RubisCO and any of the fusion proteins disclosed herein. Any such fusion protein may be used in any of the methods, transgenic organisms, polynucleotides and host cells of the present invention.

As discussed above, the various forms of naturally occurring RubisCO include at least an LS subunit, while some forms also contain an SS subunit. According to the present invention, a RubisCO transformed into the photosynthetic host may be an SS subunit or an LS subunit. Optionally, the photosynthetic host is transformed with an LS subunit. Optionally, the photosynthetic host is transformed with an SS subunit. Optionally, the photosynthetic host is transformed with both an SS and an LS subunit, for example, SS and LS subunits highly homologous to each other (e.g. SS and LS subunits derived from the same genus or species). Optionally the RubisCO is xenogenic to the host. Optionally the RubisCO is derived from the host's native RubisCO.

Optionally, the donor RubisCO has either a lower or higher CO₂/O₂ selectivity than the host's native RubisCO. Optionally, the donor RubisCO has a CO₂/O₂ selectivity of greater than about 80, as is generally seen in Cyanobacteria such as Synechocystis. Optionally, the donor RubisCO enzyme has a Km of greater than in plants.

In certain embodiments, the invention provides a photosynthetic organism transformed with genes encoding both RubisCO SS and RubisCO LS derived from an organism which naturally expresses a donor RubisCO enzyme having a higher catalytic activity (Kcat) than the host's native RubisCO. Optionally, the donor RubisCO enzyme has a Kcat of greater than 3^{s-1}, for example, greater than about 5, 6, 7, or 8^{s-1}, or from about 7-20^{s-1}, or about 8-16 3^{s-1}, as is seen, for example, in red algae such as Galdieria partita.

Optionally, the donor RubisCO has a higher C_{O2}/_{O2} selectivity than the host's native RubisCO. Optionally, the donor RubisCO has a C_{O2}/_{O2} selectivity of greater than 200, for example, as is generally seen in red algae such as Galdieria partita. Optionally, the donor RubisCO has a lower km than the host's native RubisCO, for example, red algae such as Galdieria partita.

### IV. Protein-Protein Interaction Partners and Fusion Proteins Thereof.

In some embodiments, the current invention includes methods, transgenic organisms and expression vectors comprising a first fusion protein comprising a carbonic anhydrase enzyme fused in frame to a first protein-protein interaction partner; and a second fusion protein comprising a RubisCO protein subunit fused in frame to a second protein-protein interaction partner; wherein the first protein-protein interaction partner and said second protein-protein interaction partner can associate to form a protein complex.

In other embodiments, the current invention includes methods, transgenic organisms and expression vectors comprising a carbonic anhydrase enzyme, and a fusion protein comprising a RubisCO protein subunit fused in frame to a protein-protein interaction partner; wherein the protein-protein interaction partner binds to the carbonic anhydrase to form a protein complex between carbonic anhydrase and RubisCO.

In any of these methods, transgenic organisms and expression vectors, the term “protein-protein interaction partner” refers to any modular protein domain that is capable of mediating protein-protein interaction, either with its self, or a specific protein-protein interaction motif binding partner. Thus the term “protein-protein interaction pair” refers to either a single interaction domain that can bind to itself, (i.e. as a homodimer) or an appropriately selected pair of protein-protein interaction proteins (or domains) that can bind to each other to mediate the formation of a heterodimeric protein complex. Exemplary protein-protein interaction domains are listed in Table D10.

TABLE D10
Exemplary protein-protein interaction partners

| Domain | | |
|---|---|---|
| name | Exemplary Binding Partners | Consensus Binding sites |
| STAS | Carbonic anhydrase | |
| Domain | | |
| EVH1 | Class I: Ena/VASP | FPxxP (SEQ. ID. NO. 64) |
| Domain | Vinculin, Zyxin, ActA | |
| | Class II: Homer-Ves1 mGluR, IP3R, | PPxx (SEQ. ID. NO. 65) |
| | RyR | |
| WW | Yes-Associated Protein (YAP): | PPPPY (SEQ. ID. NO. 66) |
| Domain | Yes (Src-like tyrosine kinase) | |
| | Nedd4 E3 Ubiquitin Ligase: bENaC | PPPPY (SEQ. ID. NO. 66) |
| | amiloride E3 Ubiquitin Ligase sensitive | |
| | epithelial Na+ channel | |
| | FBP-11: Formin | PPLP (SEQ. ID. NO. 67) |
| SH3 Domain | Src tyrosine kinase: p85 subunit of PI | RPLPVAP (SEQ. ID. NO. 68) |
| | 3-kinase | Class I N-terminal to C-terminal |
| | | binding site |
| | Crk adaptor protein: C3G guanidine | PPPALPPKKR (SEQ. ID. NO. 69) |
| | nucleotide exchanger | Class II C-terminal to N-terminal |
| | | binding site |
| | FYB (FYN binding protein): SKAP55 | RKGDYASY (SEQ. ID. NO. 70) |
| | Adaptor protein | unconventional |
| | Pex13p (integral peroxisomal membrane | WXXQF (SEQ. ID. NO. 71) |
| | protein) Pex5p - PTS1 receptor | unconventional |
| GYF | CDBP2: CD2 | PPPPGHR (SEQ. ID. NO. 72) |
| Domain | | |

In some embodiments of the methods, transgenic organisms and expression vectors, the protein-protein interaction domain is a STAS domain which is capable of binding to carbonic anhydrase. In some embodiments, the STAS domain is selected from the proteins comprising C-terminal STAS domains listed in Table D11.

TABLE D11
Exemplary STAS protein-protein interaction domain containing proteins

| | | Accession | SEQ. ID. |
|---|---|---|---|
| Organism | Sequence | Number | NO |
| Homo sapiens | MGLADASGPRDTQALLSATQAMDLRRRDYHMERPLLNQEHLEELGR | AK297695.1 | SEQ. ID. |
| | WGSAPRTHQWRTWLQCSRARAYALLLQHLPVLVWLPRYPVRDWLLG | | NO. 73. |
| | DLLSGLSVAIMQLPQGLAYALLAGLPPVFGLYSSFYPVFIYFLFGT | | |
| | SRHISVATPGPLPLLTAPGRPTGGAGPDPLRLRGHLPVRTSCPRLY | | |
| | HSCSCAGLRLTAQVCVWPPSEQPLWATVPHLLLEVCWKLPQSKVGT | | |
| | VVTAAVAGVVLVVVKLLNDKLQQQLPMPIPGELLTLIGATGISYGM | | |
| | GLKHRFEVDVVGNIPAGLVPPVAPNTQLFSKLVGSAFTIAVVGFAI | | |
| | AISLGKIFALRHGYRVDSNQELVALGLSNLIGGIFQCFPVSCSMSR | | |
| | SLVQESIGGNSQVAGAISSLFILLIIVKLGELFHDLPKAVLAAIII | | |
| | VNLKGMLRQLSDMRSLWKANRADLLIWLVTFTATILLNLDLGLVVA | | |
| | VIFSLLLVVVRTQMPHYSVLGQVPDTDIYRDVAEYSEAKEVRGVKV | | |
| | FRSSATVYFANAEFYSDALKQRCGVDVDFLISQKKKLLKKQEQLKL | | |
| | KQLQKEEKLRKQAASPKGASVSINVNTSLEDMRSNNVEDCKMMQVS | | |
| | SGDKMEDATANGQEDSKAPDGSTLKALGLPQPDFHSLILDLGALSF | | |
| | VDTVCLKSLKNIFHDFREIEVEVYMAACHSPVVSQLEAGHFFDASI | | |
| | TKKHLFASVHDAVTFALQHPRPVPDSPVSVTRL | | |
| Homo sapiens | MGLADASGPRDTQALLSATQAMDLRRRDYHMERPLLNQEHLEELGR | NM_022911 | SEQ. ID. |
| | WGSAPRTHQWRTWLQCSRARAYALLLQHLPVLVWLPRYPVRDWLLG | | NO. 74. |
| | DLLSGLSVAIMQLPQGLAYALLAGLPPVFGLYSSFYPVFIYFLFGT | | |
| | SRHISVGTFAVMSVMVGSVTESLAPQALNDSMINETARDAARVQVA | | |
| | STLSVLVGLFQVGLGLIHFGFVVTYLSEPLVRGYTTAAAVQVFVSQ | | |
| | LKYVFGLHLSSHSGPLSLIYIVLEVCWKLPQSKVGIVVTAAVAGVV | | |
| | LVVVKLLNDKLQQQLPMPIPGELLTLIGATGISYGMGLKHRFEVDV | | |
| | VGNIPAGLVPPVAPNTQLFSKLVGSAFTIAVVGFAIAISLGKIFAL | | |
| | RHGYRVDSNQELVALGLSNLIGGIFQCFPVSCSMSRSLVQESTGGN | | |
| | SQVAGAISSLFILLIIVKLGELFHDLPKAVLAAIIIVNLKGMLRQL | | |
| | SDMRSLWKANRADLLIWLVTFTATILLNLDLGLVVAVIFSLLLVVV | | |
| | RTQMPHYSVLGQVPDTDIYRDVAEYSEAKEVRGVKVFRSSATVYFA | | |
| | NAEFYSDALKQRCGVDVDFLISQKKKLLKKQEQLKLKQLQKEEKLR | | |
| | KQAASPKGASVSINVNTSLEDMRSNNVEDCKMMQVSSGDKMEDATA | | |
| | NGQEDSKAPDGSTLKALGLPQPDFHSLILDLGALSFVDTVCLKSLK | | |
| | NIFHDFREIEVEVYMAACHSPVVSQLEAGHFFDASITKKHLFASVH | | |
| | DAVTFALQHPRPVPDSPVSVTRL | | |
| Canis | MGAGAGAPPAPEGCVRSHSSAARGLASGRGRRLSVEEPRPGGGSPW | XM_846176.1 | SEQ. ID. |
| familiaris | VDKRFTEYSTYLTGANFPVRQRDTQALLPVPQAMELRKRDYHVERP | | NO. 75. |
| | LLNQEQLEELGCWTSATGTRQWRTWFQCSRARARALLFQHLPVLAW | | |
| | LPRYPLRDWLLGDLLAGLSVAIMQLPQGLAYALLAGLPPVFGLYSS | | |
| | FYPVFVYFLFGTSRHISVGTFAVMSVMVGSVTESLAPDENFLQAVN | | |
| | STIDEATRDATRVELASTLSVLVGLFQVGLGLVRFGFVVTYLSEPL | | |
| | VRGYTTAASVQVFVSQLKYVFGLQLSSRSGPLSLIYTVLEVCSKLP | | |
| | QNVVGTVVTAVVAGVVLVLVKLLNDKLHRRLPLPIPGELLTLIGAT | | |
| | AISYGVGLKHRFGVDIVGNIPAGLVPPAAPNPQLFASLVGYAFTIA | | |
| | VVGFAIAISLGKIFALRHGYRVDSNQELVALGLSNLIGGIFQCFPV | | |
| | SCSMSRSLVQEGAGGNTQVAGAVSSLFILIIIVKLGELFRDLPKAV | | |
| | LAAAIIVNLKGMLMQFTDIPSLWKSNRMDLLIWLVTFVATILLNLD | | |
| | IGLAVAVVFSLLLVVVRTQLPHYSVLGQVTDTDIYQDVAEYSEARE | | |
| | VPGVKVFRSSATMYFANAELYSDALKQRCGIDVDHLMSQKKKRLRK | | |
| | KEQKLKRLQKTLQKQTAASEGTSVSIHVNTSVRDMESNNVEDSKAQ | | |
| | ASTGNEVEDIAAGGQEDTKASNGSTLKALGLPQPHFHSLVLDLSAL | | |
| | SFVDTVCIKSLKNIFRDFREIEVEVYLAACHTPVVTQLEAGHFFDA | | |
| | SITKQHLFASVHDAVLFALQHPKSSPANPVLMTKL | | |
| Chlamydomonas | MAALSWQGIVAVTFTALAFVVMAADWVGPDITFTVLLAFLTAFDGQ | GU181275.1 | SEQ. ID. |
| reinhardtii | IVTVAKAAAGYGNTGLLTVVFLYWVAEGITQTGGLELIMNYVLGRS | | NO. 76. |
| | RSVHWALVRSMFPVMVLSAFLNNTPCVTFMIPILISWGRRCGVPIK | | |
| | KLLIPLSYAAVLGGTCTSIGTSTNLVIVGLQDARYAKSKQVDQAKF | | |
| | QIFDIAPYGVPYALWGFVFILLAQGFLLPGNSSRYAKDLLLAVRVL | | |
| | PSSSVVKKKLKDSGLLQQNGFDVTAIYRNGQLIKISDPSIVLDGGD | | |
| | ILYVSGELDVVEFVGEEYGLALVNQEQELAAERPFGSGEEAVFSAN | | |
| | GAAPYHKLVQAKLSKTSDLIGRTVREVSWQGRFGLIPVAIQRGNGR | | |
| | EDGRLSDVVLAAGDVLLLDTTPFYDEDREDIKTNFDGKLHAVKDGA | | |
| | AKEFVIGVKVKKSAEVVGKTVSAAGLRGIPGLFVLSVDHADGTSVD | | |
| | SSDYLYKIQPDDTIWIAADVAAVGFLSKFPGLELVQQEQVDKTGTS | | |
| | ILYRHLVQAAVSHKGPLVGKTVRDVRFRTLYNAAVVAVHRENARIP | | |
| | LKVQDIVLQGGDVLLISCHTNWADEHRHDKSFVLVQPVPDSSPPKR | | |
| | SRMIIGVLLATGMVLTQIIGGLKNKEYIHLWPCAVLIAALMLLTGC | | |
| | MNADQTRKAIMWDVYLTIAAAFGVSAALEGTGVAAKFANAIISIGK | | |
| | GAGGTGAALIAIYIATALLSELLTNNAAGAIMYPIAAIAGDALKIT | | |
| | PKDTSVAIMLGASAGFVNPFSYQTNLMVYAAGNYSVREFAIVGAPF | | |
| | QVWLMIVAGFILVYRNQWHQVWIVSWICTAGIVLLPALYFLLPTRI | | |
| | QIKIDGFFERIAAVLNPKAALERRRSLRRQVSHTRTDDSGSSGSPL | | |
| | PAPKIVA | | |
| Chlamydomonas | MGFGWQGSVSIAFTALAFVVMAADWVGPDVTFTVLLAFLTAFDGQI | GU181276.1 | SEQ. ID. |
| reinhardtii | VTVAKAAAGYGNTGLLTVIFLYWVAEGITQTGGLELIMNFVLGRSR | | NO. 77 |
| | SVHWALARSMFPVMCLSAFLNNTPCVTFMIPILISWGRRCGVPIKK | | |
| | LLIPLSYASVLGGTCTSIGTSTNLVIVGLQDARYTKAKQLDQAKFQ | | |
| | IFDIAPYGVPYALWGFVFILLIQAFLLPGNSSRYAKDLLIAVRVLP | | |
| | SSSVAKKKLKDSGLLQQSGFSVSGIYRDGKYLSKPDPNWVLEPNDI | | |
| | LYAAGEFDVVEFVGEEFGLGLVNADAETSAERPFTTGEESVFTPTG | | |
| | GAPYQKLVQATIAPTSDLIGRTVREVSWQGRFGLIPVAIQRGNGRE | | |
| | DGRLNDVVLAAGDVLILDTTPFYDEEREDSKNNFAGKVRAVKDGAA | | |
| | KEFVVGVKVKKSSEVVNKTVSAAGLRGIPGLFVLSVDRADGSSVEA | | |
| | SDYLYKIQPDDTTWIATDIGAVGFLAKFPGLELVQQEQVDKTGTSI | | |
| | LYRHLVQAAVSHKGPIVGKTVRDVRFRTLYNAAVVAVHREGARVPL | | |
| | KVQDIVLQGGDVLLISCHTNWADEHRHDKSFVLLQPVPDSSPPKRS | | |
| | RMVIGVLLATGMVLTQIVGGLKSREYIHLWPAAVLTSALMLLTGCM | | |
| | NADQARKAIYWDVYLTIAAAFGVSAALEGTGVAASFANGIISIGKN | | |
| | LHSDGAALIAIYIATAMLSELLTNNAAGAIMYPIAAIAGDALKISP | | |
| | KETSVAIMLGASAGFINPFSYQCNLMVYAAGNYSVREFAIIGAPFQ | | |
| | IWLMIVAGFILCYMKEWHQVWIVSWICTAGIVLLPALYFLLPTKVQ | | |
| | LRIDAFFDRVAQTLNPKLIIERRNSIRRQASRTGSDGTGSSDSPRA | | |
| | LGVPKVITA | | |
| Chlamydomonas | MKRNTSNVDTGGVPAPLNSTPSTRLIQNGyGDSKYETERMEFPFPE | GU181277 | SEQ. ID. |
| reinhardtii | DPRYHPRDSVKGAWEKVKEDHHHRVATYNWVDWLAFFIPCVRWLRT | | NO. 78. |
| | YRRSYLLNDIVAGISVGFMVVPQGLSYANLAGLPSVYGLYGAFLPC | | |
| | IVYSLVGSSRQLAVGPVAVTSLLLGTKLKDILPEAAGISNPNIPGS | | |
| | PELDAVQEKYNRLAIQLAFLVACLYTGVGIFRLGFVTNFLSHAVIG | | |
| | GFTSGAAITIGLSQVKYILGISIPRQDRLQDQAKTYVDNMHNMKWQ | | |
| | EFIMGTTFLFLLVLFKEVGKRSKRFKWLRPIGPLTVCIIGLCAVYV | | |
| | GNVQNKGIKIIGAIKAGLPAPTVSWWFPMPEISQLFPTAIVVMLVD | | |
| | LLESTSIARALARKNKYELHANQEIVGLGLANFAGAIFNCYTTTGS | | |
| | FSRSAVNNESGAKTGLACFITAWVVGFVLIFLTPVFAHLPYCTLGA | | |
| | IIVSSIVGLLEYEQAIYLWKVNKLDWLVWMASFLGVLFISVEIGLG | | |
| | IAIGLAILIVIYESAFPNTALVGRIPGTTIWRNIKQYPNAQLAPGL | | |
| | LVFRIDAPIYFANIQWIKERLEGFASAHRVWSQEHGVPLEYVILDF | | |
| | SPVIHIDATGLHTLETIVETLAGHGTQVVLANPSQEIIALMRRGGL | | |
| | FDMIGRDYVFITVNEAVTFCSRQMAERGYAVKEDNTSSYPHFGSRR | | |
| | TPGALPAPSSQLDSSPPTSVTESISGTPAAGTYSSIGGAVPAVAGH | | |
| | TAAGNGGSHSPSAQPGVQLTTTGSQRQQ | | |
| Physcomitrella | MTRSMPLYRG EQEEMWFSHT ESIKTTPSAT TNAPLSDGIR | XP_001766939 | SEQ. ID. |
| patens | IPRFHGVRGG PDPMHRNPDL RNVAVLLSCS VQGGEVLDLG | | NO. 79 |
| subsp. patens | VVPGAKPALY CWFGFMISSL LNCVMNCLFE FDFVESAENS | | |
| | GRELRRESDK MVQLGWESYL VLATLIAGLV VMAGDWVGPD | | |
| | FVFALMVGFL TACRVITVKE STEGFSQNGV LTVVILFVVA | | |
| | EGIGQTGGME KALNLLLGKA TSPFWAITRM FIPVAITSAF | | |
| | LNNTPIVALL IPIMIAWGRR NRISPKKLLI PLSYAAVFGG | | |
| | TLTQIGTSTN FVISSLQEKR YTQLKRPGDA KFGMFDITPY | | |
| | GIVYCIGGFL FTVIASHWLL PSDETKRHSD LLLVARVPPE | | |
| | SPVANNTVRE AGLKGMERLF LVAVERQGRV THAVGPQYLL | | |
| | EPEDLLYFCG ELEQAHFYSK AFSLELLTNE AISGSKRANF | | |
| | QGEKHPSALE NGSCGSVEDS ILIMQASVRK GADIIGKTLD | | |
| | QIDFRKRFDV AVLGLKRGET HQPGPLSEMV VNANDVLVLL | | |
| | GDNEEVLQKP EVKAVFKDVE KLDEALEKEY LTGMKVTNRF | | |
| | KGVGKTVYDA GLRGINGLTL LAIDRQSGEH LKFIEDDTVV | | |
| | ELGDTLWFAG GVQGVHFLLK ISGLEHSQAP QVSKLRADIL | | |
| | YRQLVKASVA SESPLVGNTV REAHFRNKYD AVVLAIHRQG | | |
| | ERLSMDVRDV KLRAGDVLLL DTGSNFGHRY RNDAAFSLIS | | |
| | GVPESSPVKK SRMWVALFLG AAMIATQIVS SSIGGTELIN | | |
| | LFTAGILTSG LMLLTRCLSA DQARNSIDWR VYTTIAFAIA | | |
| | FSTCMEKSKL ARAIADIFIK ISESIGGMRA SYVAIYIATA | | |
| | LLSELVSNNA AAAIMYPIAA DLGDALGVVP TRMSVVVMLG | | |
| | ASAGFTLPYS YQTNLMVYAA GDYRFMEFAK FGLPCQCFMI | | |
| | ITVILIFLLD NRIWVAVGLG FALMLVVLGW HLVWEFVPAS | | |
| | IRSKFSPGRK EKTEKIEQ | | |
| stylosanthes | MSQRVSDQVM ADVIAETRSN SSSHRHGGGG GGDDTTSLPY | CAA57710.1 | SEQ. ID. |
| hamata | MHKVGTPPKQ ILFQEIKHSF NETFFPDKPF GKFKDQSGFR | | NO. 80. |
| | KLELGLQYIF PILEWGRHYD LKKFRGDFIA GLTIASLCIP | | |
| | QDLAYAKLAN LDPWYGLYSS FVAPLVYAFM GTSRDIAIGP | | |
| | VAVVSLLLGT LLSNEISNTK SHDYLRLAFT AIFFAGVTQM | | |
| | LLGVCRLGFL IDFLSHAAIV GFMAGAAIII GLQQLKGLLG | | |
| | ISNNNFTKKT DIISVMRSVW THVHHGWNWE TILIGLSFLI | | |
| | FLLITKYIAK KNKKLFWVSA ISPMISVIVS TFFVYITRAD | | |
| | KRGVSIVKHI KSGVNPSSAN EIFFHGKYLG AGVRVGVVAG | | |
| | LVALTEAIAI GRTFAAMKDY ALDGNKEMVA MGTMNIVGSL | | |
| | SSCYVTTGSF SRSAVNYMAG CKTAVSNIVM SIVVLLTLLV | | |
| | ITPLFKYTPN AVLASIIIAA VVNLVNIEAM VLLWKIDKFD | | |
| | FVACMGAFFG VIFKSVEIGL LIAVAISFAK ILLQVTRPRT | | |
| | AVLGKLPGTS VYRNIQQYPK AAQIPGMLII RVDSAIYFSN | | |
| | SNYIKERILR WLIDEGAQRT ESELPEIQHL ITEMSPVPDI | | |
| | DTSGIHAFEE LYKTLQKREV QLILANPGPV VIEKLHASKL | | |
| | TELIGEDKIF LTVADAVATY GPKTAAF | | |
| Arabidopsis | MSSRAHPVDGSPATDGGHVPMKPSPTRHKVGIPPKQNMFKDFMYTF | NM_179568 | SEQ. ID. |
| thaliana | KETFFHDDPLRDFKDQPKSKQFMLGLQSVFPVFDWGRNYTFKKFRG | | NO. 81 |
| | DLISGLTIASLCIPQDIGYAKLANLDPKYGLYSSFVPPLVYACMGS | | |
| | SRDIAIGPVAVVSLLLGTLLRAEIDPNTSPDEYLRLAFTATFFAGI | | |
| | TEAALGFFRLGFLIDFLSHAAVVGFMGGAAITIALQQLKGFLGIKK | | |
| | FTKKTDIISVLESVFKAAHHGWNWQTILIGASFLTFLLTSKIIGKK | | |
| | SKKLFWVPAIAPLISVIVSTFFVYITRADKQGVQIVKHLDQGINPS | | |
| | SFHLIYFTGDNLAKGIRIGVVAGMVALTEAVAIGRTFAAMKDYQID | | |
| | GNKEMVALGMMNVVGSMSSCYVATGSFSRSAVNFMAGCQTAVSNII | | |
| | MSIVVLLTLLFLTPLFKYTPNAILAAIIINAVIPLIDIQAAILIFK | | |
| | VDKLDFIACIGAFFGVIFVSVEIGLLIAVSISFAKILLQVTRPRTA | | |
| | VLGNIPRTSVYRNIQQYPEATMVPGVLTIRVDSAIYFSNSNYVRER | | |
| | IQRWLHEEEEKVKAASLPRIQFLIIEMSPVTDIDTSGIHALEDLYK | | |
| | SLQKRDIQLILANPGPLVIGKLHLSHFADMLGQDNIYLTVADAVEA | | |
| | CCPKLSNEV | | |

It is well established that the genetic code is degenerate and that some amino acids have multiple codons, and accordingly, multiple polynucleotides can encode the carbonic anhydrases of the invention. Moreover, the polynucleotide sequence can be manipulated for various reasons. Examples include, but are not limited to, the incorporation of preferred codons to enhance the expression of the polynucleotide in various organisms (see generally Nakamura et al., Nuc. Acid. Res. (2000) 28 (1): 292). In addition, silent mutations can be incorporated in order to introduce, or eliminate restriction sites, remove cryptic splice sites, or manipulate the ability of single stranded sequences to form stem-loop structures: (see, e.g., Zuker M., Nucl. Acid Res. (2003); 31(13): 3406-3415). In addition, expression can be further optimized by including consensus sequences at and around the start codon.

It is known in the art to synthetically modify the sequences of proteins or peptides, while retaining their useful activity, and this may be achieved using techniques which are standard in the art and widely described in the literature, e.g., random or site-directed mutagenesis, cleavage, and ligation of nucleic acids, or via the chemical synthesis or modification of amino acids or polypeptide chains. For instance, conservative amino acid mutations changes can be introduced into the protein-protein interaction domain and are considered within the scope of the invention. Mutations of the protein-protein interaction domain that modulate the stability or activity of the protein-protein interaction domains listed are known and may be used in the methods and plants of the invention.

The protein-protein interaction domain amino acid sequences may thus include one or more amino acid deletions, additions, insertions, and/or substitutions based on any of the naturally-occurring isoforms of the protein-protein interaction domains listed. These may be contiguous or non-contiguous. Representative variants may include those having 1 to 10, or more preferably 1 to 4, 1 to 3, or 1 or 2 amino acid substitutions, insertions, and/or deletions as compared to any of sequences listed in Tables D10-D11.

The variants, derivatives, and fusion proteins of the protein-protein interaction domains are functionally equivalent in that they have detectable multimerization activity. More particularly, they exhibit at least 5%, at least 10%, at least 20%, at least 30%, at least 40%, preferably at least 60%, more preferably at least 80% of the activity of the native the protein-protein interaction domains and are thus they are capable of substituting for the native domains.

A fusion protein approach contemplated for use within the present invention includes the fusion of RubisCO to a protein-protein interaction domain, or multimerization domain to enable a direct functional association with CA. Representative multimerization domains include without limitation coiled-coil dimerization domains such as leucine zipper domains which are found in certain DNA-binding polypeptides, the dimerization domain of an immunoglobulin Fab constant domain, such as an immunoglobulin heavy chain CIE constant region or an immunoglobulin light chain constant region, the STAS domain, and other protein-protein interaction domains as provided in Tables D10 and D11.

In some embodiments, the protein-protein interaction domain is a STAS domain which is fused to RubisCO that is capable of binding to CA.

It will be appreciated that a flexible molecular linker (or spacer) optionally may be interposed between, and covalently join, the RubisCO and any of the fusion proteins disclosed herein. Any such fusion protein may be used in any of the methods, transgenic organisms, polynucleotides and host cells of the present invention.

In one aspect the protein-protein interaction domain is fused to the large subunit of RubisCO. In other embodiments, the protein-protein interaction domain is fused to the small subunit of RubisCO.

An exemplary fusion protein of RubisCO to a STAS protein-protein interaction domain via a short spacer is shown below: (RUBSICO in caps, and STAS domain, and linker in small letters).

(SEQ. ID. No. 82)
ATGGTTCCACAAACAGAAACTAAAGCAGGTGCTGGATTCAAAGCCGGTGTAAAAGACTACCGTTTAACATACTAC
ACACCTGATTACGTAGTAAGAGATACTGATATTTTAGCTGCATTCCGTATGACTCCACAACTAGGTGTTCCACCT
GAAGAATGTGGTGCTGCTGTAGCTGCTGAATCTTCAACAGGTACATGGACTACAGTATGGACTGACGGTTTAACA
AGTCTTGACCGTTACAAAGGTCGTTGTTACGATATCGAACCAGTTCCGGGTGAAGACAACCAATACATTGCTTAC
GTAGCTTACCCAATCGACTTATTCGAAGAAGGTTCAGTAACTAACATGTTCACTTCTATTGTAGGTAACGTATTC
GGTTTCAAAGCTTTACGTGCTCTACGTCTTGAAGACCTTCGTATTCCACCTGCTTACGTTAAAACATTCGTAGGT
CCTCCACACGGTATTCAGGTAGAACGTGACAAATTAAACAAATATGGTCGTGGTCTTTTAGGTTGTACAATCAAA
CCTAAATTAGGTCTTTCAGCTAAAAACTACGGTCGTGCAGTTTATGAATGTTTACGTGGTGGTCTTGACTTTACT
AAAGACGACGAAAACGTAAACTCACAACCATTCATGCGTTGGCGTGACCGTTTCCTTTTCGTTGCTGAAGCTATT
TACAAAGCTCAAGCAGAAACAGGTGAAGTTAAAGGTCACTACTTAAACGCTACTGCTGGTACTTGTGAAGAAATG
ATGAAACGTGCAGTATGTGCTAAAGAATTAGGTGTACCTATTATTATGCACGACTACTTAACAGGTGGTTTCACA
GCTAACACTTCATTAGCTATCTACTGTCGTGACAACGGTCTTCTTCTACACATCCACCGTGCTATGCACGCGGTT
ATTGACCGTCAACGTAACCACGGTATTCACTTCCGTGTTCTTGCTAAAGCTCTTCGTATGTCTGGTGGTGACCAC
CTTCACTCTGGTACTGTTGTAGGTAAACTAGAAGGTGAACGTGAAGTTACTCTAGGTTTCGTAGACTTAATGCGT
GATGACTACGTTGAAAAAGACCGTAGCCGTGGTATTTACTTCACTCAAGACTGGTGTTCAATGCCAGGTGTTATG
CCAGTTGCTTCAGGCGGTATTCACGTATGGCACATGCCAGCTTTAGTTGAAATCTTCGGTGATGACGCATGTCTT
CAGTTCGGTGGTGGTACTCTAGGTCACCCTTGGGGTAACGCTCCAGGTGCTGCAGCTAACCGTGTAGCTCTTGAA
GCTTGTACTCAAGCTCGTAACGAAGGTCGTGACCTTGCTCGTGAAGGTGGCGACGTAATTCGTTCAGCTTGTAAA
TGGTCTCCAGAACTTGCTGCTGCATGTGAAGTTTGGAAAGAAATTAAATTCGAATTTGATACTATTGACAAACTT
gttgttgttgttgttgttaatcgggcggatctgcttatctggctggtgaccttcacggccaccatcttgctgaac
ctggaccttggcttggtggttgcggtcatcttctccctgctgctcgtggtggtccggacacagatgccccactac
tctgtcctggggcaggtgccagacacggatatttacagagatgtggcagagtactcagaggccaaggaagtccgg
ggggtgaaggtcttccgctcctcggccaccgtgtactttgccaatgctgagttctacagtgatgcgctgaagcag
aggtgtggtgtggatgtcgacttcctcatctcccagaagaagaaactgctcaagaagcaggagcagctgaagctg
aagcaactgcagaaagaggagaagcttcggaaacaggctgcctcccccaagggcgcctcagtttccattaatgtc
aacaccagccttgaagacatgaggagcaacaacgttgaggactgcaagatgatgcaggtgagctcaggagataag
atggaagatgcaacagccaatggtcaagaagactccaaggccccagatgggtccacactgaaggccctgggcctg
cctcagccagacttccacagcctcatcctggacctgggtgccctctcctttgtggacactgtgtgcctcaagagc
ctgaagaatattttccatgacttccgggagattgaggtggaggtgtacatggcggcctgccacagccctgtggtc
agccagcttgaggctgggcacttcttcgatgcatccatcaccaagaagcatctctttgcctctgtccatgatgct
gtcacctttgccctccaacacccgaggcctgtccccgacagccctgtttcggtcaccagactctga

### V. DNA Constructs

In one embodiment, the DNA constructs, and expression vectors of the invention include separate expression vectors each including either the carbonic anhydrase, RUBISCO fusion protein, plasma membrane bicarbonate transporter and chloroplast envelop bicarbonate transporter.

In one aspect the DNA constructs and expression vectors for carbonic anhydrase comprise polynucleotide sequences encoding any of the previously described carbonic anhydrase genes (Tables D2-D5) operatively coupled to a promoter, transit peptide sequence and transcriptional terminator for efficient expression in the photosynthetic organism of interest. In certain embodiments the CA further comprises a heterologous protein-protein interaction domain. In one aspect of any of these expression vectors, the carbonic anhydrase gene is codon optimized for expression in the photosynthetic organism of interest. In one aspect the codon optimized carbonic anhydrase gene encodes a carbonic anhydrase of SEQ. ID. NO. 1.

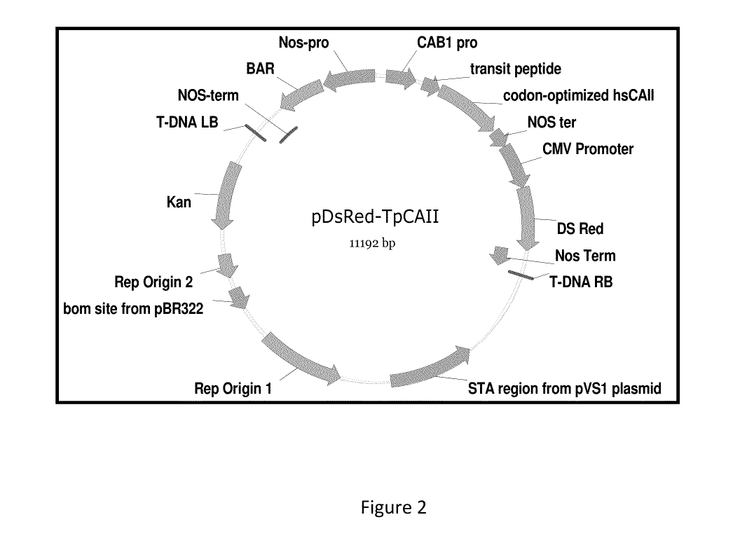

In some embodiments, the carbonic anhydrase DNA constructs and expression vectors of the invention further comprise polynucleotide sequences encoding one or more of the following elements i) a selectable marker gene to enable antibiotic selection, ii) a screenable marker gene to enable visual identification of transformed cells, and iii) T-element DNA sequences to enable Agrobacterium tumefaciens mediated transformation. An exemplary carbonic anhydrase expression cassette is shown in FIG. 2.

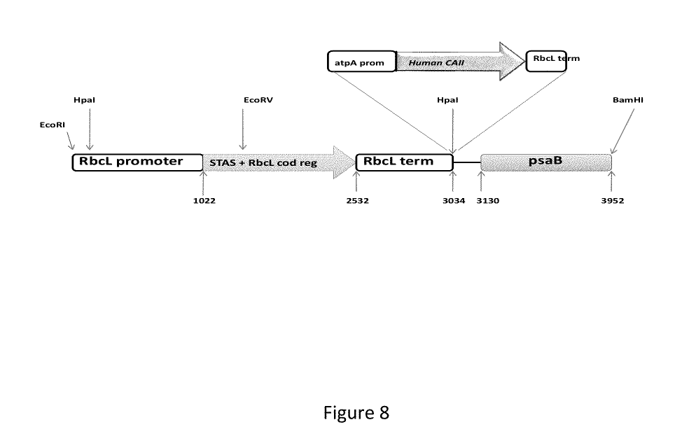

In some embodiments, the expression vectors further comprise a RubisCO-STAS fusion protein. An exemplary carbonic anhydrase expression cassette of this type is shown schematically in FIG. 8.

Those of skill in the art will appreciate that the foregoing descriptions of expression cassettes represents only illustrative examples of expression cassettes that could be readily constructed, and is not intended to represent an exhaustive list of all possible DNA constructs or expression cassettes, and combinations thereof, that could be constructed.

Moreover expression vectors suitable for use in expressing the claimed DNA constructs in plants, and methods for their construction are generally well known, and need not be limited. These techniques, including techniques for nucleic acid manipulation of genes such as subcloning a subject promoter, or nucleic acid sequences encoding a gene of interest into expression vectors, labeling probes, DNA hybridization, and the like, and are described generally in Sambrook, et al., Molecular Cloning—A Laboratory Manual (2nd Ed.), Vol. 1-3, Cold Spring Harbor Laboratory, Cold Spring Harbor, N.Y., 1989, which is incorporated herein by reference. For instance, various procedures, such as PCR, or site directed mutagenesis can be used to introduce a restriction site at the start codon of a heterologous gene of interest. Heterologous DNA sequences are then linked to a suitable expression control sequences such that the expression of the gene of interest are regulated (operatively coupled) by the promoter.

DNA constructs comprising an expression cassette for the gene of interest can then be inserted into a variety of expression vectors. Such vectors include expression vectors that are useful in the transformation of plant cells. Many other such vectors useful in the transformation of plant cells can be constructed by the use of recombinant DNA techniques well known to those of skill in the art as described above.

Exemplary expression vectors for expression in protoplasts or plant tissues include pUC 18/19 or pUC 118/119 (GIBCO BRL, Inc., MD); pBluescript SK (+/−) and pBluescript KS (+/−) (STRATAGENE, La Jolla, Calif.); pT7Blue T-vector (NOVAGEN, Inc., WI); pGEM-3Z/4Z (PROMEGA Inc., Madison, Wis.), and the like vectors, such as is described herein.

Exemplary vectors for expression using Agrobacterium tumefaciens-mediated plant transformation include for example, pBin 19 (CLONETECH), Frisch et al, Plant Mol. Biol., 27:405-409, 1995; pCAMBIA 1200 and pCAMBIA 1201 (Center for the Application of Molecular Biology to International Agriculture, Can berra, Australia); pGA482, An et al, EMBO J., 4:277-284, 1985; pCGN1547, (CALGENE Inc.) McBride et al, Plant Mol. Biol., 14:269-276, 1990, and the like vectors, such as is described herein.

Promoters.

DNA constructs will typically include promoters to drive expression of the carbonic anhydrase and bicarbonate transporters within the chloroplasts of the photosynthetic organism. Promoters may provide ubiquitous, cell type specific, constitutive promoter or inducible promoter expression. Basal promoters in plants typically comprise canonical regions associated with the initiation of transcription, such as CAAT and TATA boxes. The TATA box element is usually located approximately 20 to 35 nucleotides upstream of the initiation site of transcription. The CAAT box element is usually located approximately 40 to 200 nucleotides upstream of the start site of transcription. The location of these basal promoter elements result in the synthesis of an RNA transcript comprising nucleotides upstream of the translational ATG start site. The region of RNA upstream of the ATG is commonly referred to as a 5′ untranslated region or 5′ UTR. It is possible to use standard molecular biology techniques to make combinations of basal promoters, that is, regions comprising sequences from the CAAT box to the translational start site, with other upstream promoter elements to enhance or otherwise alter promoter activity or specificity.

In some aspects promoters may be altered to contain “enhancer DNA” to assist in elevating gene expression. As is known in the art certain DNA elements can be used to enhance the transcription of DNA. These enhancers often are found 5′ to the start of transcription in a promoter that functions in eukaryotic cells, but can often be inserted upstream (5′) or downstream (3′) to the coding sequence. In some instances, these 5′ enhancer DNA elements are introns. Among the introns that are particularly useful as enhancer DNA are the 5′ introns from the rice actin 1 gene (see U.S. Pat. No. 5,641,876), the rice actin 2 gene, the maize alcohol dehydrogenase gene, the maize heat shock protein 70 gene (U.S. Pat. No. 5,593,874), the maize shrunken 1 gene, the light sensitive 1 gene of Solanum tuberosum, and the heat shock protein 70 gene of Petunia hybrida (U.S. Pat. No. 5,659,122). For in vivo expression in plants, exemplary constitutive promoters include those derived from the CaMV 35S, rice actin, and maize ubiquitin genes, each described herein below. Exemplary inducible promoters for this purpose include the chemically inducible PR-1a promoter and a wound-inducible promoter, also described herein below. Selected promoters can direct expression in specific cell types.

Exemplary leaf specific promoters include for example, the promoter regions from the (chlorophyll a/b binding protein 1 (SI3320) (CAB1), RubisCO, photosystem I antenna protein (E01186), Xa21 protein kinase (S12429) and photosystem II oxygen-envolving complex protein (E02847). In some embodiments the promoter and associated expression control sequences can direct expression in the chloroplast, and each of these genes also includes a chloroplast targeting domain at the N-terminus. Exemplary chloroplast promoters for green algae include for example, the atpB, psbA, psbD, rbcl, and psa1 promoters, and appropriate 5′ and 3′ flanking sequences from microalgae. Other chloroplast expression systems for microalgae and plants are described in Fletcher et al., (2007) “Optimization of recombinant protein expression in the chloroplasts of green algae”. Adv. Exp. Med. Biol. 616 90-98; and Verma & Daniell (2007) “Chloroplast vector systems for biotechnology applications” Plant Physiology 145 1129-1143.

Depending upon the host cell system utilized, any one of a number of suitable promoters can be used. Promoter selection can be based on expression profile and expression level. The following are representative non-limiting examples of promoters that can be used in the expression cassettes.

35S Promoter.

The CaMV 35S promoter can be used to drive constitutive gene expression. Construction of the plasmid pCGN1761 is described in the published patent application EP 0 392 225, which a CaMV 35S promoter and the tm1 transcriptional terminator with a unique EcoRI site between the promoter and the terminator and has a pUC-type backbone.

Actin Promoter.

Several isoforms of actin are known to be expressed in most cell types and consequently the actin promoter is a good choice for a constitutive promoter. In particular, the promoter from the rice Act/gene has been cloned and characterized (McElroy et al., 1990). A 1.3 kb fragment of the promoter was found to contain inter ala the regulatory elements required for expression in rice protoplasts. Furthermore, numerous expression vectors based on the Act/promoter have been constructed specifically for use in monocotyledons are known in the art. These incorporate the Act/-intron 1, Adbl 5′ flanking sequence and Adbl-intron 1 (from the maize alcohol dehydrogenase gene) and sequence from the CaMV 35S promoter. Vectors showing highest expression were fusions of 35S and Act/intron or the Act/5′ flanking sequence and the AcV intron. Optimization of sequences around the initiating ATG (of the GUS reporter gene) also enhanced expression.

Ubiquitin Promoter.

Ubiquitin is another gene product known to accumulate in many cell types and its promoter has been cloned from several species for use in transgenic plants (e.g. sunflower, and maize). The maize ubiquitin promoter has been developed in transgenic monocot systems and its sequence and vectors constructed for monocot transformation are disclosed in the patent publication EP 0 342 926 which is herein incorporated by reference. The ubiquitin promoter is suitable for gene expression in transgenic plants, especially monocotyledons. Suitable vectors include derivatives of pAHC25, or any of the transformation vectors described in this application, modified by the introduction of the appropriate ubiquitin promoter and/or intron sequences.

Chlorophyll a/b Binding Protein 1 (CAB1) Promoter.

The CAB1 promoters from many species of plant have been cloned and may be used to direct chloroplast specific gene expression in any of the transgenic plants and methods of the invention. Exemplary CAB1 promoters include those from rice, tobacco, and wheat. (Luan & Bogorad (1992) Plant Cell. 4(8):971-81; Castresana et al., (1988) EMBO J. 7(7):1929-36; Gotor et al., (1993) Plant J. 3(4):509-18).

Inducible Expression Chemically Inducible PR-1a Promoter.

The double 35S promoter in pCGN1761ENX can be replaced with any other promoter of choice that will result in suitably high expression levels. By way of example, one of the chemically regulatable promoters described in U.S. Pat. Nos. 5,614,395 and 5,880,333 can replace the double 35S promoter. The promoter of choice is preferably excised from its source by restriction enzymes, but can alternatively be PCR-amplified using primers that carry appropriate terminal restriction sites.

The selected target gene coding sequence can be inserted into this vector, and the fusion products (i.e., promoter-gene-terminator) can subsequently be transferred to any selected transformation vector, including those described below. Various chemical regulators can be employed to induce expression of the selected coding sequence in the plants transformed according to the presently disclosed subject matter, including the benzothiadiazole, isonicotinic acid, salicylic acid and Ecdysone receptor ligands compounds disclosed in U.S. Pat. Nos. 5,523,311, 5,614,395, and 5,880,333 herein incorporated by reference.

Transcriptional Terminators

A variety of transcriptional terminators are available for use in the DNA constructs of the invention. These are responsible for the termination of transcription beyond the transgene and its correct polyadenylation.

Appropriate transcriptional terminators are those that are known to function in the relevant microalgae or plant system. Representative plant transcriptional terminators include the CaMV 35S terminator, the tm1 terminator, the nopaline synthase terminator (NOS ter), and the pea rbcS E9 terminator. With regard to RNA polymerase III terminators, these terminators typically comprise a −52 run of 5 or more consecutive thymidine residues. In one embodiment, an RNA polymerase III terminator comprises the sequence TTTTTTT. These can be used in both monocotyledons and dicotyledons.

For algal use, endogenous 5′ and 3′ elements from the genes listed above, i.e. appropriate 5′ and 3′ flanking sequences from the atpB, psbA, psbD, rbcl, actin, psaD, B-tubulin, CAB, rbcs and psa1 genes may be used.

Transit Peptide Sequences

Sequences that are joined to the coding sequence of an expressed gene, which are removed post-translationally from the initial translation product and which facilitate the transport of the protein into or through intracellular or extracellular membranes, are termed transit sequences (usually into vacuoles, vesicles, plastids and other intracellular organelles). By comparison signal sequences typically facilitate the transport of the protein into the endoplasmic reticulum, golgi apparatus, peroxisomes or glyoxysomes, and outside of the cellular membrane. By facilitating the transport of the protein into compartments inside and outside the cell, these sequences may also increase the accumulation of a gene product protecting the protein from intracellular proteolytic degradation. Various transit peptides which function as described herein are well known in the art, and are described in, for example, Johnson et al. The Plant Cell (1990) 2:525-532; Sauer et al. EMBO J. (1990) 9:3045-3050; Mueckler et al. Science (1985) 229:941-945; Von Heijne, Eur. J. Biochem. (1983) 133:17-21; Yon Heijne, J. Mol. Biol. (1986) 189:239-242; Iturriaga et al. The Plant Cell (1989) 1:381-390; McKnight et al., Nucl. Acid Res. (1990) 18:4939-4943; Matsuoka and Nakamura, Proc. Natl. Acad. Sci. USA (1991) 88:834-838. Exemplary transit signals typically comprise the motif VR↓AAAVXX (SEQ. ID. No. 83) where the downward arrow denotes the site of cleavage and “X” denotes any amino acid. (Emanuelsson et al., (1999) Prot. Sci. 8 978-984). Examples of useful transit proteins include those from ssRubisCO, the Calvin cycle enzymes and the Light harvesting complex-II gene family.

These sequences can also allow for additional mRNA sequences from highly expressed genes to be attached to the coding sequence of the genes. Since mRNA being translated by ribosomes is more stable than naked mRNA, the presence of translatable mRNA 5′ of the gene of interest may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting sequences of certain proteins may be desirable in order to enhance the stability of the protein (U.S. Pat. No. 5,545,818, incorporated herein by reference in its entirety).

Sequences for the Enhancement or Regulation of Expression

Numerous sequences have been found to enhance the expression of an operatively linked nucleic acid sequence, and these sequences can be used in conjunction with the nucleic acids of the presently disclosed subject matter to increase their expression in transgenic plants.

Various intron sequences have been shown to enhance expression, particularly in monocotyledonous cells. For example, the introns of the maize Adbl gene have been found to significantly enhance the expression of the wild-type gene under its cognate promoter when introduced into maize cells. Intron 1 was found to be particularly effective and enhanced expression in fusion constructs with the chloramphenicol acetyltransferase gene. In the same experimental system, the intron from the maize bronzes gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMY) have been shown to be effective in enhancing expression.

Selectable Markers:

For certain target species, different antibiotic or herbicide selection markers can be included in the DNA constructs of the invention. Selection markers used routinely in transformation include the npt II gene (Kan), which confers resistance to kanamycin and related antibiotics, the bar gene, which confers resistance to the herbicide phosphinothricin, the hph gene, which confers resistance to the antibiotic hygromycin, the dhfr gene, which confers resistance to methotrexate, and the EPSP synthase gene, which confers resistance to glyphosate (U.S. Pat. Nos. 4,940,935 and 5,188,642).

Screenable Markers

Screenable markers may also be employed in the DNA constructs of the present invention, including for example the β-glucuronidase or uidA gene (the protein product is commonly referred to as GUS), isolated from E. coli, which encodes an enzyme for which various chromogenic substrates are known; an R-locus gene, which encodes a product that regulates the production of anthocyanin pigments (red color) in plant tissues; a β-lactamase gene, which encodes an enzyme for which various chromogenic substrates are known (e.g., PADAC, a chromogenic cephalosporin); a xylE gene, which encodes a catechol dioxygenase that can convert chromogenic catechols; an α-amylase gene; a tyrosinase gene which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene, which allows for bioluminescence detection; an aequorin gene, which may be employed in calcium-sensitive bioluminescence detection; or a gene encoding for green fluorescent protein (PCT Publication WO 97/41228).

The R gene complex in maize encodes a protein that acts to regulate the production of anthocyanin pigments in most seed and plant tissue. Maize strains can have one, or as many as four, R alleles which combine to regulate pigmentation in a developmental and tissue specific manner. Thus, an R gene introduced into such cells will cause the expression of a red pigment and, if stably incorporated, can be visually scored as a red sector. If a maize line carries dominant alleles for genes encoding for the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 which contains the rg-Stadler allele and TR112, a K55 derivative which has the genotype r-g, b, Pl. Alternatively, any genotype of maize can be utilized if the C1 and R alleles are introduced together.

In some aspects, screenable markers provide for visible light emission or fluorescence as a screenable phenotype. Suitable screenable markers contemplated for use in the present invention include firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for population screening for bioluminescence, such as on tissue culture plates, or even for whole plant screening.

Many naturally fluorescent proteins including red and green fluorescent proteins and mutants thereof, from jelly fish and coral are commercially available (for example from CLONTECH, Palo Alto, Calif.) and provide convenient visual identification of plant transformation.

### VI. Methods of Transformation

Techniques for transforming a wide variety of plant species are well known and described in the technical and scientific literature. See, for example, Weising et al, (1988) Ann. Rev. Genet., 22:421-477. As described herein, the DNA constructs of the present invention typically contain a marker gene which confers a selectable phenotype on the plant cells. For example, the marker may encode biocide resistance, particularly antibiotic resistance, such as resistance to kanamycin, G418, bleomycin, hygromycin, or herbicide resistance, such as resistance to chlorsulfuron or Basta. Such selective marker genes are useful in protocols for the production of transgenic plants.

DNA constructs can be introduced into the genome of the desired plant host by a variety of conventional techniques. For example, the DNA construct may be introduced directly into the DNA of the plant cell using techniques such as electroporation and microinjection of plant cell protoplasts. Alternatively, the DNA constructs can be introduced directly to plant tissue using biolistic methods, such as DNA micro-particle bombardment. In addition, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al, (1984) EMBO J., 3:2717-2722. Electroporation techniques are described in Fromm et al, (1985) Proc. Natl. Acad. Sci. USA, 82:5824. Biolistic transformation techniques are described in Klein et al, (1987) Nature 327:70-7. The full disclosures of all references cited are incorporated herein by reference.

A variation involves high velocity biolistic penetration by small particles with the nucleic acid either within the matrix of small beads or particles, or on the surface (Klein et al, (1987) Nature, 327:70-73,). Although typically only a single introduction of a new nucleic acid segment is required, this method particularly provides for multiple introductions.

Agrobacterium tumefaciens-meditated transformation techniques are well described in the scientific literature. See, for example Horsch et al, (1984) Science, 233:496-498, and Fraley et al, (1983) Proc. Natl. Acad. Sci. USA, 90:4803.

More specifically, a plant cell, an explant, a meristem or a seed is infected with Agrobacterium tumefaciens transformed with the segment. Under appropriate conditions known in the art, the transformed plant cells are grown to form shoots, roots, and develop further into plants. The nucleic acid segments can be introduced into appropriate plant cells, for example, by means of the Ti plasmid of Agrobacterium tumefaciens. The Ti plasmid is transmitted to plant cells upon infection by Agrobacterium tumefaciens, and is stably integrated into the plant genome (Horsch et al, (1984) Science, 233:496-498; Fraley et al, (1983) Proc. Nat'l. Acad. Sci. U.S.A., 80:4803.

Ti plasmids contain two regions essential for the production of transformed cells. One of these, named transfer DNA (T DNA), induces tumor formation. The other, termed virulent region, is essential for the introduction of the T DNA into plants. The transfer DNA region, which transfers to the plant genome, can be increased in size by the insertion of the foreign nucleic acid sequence without its transferring ability being affected. By removing the tumor-causing genes so that they no longer interfere, the modified Ti plasmid can then be used as a vector for the transfer of the gene constructs of the invention into an appropriate plant cell, such being a “disabled Ti vector”.

All plant cells which can be transformed by Agrobacterium and whole plants regenerated from the transformed cells can also be transformed according to the invention so as to produce transformed whole plants which contain the transferred foreign nucleic acid sequence. There are various ways to transform plant cells with Agrobacterium, including: (1) co-cultivation of Agrobacterium with cultured isolated protoplasts, (2) co-cultivation of cells or tissues with Agrobacterium, or (3) transformation of seeds, apices or meristems with Agrobacterium.

Method (1) requires an established culture system that allows culturing protoplasts and plant regeneration from cultured protoplasts. Method (2) requires (a) that the plant cells or tissues can be transformed by Agrobacterium and (b) that the transformed cells or tissues can be induced to regenerate into whole plants. Method (3) requires micropropagation.

In the binary system, to have infection, two plasmids are needed: a T-DNA containing plasmid and a vir plasmid. Any one of a number of T-DNA containing plasmids can be used, the only requirement is that one be able to select independently for each of the two plasmids. After transformation of the plant cell or plant, those plant cells or plants transformed by the Ti plasmid so that the desired DNA segment is integrated can be selected by an appropriate phenotypic marker. These phenotypic markers include, but are not limited to, antibiotic resistance, herbicide resistance or visual observation. Other phenotypic markers are known in the art and may be used in this invention.

The present invention embraces use of the claimed DNA constructs in transformation of any plant, including both dicots and monocots. Transformation of dicots is described in references above. Transformation of monocots is known using various techniques including electroporation (e.g., Shimamoto et al, (1992) Nature, 338:274-276; ballistics (e.g., European Patent Application 270,356); and Agrobacterium (e.g., Bytebier et al, (1987) Proc. Nat'l Acad. Sci. USA, 84:5345-5349).

Transformed plant cells which are derived by any of the above transformation techniques can be cultured to regenerate a whole plant which possesses the desired transformed phenotype. Such regeneration techniques rely on manipulation of certain phytohormones in a tissue culture growth medium typically relying on a biocide and/or herbicide marker which has been introduced together with the nucleotide sequences. Plant regeneration from cultured protoplasts is described in Evans et al, Handbook of Plant Cell Culture, pp. 124-176, MacMillan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally by Klee et al, Ann Rev. Plant Phys., 38:467-486, 1987. Additional methods for producing a transgenic plant useful in the present invention are described in U.S. Pat. Nos. 5,188,642; 5,202,422; 5,384,253; 5,463,175; and 5,639,947. The methods, compositions, and expression vectors of the invention have use over a broad range of types of plants, and eukaryotic algae including the creation of transgenic photosynthetic organisms belonging to virtually any species. In some embodiments, the photosynthetic organism is selected from soybean, rice, wheat, oats, potato, cassaya, barley, beans, jatropha, vegetables, fruit trees, and eukaryotic alga.

Selection

Typically DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin, G418 and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA. Using the techniques disclosed herein, greater than 40% of bombarded embryos may yield transformants.

One example of a herbicide which is useful for selection of transformed cell lines in the practice of the invention is the broad spectrum herbicide glyphosate. Glyphosate inhibits the action of the enzyme EPSPS, which is active in the aromatic amino acid biosynthetic pathway Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, PCT Publication WO 97/04103. The best characterized mutant EPSPS gene conferring glyphosate resistance comprises amino acid changes at residues 102 and 106, although it is anticipated that other mutations will also be useful (PCT Publication WO 97/04103). Furthermore, a naturally occurring glyphosate resistant EPSPS may be used, e.g., the CP4 gene isolated from Agrobacterium encodes a glyphosate resistant EPSPS (U.S. Pat. No. 5,627,061).

To use the bar-bialaphos or the EPSPS-glyphosate selective systems, tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is believed that ranges of 0.1-50 mg/l bialaphos or 0.1-50 mM glyphosate will find utility in the practice of the invention. Bialaphos and glyphosate are provided as examples of agents suitable for selection of transformants, but the technique of this invention is not limited to them.

Another herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism. Synthetic PPT, the active ingredient in the herbicide LIBERTY™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity. The bar gene has been cloned and expressed in transgenic tobacco, tomato, potato, Brassica and maize (U.S. Pat. No. 5,550,318). In previous reports, some transgenic plants which expressed the resistance gene were completely resistant to commercial formulations of PPT and bialaphos in greenhouses.

It further is contemplated that the herbicide dalapon, 2,2-dichloropropionic acid, may be useful for identification of transformed cells. The enzyme 2,2-dichloropropionic acid dehalogenase (deh) inactivates the herbicidal activity of 2,2-dichloropropionic acid and therefore confers herbicidal resistance on cells or plants expressing a gene encoding the dehalogenase enzyme (U.S. Pat. No. 5,780,708).

Alternatively, a gene encoding anthranilate synthase, which confers resistance to certain amino acid analogs, e.g., 5-methyltryptophan or 6-methyl anthranilate, may be useful as a selectable marker gene. The use of an anthranilate synthase gene as a selectable marker was described in U.S. Pat. No. 5,508,468 and U.S. Pat. No. 6,118,047.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. In a similar fashion, the introduction of the C1 and B genes will result in pigmented cells and/or tissues.

The enzyme luciferase may be used as a screenable marker in the context of the present invention. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells that are expressing luciferase and manipulate cells expressing in real time. Another screenable marker which may be used in a similar fashion is the gene coding for green fluorescent protein (GFP) or a gene coding for other fluorescing proteins such as DSRED® (Clontech, Palo Alto, Calif.).

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase or GFP would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene (WO 99/60129).

Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. Preferred growth regulators for plant regeneration include cytokines such as 6-benzylamino pelerine, peahen or the like, and abscise acid. Media improvement in these and like ways has been found to facilitate the growth of cells at specific developmental stages. Tissue may be maintained on a basic media with axing type growth regulators until sufficient tissue is available to begin plant regeneration efforts, or following repeated rounds of manual selection, until the morphology of the tissue is suitable for regeneration, then transferred to media conducive to maturation of embroils. Cultures are transferred every 1-4 weeks, preferably every 2-3 weeks on this medium. Shoot development will signal the time to transfer to medium lacking growth regulators.

The transformed cells, identified by selection or screening and cultured in an appropriate medium that supports regeneration, will then be allowed to mature into plants. Developing plantlets were transferred to soilless plant growth mix, and hardened off, e.g., in an environmentally controlled chamber at about 85% relative humidity, 600 ppm CO₂, and 25-250 microeinsteins m⁻² s⁻¹ of light, prior to transfer to a greenhouse or growth chamber for maturation. Plants are preferably matured either in a growth chamber or greenhouse. Plants are regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants are preferably grown at about 19 to 28° C. After the regenerating plants have reached the stage of shoot and root development, they may be transferred to a greenhouse for further growth and testing. Plants may be pollinated using conventional plant breeding methods known to those of skill in the art and seed produced.

Progeny may be recovered from transformed plants and tested for expression of the exogenous expressible gene. Note however, that seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

Characterization

To confirm the presence of the exogenous DNA or “transgene(s)” in the regenerating plants, a variety of assays, known in the art may be performed. Such assays include, for example, “molecular biological” assays, such as Southern and Northern blotting and PCR; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

DNA Integration, RNA Expression and Inheritance

Genomic DNA may be isolated from callus cell lines or any plant parts to determine the presence of the exogenous gene through the use of techniques well known to those skilled in the art. Note, that intact sequences will not always be present, presumably due to rearrangement or deletion of sequences in the cell.

The presence of DNA elements introduced through the methods of this invention may be determined by polymerase chain reaction (PCR). Using this technique, discreet fragments of DNA are amplified and detected by gel electrophoresis. This type of analysis permits one to determine whether a gene is present in a stable transformant, but does not necessarily prove integration of the introduced gene into the host cell genome. Typically, DNA has been integrated into the genome of all transformants that demonstrate the presence of the gene through PCR analysis. In addition, it is not possible using PCR techniques to determine whether transformants have exogenous genes introduced into different sites in the genome, i.e., whether transformants are of independent origin. Using PCR techniques it is possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition, it is possible through Southern hybridization to demonstrate the presence of introduced genes in high molecular weight DNA, i.e., confirm that the introduced gene has been integrated into the host cell genome. The technique of Southern hybridization provides information that is obtained using PCR, e.g., the presence of a gene, but also demonstrates integration into the genome and characterizes each individual transformant.

It is contemplated that using the techniques of dot or slot blot hybridization, which are modifications of Southern hybridization techniques, one could obtain the same information that is derived from PCR, e.g., the presence of a gene.

Both PCR and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR techniques, referred to as RT-PCR, also may be used for detection and quantification of RNA produced from introduced genes. In this application of PCR it is first necessary to reverse transcribe RNA into DNA, using enzymes such as reverse transcriptase, and then through the use of conventional PCR techniques amplify the DNA. In most instances PC techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

It is further contemplated that TAQMAN® technology (Applied Biosystems, Foster City, Calif.) may be used to quantitate both DNA and RNA in a transgenic cell.

Gene Expression

While Southern blotting and PCR may be used to detect the gene(s) in question, they do not provide information as to whether the gene is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following an increase in fluorescence as anthranilate is produced, to name two.

Very frequently the expression of a gene product is determined by evaluating the phenotypic results of its expression. These assays also may take many forms, including but not limited to, analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

Event Specific Transgene Assay

Southern blotting, PCR and RT-PCR techniques can be used to identify the presence or absence of a given transgene but, depending upon experimental design, may not specifically and uniquely identify identical or related transgene constructs located at different insertion points within the recipient genome. To more precisely characterize the presence of transgenic material in a transformed plant, one skilled in the art could identify the point of insertion of the transgene and, using the sequence of the recipient genome flanking the transgene, develop an assay that specifically and uniquely identifies a particular insertion event. Many methods can be used to determine the point of insertion such as, but not limited to, Genome Walker™ technology (CLONTECH, Palo Alto, Calif.), Vectorette™ technology (Sigma, St. Louis, Mo.), restriction site oligonucleotide PCR, uneven PCR (Chen and Wu, 1997) and generation of genomic DNA clones containing the transgene of interest in a vector such as, but not limited to, lambda phage.

Once the sequence of the genomic DNA directly adjacent to the transgenic insert on either or both sides has been determined, one skilled in the art can develop an assay to specifically and uniquely identify the insertion event. For example, two oligonucleotide primers can be designed, one wholly contained within the transgene and one wholly contained within the flanking sequence, which can be used together with the PCR technique to generate a PCR product unique to the inserted transgene. In one embodiment, the two oligonucleotide primers for use in PCR could be designed such that one primer is complementary to sequences in both the transgene and adjacent flanking sequence such that the primer spans the junction of the insertion site while the second primer could be homologous to sequences contained wholly within the transgene. In another embodiment, the two oligonucleotide primers for use in PCR could be designed such that one primer is complementary to sequences in both the transgene and adjacent flanking sequence such that the primer spans the junction of the insertion site while the second primer could be homologous to sequences contained wholly within the genomic sequence adjacent to the insertion site. Confirmation of the PCR reaction may be monitored by, but not limited to, size analysis on gel electrophoresis, sequence analysis, hybridization of the PCR product to a specific radiolabeled DNA or RNA probe or to a molecular beacon, or use of the primers in conjugation with a TAQMAN™ probe and technology (Applied Biosystems, Foster City, Calif.).

Site Specific Integration or Excision of Transgenes

It is specifically contemplated by the inventors that one could employ techniques for the site-specific integration or excision of transformation constructs prepared in accordance with the instant invention. An advantage of site-specific integration or excision is that it can be used to overcome problems associated with conventional transformation techniques, in which transformation constructs typically randomly integrate into a host genome and multiple copies of a construct may integrate. This random insertion of introduced DNA into the genome of host cells can be detrimental to the cell if the foreign DNA inserts into an essential gene. In addition, the expression of a transgene may be influenced by “position effects” caused by the surrounding genomic DNA. Further, because of difficulties associated with plants possessing multiple transgene copies, including gene silencing, recombination and unpredictable inheritance, it is typically desirable to control the copy number of the inserted DNA, often only desiring the insertion of a single copy of the DNA sequence.

Site-specific integration can be achieved in plants by means of homologous recombination (see, for example, U.S. Pat. No. 5,527,695, specifically incorporated herein by reference in its entirety). Homologous recombination is a reaction between any pair of DNA sequences having a similar sequence of nucleotides, where the two sequences interact (recombine) to form a new recombinant DNA species. The frequency of homologous recombination increases as the length of the shared nucleotide DNA sequences increases, and is higher with linearized plasmid molecules than with circularized plasmid molecules. Homologous recombination can occur between two DNA sequences that are less than identical, but the recombination frequency declines as the divergence between the two sequences increases.

Introduced DNA sequences can be targeted via homologous recombination by linking a DNA molecule of interest to sequences sharing homology with endogenous sequences of the host cell. Once the DNA enters the cell, the two homologous sequences can interact to insert the introduced DNA at the site where the homologous genomic DNA sequences were located. Therefore, the choice of homologous sequences contained on the introduced DNA will determine the site where the introduced DNA is integrated via homologous recombination. For example, if the DNA sequence of interest is linked to DNA sequences sharing homology to a single copy gene of a host plant cell, the DNA sequence of interest will be inserted via homologous recombination at only that single specific site. However, if the DNA sequence of interest is linked to DNA sequences sharing homology to a multicopy gene of the host eukaryotic cell, then the DNA sequence of interest can be inserted via homologous recombination at each of the specific sites where a copy of the gene is located.

DNA can be inserted into the host genome by a homologous recombination reaction involving either a single reciprocal recombination (resulting in the insertion of the entire length of the introduced DNA) or through a double reciprocal recombination (resulting in the insertion of only the DNA located between the two recombination events). For example, if one wishes to insert a foreign gene into the genomic site where a selected gene is located, the introduced DNA should contain sequences homologous to the selected gene. A single homologous recombination event would then result in the entire introduced DNA sequence being inserted into the selected gene. Alternatively, a double recombination event can be achieved by flanking each end of the DNA sequence of interest (the sequence intended to be inserted into the genome) with DNA sequences homologous to the selected gene. A homologous recombination event involving each of the homologous flanking regions will result in the insertion of the foreign DNA. Thus only those DNA sequences located between the two regions sharing genomic homology become integrated into the genome.

Although introduced sequences can be targeted for insertion into a specific genomic site via homologous recombination, in higher eukaryotes homologous recombination is a relatively rare event compared to random insertion events. Thus random integration of transgenes is more common in plants. To maintain control over the copy number and the location of the inserted DNA, randomly inserted DNA sequences can be removed. One manner of removing these random insertions is to utilize a site-specific recombinase system (U.S. Pat. No. 5,527,695).

A number of different site specific recombinase systems could be employed in accordance with the instant invention, including, but not limited to, the Cre/lox system of bacteriophage P1 (U.S. Pat. No. 5,658,772, specifically incorporated herein by reference in its entirety), the FLP/FRT system of yeast, the Gin recombinase of phage Mu, the Pin recombinase of E. coli, and the R/RS system of the pSRi plasmid. The bacteriophage P1 Cre/lox and the yeast FLP/FRT systems constitute two particularly useful systems for site specific integration or excision of transgenes. In these systems, a recombinase (Cre or FLP) will interact specifically with its respective site-specific recombination sequence (lox or FRT, respectively) to invert or excise the intervening sequences. The sequence for each of these two systems is relatively short (34 bp for 10× and 47 bp for FRT) and therefore, convenient for use with transformation vectors.

The FLP/FRT recombinase system has been demonstrated to function efficiently in plant cells. Experiments on the performance of the FLP/FRT system in both maize and rice protoplasts indicate that FRT site structure, and amount of the FLP protein present, affects excision activity. In general, short incomplete FRT sites leads to higher accumulation of excision products than the complete full-length FRT sites. The systems can catalyze both intra- and intermolecular reactions in maize protoplasts, indicating its utility for DNA excision as well as integration reactions. The recombination reaction is reversible and this reversibility can compromise the efficiency of the reaction in each direction. Altering the structure of the site-specific recombination sequences is one approach to remedying this situation. The site-specific recombination sequence can be mutated in a manner that the product of the recombination reaction is no longer recognized as a substrate for the reverse reaction, thereby stabilizing the integration or excision event.

In the Cre-lox system, discovered in bacteriophage P1, recombination between lox sites occurs in the presence of the Cre recombinase (see, e.g., U.S. Pat. No. 5,658,772, specifically incorporated herein by reference in its entirety). This system has been utilized to excise a gene located between two lox sites which had been introduced into a yeast genome (Sauer, 1987). Cre was expressed from an inducible yeast GAL1 promoter and this Cre gene was located on an autonomously replicating yeast vector.

Since the lox site is an asymmetrical nucleotide sequence, lox sites on the same DNA molecule can have the same or opposite orientation with respect to each other. Recombination between lox sites in the same orientation results in a deletion of the DNA segment located between the two lox sites and a connection between the resulting ends of the original DNA molecule. The deleted DNA segment forms a circular molecule of DNA. The original DNA molecule and the resulting circular molecule each contain a single lox site. Recombination between lox sites in opposite orientations on the same DNA molecule result in an inversion of the nucleotide sequence of the DNA segment located between the two lox sites. In addition, reciprocal exchange of DNA segments proximate to lox sites located on two different DNA molecules can occur. All of these recombination events are catalyzed by the product of the Cre coding region.

Deletion of Sequences Located within the Transgenic Insert

During the transformation process it is often necessary to include ancillary sequences, such as selectable marker or reporter genes, for tracking the presence or absence of a desired trait gene transformed into the plant on the DNA construct. Such ancillary sequences often do not contribute to the desired trait or characteristic conferred by the phenotypic trait gene. Homologous recombination is a method by which introduced sequences may be selectively deleted in transgenic plants.

It is known that homologous recombination results in genetic rearrangements of transgenes in plants. Repeated DNA sequences have been shown to lead to deletion of a flanked sequence in various dicot species, e.g. Arabidopsis thaliana and Nicotiana tabacum. One of the most widely held models for homologous recombination is the double-strand break repair (DSBR) model.

Deletion of sequences by homologous recombination relies upon directly repeated DNA sequences positioned about the region to be excised in which the repeated DNA sequences direct excision utilizing native cellular recombination mechanisms. The first fertile transgenic plants are crossed to produce either hybrid or inbred progeny plants, and from those progeny plants, one or more second fertile transgenic plants are selected which contain a second DNA sequence that has been altered by recombination, preferably resulting in the deletion of the ancillary sequence. The first fertile plant can be either hemizygous or homozygous for the DNA sequence containing the directly repeated DNA which will drive the recombination event.

The directly repeated sequences are located 5′ and 3′ to the target sequence in the transgene. As a result of the recombination event, the transgene target sequence may be deleted, amplified or otherwise modified within the plant genome. In the preferred embodiment, a deletion of the target sequence flanked by the directly repeated sequence will result.

Alternatively, directly repeated DNA sequence mediated alterations of transgene insertions may be produced in somatic cells. Preferably, recombination occurs in a cultured cell, e.g., callus, and may be selected based on deletion of a negative selectable marker gene, e.g., the periA gene isolated from Burkholderia caryolphilli which encodes a phosphonate ester hydrolase enzyme that catalyzes the hydrolysis of glyceryl glyphosate to the toxic compound glyphosate (U.S. Pat. No. 5,254,801).

### VII. Transgenic Photosynthetic Organisms

In another aspect the invention also contemplates a transgenic organism comprising:

i) a first nucleic acid sequence comprising a first heterologous polynucleotide sequence encoding a carbonic anhydrase enzyme which either a) inherently comprises a first protein-protein interaction domain partner, or b) is fused in frame to a first heterologous protein-protein domain partner;
ii) a second nucleic acid sequence comprising a second heterologous polynucleotide sequence encoding a RubisCO protein subunit operatively coupled to a second protein-protein interaction partner;
wherein the first protein-protein interaction partner and said second protein-protein interaction partner, or the first heterologous protein-protein domain partner and the second protein-protein interaction partner can associate to form a protein complex.

The transgenic organisms therefore contain one or more DNA constructs as defined herein as a part of the plant, the DNA constructs having been introduced by transformation of the photosynthetic organism.

In some embodiments, such transgenic organisms are characterized by having a carbon fixation rate which is at least about 10% higher, at least about 20% higher, at least about 30% higher, at least about 40% higher, at least about 60% higher, at least about 80% higher, or at least about 100% higher than corresponding wild type photosynthetic organisms.

In some embodiments, such transgenic organisms are characterized by having a growth rate which is at least about 10% higher, at least about 20% higher, at least about 30% higher, at least about 40% higher, at least about 60% higher, at least about 80% higher, or at least about 100% higher than corresponding wild type photosynthetic organisms at limiting (less than about 200 ppm carbon dioxide concentrations).

In some embodiments, such transgenic organisms are characterized by having a growth rate which is at least about 10% higher, at least about 20% higher, at least about 30% higher, at least about 40% higher, at least about 60% higher, at least about 80% higher, or at least about 100% higher than corresponding wild type photosynthetic organisms when grown at elevated temperatures. (i.e. in different aspects at elevated temperatures which are higher than about 24° C. average day time temperature, or higher than about 26° C. average day time temperature, or higher than about 28° C. average day time temperature, or higher than about 30 C. average day time temperature, or higher than about 32° C. average day time temperature, or higher than about 34° C. average day time temperature, or higher than about 36° C. average day time temperature).

In some embodiments, such transgenic organisms are characterized by increased carboxylase activity of RubisCO compared to the host control by at least about any of about 10%, about 15%, about 20%, about 25%, about 50%, about 100%, and about 200%.

In some embodiments, such transgenic organisms are characterized by decreased oxygenase activity of RubisCO compared to the host control by at least about any of about 10%, about 15%, about 20%, about 25%, about 50%, about 100%, and about 200%.

In some embodiments, such transgenic organisms are characterized by increased carbon fixation activity of RubisCO compared to the host control by at least about any of: about 10%, about 15%, about 20%, about 25%, about 50%, about 100%, and about 200%.

In some embodiments, such transgenic organisms are characterized by increased steady state levels of ATP compared to the host control steady state ATP levels measured under similar conditions, by at least about any of: about 10%, about 15%, about 20%, about 25%, about 50%, about 100%, and about 200%.

In any of these transgenic organism characteristics, it will be understood that the organism will be grown using standard growth conditions as disclosed in the Examples, and compared to the equivalent wild type organism.

In one embodiment of these transgenic organisms, the transgenic organism is a C3 plant. In one embodiment of any of these transgenic C3 plants, the plant is selected from the group consisting of tobacco; cereals including wheat, rice and barley; beans including mung bean, kidney bean and pea; starch-storing plants including potato, cassava and sweet potato; oil-storing plants including soybean, rape, sunflower and cotton plant; vegetables including tomato, cucumber, eggplant, carrot, hot pepper, Chinese cabbage, radish, water melon, cucumber, melon, crown daisy, spinach, cabbage and strawberry; garden plants including chrysanthemum, rose, carnation and petunia and Arabidopsis, and trees.

In one embodiment of these transgenic organisms, the transgenic organism is a C4 plant. Examples of C4 plants include, for example, corn, sugar cane and sorghum.

Transgenic organisms of interest include both monocots and dicots. Non-limiting examples of monocots include for example, rice, corn, wheat, palm trees, turf grasses, barley, and oats. Non-limiting examples of dicots include for example, soybean, cotton, alfalfa, canola, flax, tomato, sugar beet, sunflower, potato, tobacco, corn, wheat, rice, lettuce, celery, cucumber, carrot, cauliflower, grape, and turf grasses.

In some embodiments, the transgenic organisms of the present invention include for example, row crops and broadcast crops. Non limiting examples of useful such crops are corn, soybeans, cotton, amaranth, vegetables, rice, sorghum, wheat, milo, barley, sunflower, durum, and oats. Non-limiting examples of useful broadcast crops are sunflower, millet, rice, sorghum, wheat, milo, barley, durum, and oats.

In some embodiments, the transgenic organisms of the present invention include corn (Zea mays), canola (Brassica napus, Brassica rapa ssp.), alfalfa (Adedicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaed), cotton (Gossypium hirsutum), sweet potato (Ipomoea batatus), cassava (Manihot esculentd), coffee (Cofea ssp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus carica), guava (Psidium guajava), mango (Mangifera indica), olive (Olea europaeá), papaya (Carica papaya), cashew (Anacardium occidental), macadamia (Macadamia integrifoliá), almond (Primus amygdalus), sugar beets (Beta vulgaris), oats, barley, vegetables, ornamentals, and conifers.

In some embodiments, the transgenic organisms of the present invention include crop plants, for example, cereals and pulses, maize, wheat, potatoes, tapioca, rice, sorghum, millet, cassava, barley, pea, and other root, tuber, or seed crops. Optionally, the plant is a seed crop, for example, oil-seed rape, sugar beet, maize, sunflower, soybean, and sorghum.

In some embodiments, the transgenic organisms of the present invention include Horticultural plants, for example, lettuce, endive, and vegetable basics including cabbage, broccoli, and cauliflower, and carnations, geraniums, petunias, begonias, tobacco, cucurbits, carrot, strawberry, sunflower, tomato, pepper, chrysanthemum, poplar, eucalyptus, and pine.

In some embodiments, the transgenic organisms of the present invention include grain seeds, including for example, corn, wheat, barley, rice, sorghum, and rye.

In some embodiments, the transgenic organisms of the present invention include oil-seed plants, including for example, canola, cotton, soybean, safflower, sunflower, Brassica, maize, alfalfa, palm, and coconut.

In some embodiments, the transgenic organisms of the present invention include leguminous plants, including for example, guar, locust bean, fenugreek, soybean, garden beans, cowpea, mung bean, lima bean, fava bean, lentils, and chickpea.

In some embodiments, the transgenic organisms of the present invention include plants cultivated for aesthetic or olfactory benefits, including for example, flowering plants, trees, grasses, shade plants, and flowering and non-flowering ornamental plants.

In one embodiment of these transgenic organisms, the transgenic organism is an eukaryotic alga. In one aspect, the alga is selected from the group consisting of Nannochloropsis, Chlorella, Dunaliella, Scenedesmus, Selenastrum, Oscillatoria, Phormidium, Spirulina, Amphora, and Ochromonas.

In certain embodiments, the algae used with the methods, transgenic organisms, and DNA constructs of the invention are members of one of the following divisions: Chlorophyta, Cyanophyta (Cyanobacteria), and Heterokontophyta. In certain embodiments, the algae used with the methods of the invention are members of one of the following classes: Chlorophyceae, Bacillariophyceae, Eustigmatophyceae, and Chrysophyceae. In certain embodiments, the algae used with the methods of the invention are members of one of the following genera: Nannochloropsis, Chlorella, Dunaliella, Scenedesmus, Selenastrum, Oscillatoria, Phormidium, Spirulina, Amphora, and Ochromonas. In one aspect algae of the genus Chlorella is preferred.

Non-limiting examples of algae species that can be used with the methods of the present invention include for example, Achnanthes orientalis, Agmenellum spp., Amphiprora hyaline, Amphora coffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlore lla anitrata, Chlorella antarctica, Chlorella aureoviridis, Chlorella Candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Chlamydomonas moewusii Chlamydomonas reinhardtii Chlamydomonas sp. Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena spp., Franceia sp., Fragilaria crotonensis, Fragilaria sp., Gleocapsa sp., Gloeothamnion sp., Haematococcus pluvialis, Hymenomonas sp., lsochrysis aff. galbana, lsochrysis galbana, Lepocinclis, Micractinium, Micractinium, Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phaeodactylum tricomutum, Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

Some algae species of particular interest include, without limitation: Bacillariophyceae strains, Chlorophyceae, Cyanophyceae, Xanthophyceae, Chrysophyceae, Chlorella, Crypthecodinium, Schizocytrium, Nannochloropsis, Ulkenia, Dunaliella, Cyclotella, Navicula, Nitzschia, Cyclotella, Phaeodactylum, and Thaustochytrid.

Some cyanobacterial species of particular interest include, without limitation: Synechocystis, Anacystis, Synechococcus, Agmenelum, Aphanocapsa, Gloecapsa, Nostoc, Anabaena, and Ffremyllia. Optionally, the photosynthetic host is a purple bacterium, a green sulfur bacterium, a green nonsulfur bacterium, or a heliobacterium.

### EXAMPLES

### Materials and Methods

Algal Strains and Cultural Conditions

Chlamydomonas strains CC424 (cw15, arg2, sr-u-2-60 mt⁻) and CC 4147 (FUD7 mt+) were obtained from the Chlamydomonas culture collection at Duke University, USA. Strains were grown mixotrophically in liquid or on solid TAP Medium (Harris, et al., (1989) Genetics 123:281-92) at 23° C. under continuous white light (40 μE m⁻²s⁻¹), unless otherwise stated. Medium was supplemented with 100 μg/mL of arginine when required. Selection of nuclear transformants was performed by using solid TAP medium or TAP medium supplemented with 100 μg/mL of arginine and 50 μg/mL of paromomycin or 25 μg/mL of hygromycin. Selection of chloroplast transformants using strain CC741 (ac-u-(beta) mt+) was performed with high salt (HS) medium.

Nuclear Transformation of C. rienhardtii

Chlamydomonas reinhardtii nuclear transformation was performed using the glass bead method (Kindle, K. L. (1990) Proc Natl Acad Sci USA 87:1228-32). Briefly, CC424 strain of Chlamydomonas was grown in 100 mL of TAP liquid media supplemented with arginine Cells were harvested in log phase (OD₇₅₀=0.8 to 1.0) by centrifugation at 4000 rpm and resuspended in 4 mL of sterile TAP+40 μM sucrose. Resuspended cells (300 μL) were transferred to a sterile micro-centrifuge tube containing 300 mg of sterile glass beads (0.425-0.6 mm, Sigma, USA), 100 μL of sterile 20% PEG 6000 (Sigma, USA) was added to the cells along with 1.5 μg of plasmid DNA. Prior to transformation, all the constructs were restriction digested either to linearize the construct or to excise the two expression cassettes carrying selection marker and gene of interest together, from the plasmid backbone. Following addition of plasmid DNA, cells were vortexed for 20 seconds and plated on to TAP agar plates containing 50 μg/mL paromomycin and 100 μg/mL arginine or 10 μg/mL hygromycin and 100 μg/mL arginine.

For plasmid lacking any selection marker (pSSCR7 backbone), co-transformation was done. For co-transformation, CC424 strain was transformed using glass beads method following addition of the linearized target plasmid (3 μg DNA) and the plasmid harboring the Arg7 gene, p389 (1 μg DNA). Cells were plated on TAP agar plates without arginine.

Chlamydomonas Chloroplast Transformation

Chlamydomonas chloroplast transformation was performed following the protocol described by Ishikura et al., (Ishikura, et al., (1999) J Biosci Bioeng 87:307-14). Briefly, psbA deletion strain (CC741) of Chlamydomonas was grown in 100 mL of TAP liquid media. Cells were harvested in log phase (OD₇₅₀=0.8 to 1.0) by centrifugation at 4000 rpm and resuspended in 2 mL of sterile HS medium. About 300 μL of cells were spread in the center of HS agar plates. Gold particles (1 μm) (InBio Gold, Eltham, Victoria, Australia) coated with plasmid DNAs were shot into Chlamydomonas cells on the agar plate using a Bio-Rad PDS 1000 He Biolistic gun (Bio-Rad, Hercules, Calif., USA) at 1100 psi under vacuum. Following shooting, cells were plated onto HS agar plates for selection.

Genomic DNA was extracted from putative transformants growing on selection medium using a modified xanthine mini prep method described in Newman et al., (1990) Genetics 126(4):875-88. A half loop of algal cells were resuspended in 300 μL of xanthogenate buffer (12.5 mM potassium ethyl xanthogenate, 100 mM Tris-HCl pH 7.5, 80 mM EDTA pH 8.5, 700 mM NaCl) and incubated at 65° C. water for 1.0 hour. Following incubation, the cell suspension was centrifuged for 10 minutes (14,000 rpm) to collect the supernatant. The supernatant was transferred to a fresh micro-centrifuge tube and 2.5 volume of cold 95% ethanol (750 μL) was added. The solution was mixed well by inverting the tube several times allowing DNA to precipitate. The samples were then centrifuged for 5 min (14,000 rpm) to pellet the DNA. The DNA pellet was washed with 700 μL of cold 70% ethanol and centrifuged for 3.0 min. The ethanol was removed by decanting and the DNA pellet was dried using a speedvac to get rid of any residual ethanol. The DNA pellet was then resuspended in 100 μL of sterile double distilled water and 2-5 μL of the DNA sample was used as template for setting PCR.

### Example 1

### Expression of Carbonic Anhydrase (CA) in Algae Increases Biomass

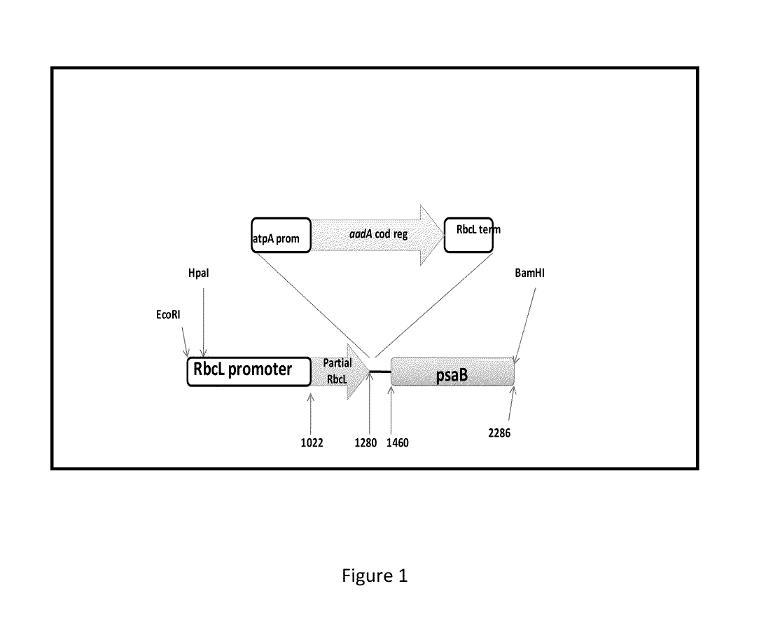

To test the hypothesis that the rate of photosynthetic CO₂ fixation could be increased in algae by expression of a catalytically more active CA in the chloroplast stroma we first constructed a transgenic Chlamydomonas strain in which the endogenous rbcL was partially deleted by transforming the cells with the construct shown in FIG. 1. The resulting strain (DEVL-18) requires transformation with a function rbcL gene for light-dependent growth.

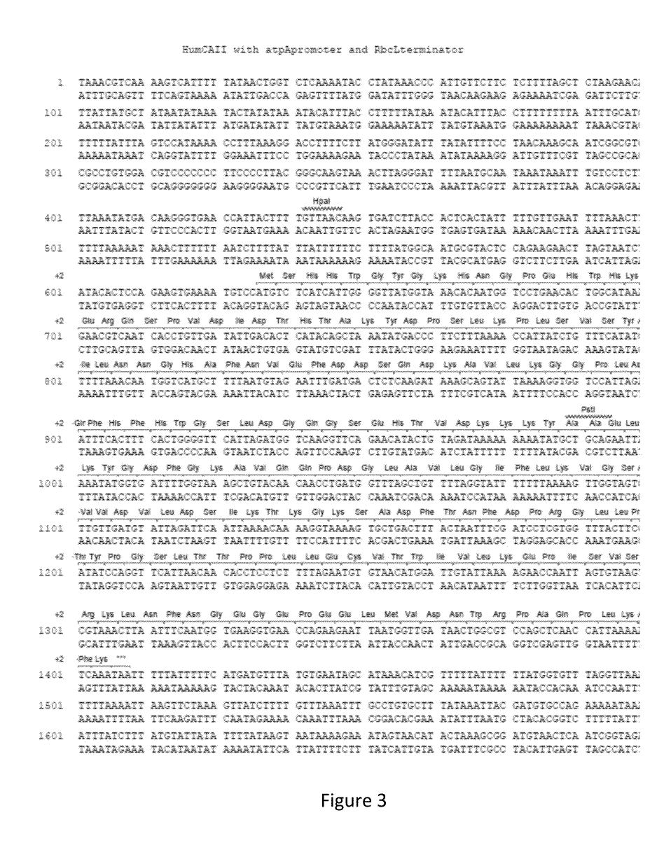

To introduce the human CA-II gene into the chloroplast genome of this strain cells were transformed with an expression vector, in which a codon optimized CA-II gene was operably linked to a chloroplast promoter (atpA) (See FIGS. 2 and 3) to enable stromal expression within the chloroplast. The vector also contained a full length rbcL gene for selection of a transformed host.

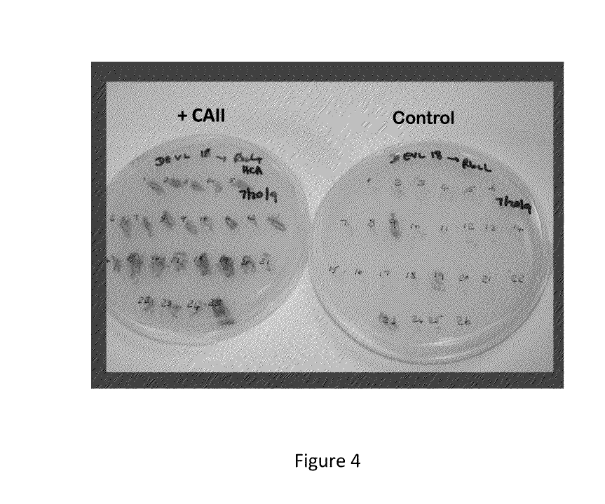

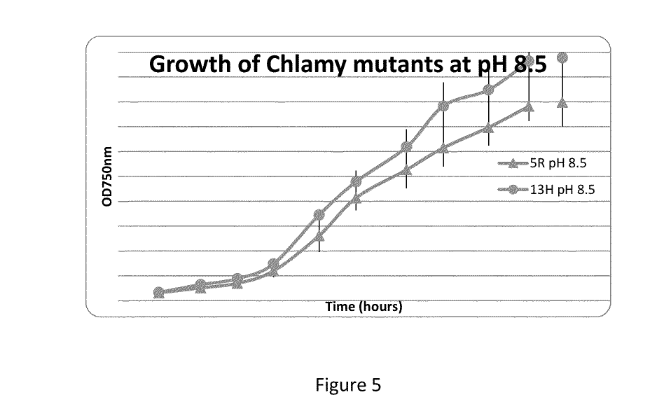

As depicted in FIG. 4 and FIG. 5 the transgenic algae displayed increased growth rates and biomass compared to the control host. FIG. 4 shows the elative colony growth of transgenic Chlamydomonas cells expressing Human CA-II and wild-type cells (—CA).

FIG. 5 demonstrates the expression of an alpha CA to increase growth rates by at least 12% (A750). The graph compares Chlamydomonas cells 5R (LS RubisCO complemented WT strain) and 13H (LS RubisCO complemented WT plus human CAII) in HS media. The graph shows the Relative colony growth of transgenic Chlamydomonas cells expressing Human CA-II and wild-type cells (—CA) when grown at pH 8.5.

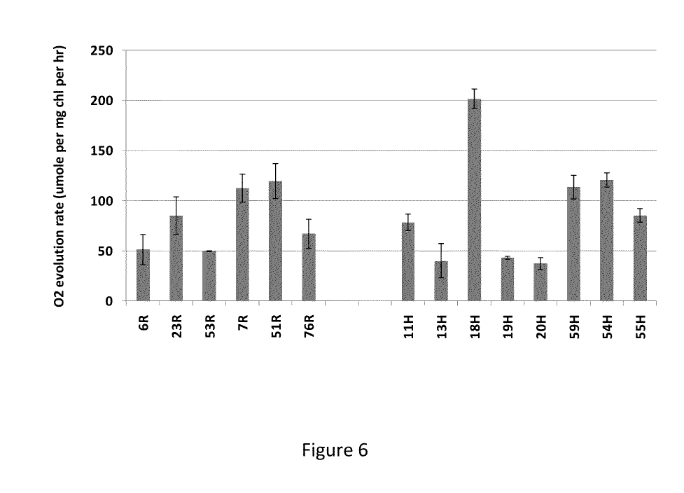

FIG. 6 demonstrates the increase in photosynthesis, as measured by oxygen evolution rate, in transgenic cells expressing the genes encoding the RubisCO large subunit and hCAI compared to transgenic cell expressing only the RubisCO large subunit gene. 6R, 23R, 53R, 7R, 51R, and 76R are complemented with full length RbcL. 11H, 13H, 18H, 19H, 20H, 59H, 54H, and 55H have full length RbcL and hCAII.

Analysis of photosynthetic rates of multiple independent transgenics indicated that those lines expressing human CA-II had on average a 43% higher net photosynthetic rate than wild-type transgenics and a 2× higher photosynthetic rate between the lowest rate for wild-type transgenics and the highest rate for transgenics expressing human CA-II).

Without being bound by theory, it is believed that expression of an alpha CA (CAII), which has a high catalytic efficiency (K_{cat}), increased the chloroplastic CO₂ concentration to levels high enough to inhibit competitively the oxygenase activity of RubisCO, thereby increasing the efficiency of CO₂ fixation and biomass yield.

These results suggested that for those organisms that concentrate inorganic carbon having a more active chloroplastic CA could enhance net photosynthesis.

### Example 2

### RubisCO-Protein-Protein Interaction Fusion Protein

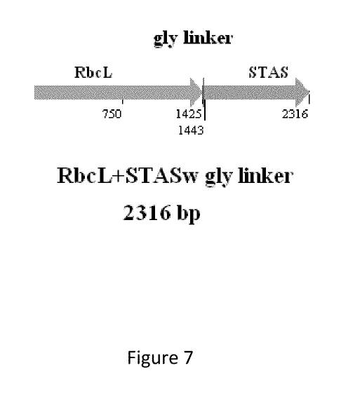

A transforming construct is provided which comprises either a RubisCO SS or LS subunit, for example, from Chlamydomonas reinhardttii or type I RubisCO (for example as disclosed in Tables D7 to D9) fused to a protein-protein interaction (for example, as disclosed in Tables D10 or Table D11. In one embodiment, a STAS domain is fused to the C-terminus of the RubisCO as disclosed in FIG. 3 (SEQ. ID. No. 82). In certain embodiments, the STAS domain is fused to the RubisCO with a linker (e.g. glycine linker), for example, as set forth in SEQ. ID. NO. 84, and FIG. 7). The RubisCO fusion is operably linked to, for example, either an LHCII promoter for nuclear expression or a RubisCO large subunit promoter for chloroplast expression.

### Example 3

### Transformation of a Photosynthetic Host

### The Construct Described in Example 1

is transformed into a host (e.g. DEVL-18 of Example 1) by particle bombardment. The photosynthetic host exhibits enhanced carbon fixation and/or oxygen-evolving activity and biomass yield, particularly at high pHs favoring bicarbonate accumulation in water.

### Example 4

### Alpha type CA

A construct is provided which comprises a mammalian CAII gene. For integration into the chloroplast genome, the gene is operably linked to a chloroplast promoter such as atpA. For integration into the nuclear genome, the gene is operably linked to a promoter such as rbcs and the CA gene is fused to a stromal targeting sequence such as the transit sequence from ssRubisCO.

### Example 5

### Transformation of a Photosynthetic Host

The constructs described in Examples 1 and 3 are selected for transforming a host (e.g. Chlamydomonas DEVL strain or other algal species). The constructs provided in separate transforming vectors or together in a single transforming vector and both genes may be driven by the same or separate promoters and terminators.

For selection in a rbcL partial deletion host strain, an exemplary vector is constructed, as shown in Error! Reference source not found. The host is transformed by particle gun bombardment.

This photosynthetic host exhibits enhanced carbon fixation such as increased biomass compared to a control host.

1-52. (canceled)

53. A genetically modified photosynthetic organism having increased carbon fixation comprising a heterologous polynucleotide sequence which encodes a fusion protein of ribulose-1,5-bisphosphate carboxylase oxygenase (RuBisCO) and a protein-protein interaction domain operably linked to a promoter sequence.

54. The photosynthetic organism of claim 53 wherein said RuBisCO sequence further comprises:
(a) a polynucleotide of SEQ ID NO:82;
(b) a polynucleotide having at least 90% sequence identity across the entire sequence to SEQ ID NO:82;
(c) a polynucleotide amplified from a nucleic acid library using primers which selectively hybridize, under stringent hybridization conditions, to a sequence within a polynucleotide of SEQ ID NO:82; or
(d) a polynucleotide which is a full length complement of a polynucleotide of (a) (b), or (c).

55. The photosynthetic organism of claim 53 wherein said protein-protein interaction domain of said fusion protein is a STAS domain.

56. The photosynthetic organism of claim 53 further comprising a second heterologous polynucleotide sequence which encodes a high activity carbonic anhydrase operably linked to a promoter sequence.

57. The photosynthetic organism of claim 53 wherein said heterologous polynucleotide sequence further comprises a sequence that encodes a high activity carbonic anhydrase operably linked to a promoter sequence.

58. The photosynthetic organism of claim 56 wherein said second recombinant polynucleotide construct further encodes a protein-protein interaction domain that forms a protein-protein interaction pair with the protein-protein interaction domain of the RuBisCO fusion protein.

59. The photosynthetic organism of claim 557 wherein said high activity carbonic anhydrase comprises a human carbonic anhydrase II.

60. The photosynthetic organism of claim 57 wherein said high activity carbonic anhydrase comprises a polynucleotide having at least 90% sequence identity across the entire sequence to SEQ ID NO:1.

61. The photosynthetic organism of claim 53 wherein said RuBisCO is a large subunit RuBisCO.

62. The photosynthetic organism of claim 53 wherein said RuBisCO is a small subunit RuBisCO.

63. The photosynthetic organism of claim 60 further comprising a heterologous polynucleotide sequence that encodes a RuBisCO large subunit and a heterologous polynucleotide sequence that encodes a high activity carbonic anhydrase.

64. The photosynthetic organism of claim 63 wherein the heterologous polynucleotide sequence encoding at least two of said small subunit RuBisCO, said large subunit RuBisCO. and said carbonic anhydrase also encodes a protein-protein interaction domain.

65. The photosynthetic organism of claim 64 wherein the protein-protein interaction domain encoded by the heterologous polynucleotide sequence encoding at least two of said small subunit RuBisCO, said large subunit RuBisCO, and said carbonic anhydrase is a STAS domain.

66. The photosynthetic organism of claim 63 wherein said small subunit RuBisCO, said large subunit RuBisCO, and said carbonic anhydrase are encoded by the same heterologous polynucleotide.

67. The photosynthetic organism of claim 53 wherein said promoter sequence is a chloroplast promoter.

68. A plant part or tissue of the photosynthetic organism of claim 53.

69. A method for increasing carbon fixation in a photosynthetic organism comprising:
introducing into a photosynthetic organism an expression cassette comprising a heterologous polynucleotide sequence which encodes a fusion protein of ribulose-1,5-bisphosphate carboxylase oxygenase (RuBisCO) and a protein-protein interaction domain operably linked to a promoter sequence.

70. The method of claim 69 wherein said RuBisCO sequence further comprises:
(a) a polynucleotide of SEQ ID NO:82;
(b) a polynucleotide having at least 90% sequence identity across the entire sequence to SEQ ID NO:82;
(c) a polynucleotide amplified from a nucleic acid library using primers which selectively hybridize, under stringent hybridization conditions, to a sequence within a polynucleotide of SEQ ID NO:82; or
(d) a polynucleotide which is a full length complement of a polynucleotide of (a), (h), or (c).

71. The method of claim 69 wherein said protein-protein interaction domain of said fusion protein is a STAS domain.

72. The method of claim 69 further comprising introducing a heterologous polynucleotide sequence that encodes a high activity carbonic anhydrase operably linked to a promoter sequence.

73. The method of claim 72 wherein said second recombinant polynucleotide construct that encodes a high activity carbonic anhydrase further encodes protein-protein interaction domain that forms a protein-protein interaction pair with the protein-protein interaction domain of the RuBisCO fusion protein.

74. The method of claim 72 wherein said high activity carbonic anhydrase comprises a human carbonic anhydrase II.

75. The method of claim 72 wherein said high activity carbonic anhydrase comprises a polynucleotide having at least 90% sequence identity across the entire sequence to SEQ NO:1

76. The method of claim 69 wherein said RuBisCO is a large subunit RuBisCO.

77. The method of claim 69 wherein said RuBisCO is a small subunit RuBisCO.

78. The method of claim 77 further comprising introducing a heterologous polynucleotide sequence that encodes a RuBisCO large subunit and a heterologous polynucleotide sequence that encodes a high activity carbonic anhydrase.

79. The method of claim 78 wherein the heterologous polynucleotide sequence encoding at least two of said small subunit RuBisCO, said large subunit RuBisCO, and said carbonic anhydrase also encodes a protein-protein interaction domain.

80. The method of claim 79 wherein the protein-protein interaction domain encoded by the heterologous polynucleotide sequence encoding at least two of said small subunit RuBisCO, said large subunit RuBisCO, and said carbonic anhydrase is a STAS domain.

81. The method of claim 77 wherein said small subunit RuBisCO, said large subunit RuBisCO, and said carbonic anhydrase are encoded by the same expression cassette.

82. The method of claim 69 wherein said promoter sequence is a chloroplast promoter.

83. The method of claim 69, wherein the expression cassette is introduced by a method selected from one of the following: electroporation, micro-projectile bombardment and Agrobacterium-mediated transfer.

84. An isolated polynucleotide comprising a nucleotide sequence encoding a fusion protein of ribulose-1,5-bisphosphate carboxylase oxygenase (RuBisCO) and a protein-protein interaction domain.

85. The isolated polynucleotide of claim 84 wherein said RuBisCO sequence further comprises:
(a) a polynucleotide of SEQ ID NO:82;
(b) a polynucleotide having at least 90% sequence identity across the entire sequence to SEQ ID NO:82;
(c) a polynucleotide amplified from a nucleic acid library using primers which selectively hybridize, under stringent hybridization conditions, to a sequence within a polynucleotide of SEQ ID NO:82; or
(d) a polynucleotide which is a full length complement of a polynucleotide of (a), (b), or (c).

86. The photosynthetic organism of claim 84 wherein said protein-protein interaction domain of said fusion protein is a STAS domain.

87. The photosynthetic organism of claim 84 further comprising a second heterologous polynucleotide sequence which encodes a high activity carbonic anhydrase operably linked to a promoter sequence.

88. The photosynthetic organism of claim 84 wherein said heterologous polynucleotide sequence further comprises a sequence that encodes a high activity carbonic anhydrase operably linked to a promoter sequence.

89. The photosynthetic organism of claim 86 wherein said second recombinant polynucleotide construct further encodes a protein-protein interaction domain that forms a protein-protein interaction pair with the protein-protein interaction domain of the RuBisCO fusion protein.

90. The photosynthetic organism of claim 87 wherein said high activity carbonic anhydrase comprises a human carbonic anhydrase II.

91. The photosynthetic organism of claim 87 wherein said high activity carbonic anhydrase comprises a polynucleotide having at least 90% sequence identity across the entire sequence to SEQ ID NO:1.

92. The photosynthetic organism. of claim 84 wherein said RuBisCO is a large subunit RuBisCO.

93. The photosynthetic organism of claim 84 wherein said RuBisCO is a small subunit RuBisCO.

94. The photosynthetic organism of claim 92 further comprising a heterologous polynucleotide sequence that encodes a RuBisCO large subunit and a heterologous polynucleotide sequence that encodes a high activity carbonic anhydrase.

95. The photosynthetic organism of claim 94 wherein the heterologous polynucleotide sequence encoding at least two of said small subunit RuBisCO, said large subunit RuBisCO, and said carbonic anhydrase also encodes a protein-protein interaction domain.

96. The photosynthetic organism of claim 96 wherein the protein-protein interaction domain encoded by the heterologous polynucleotide sequence encoding at least two of said small subunit RuBisCO, said large subunit RuBisCO, and said carbonic anhydrase is a STAS domain.

97. The photosynthetic organism of claim 95 wherein said small subunit RuBisCO, said large subunit RuBisCO, and said carbonic anhydrase are encoded by the same heterologous polynucleotide.

98. The photosynthetic organism of claim 84 wherein said promoter sequence is a chloroplast promoter.

99. A plant part or tissue of the photosynthetic organism of claim 84.
